(19) United States
(12) Patent Application Publication
(10) Pub. No.: US 2024/0408240 A1
(43) Pub. Date: Dec. 12, 2024

(54) TREATMENT OF HEREDITARY ANGIOEDEMA WITH AAV GENE THERAPY VECTORS AND THERAPEUTIC FORMULATIONS

(71) Applicant: BIOMARIN PHARMACEUTICAL INC., Novato, CA (US)

(72) Inventors: Peter COLOSI, San Anselmo, CA (US); JooChaun ANG, San Francisco, CA (US); Saeed MOSHASHAEE, Novato, CA (US); Stephanie VARGAS, Novato, CA (US); Thomas MACHING, Novato, CA (US); Jack BROWNRIGG, Novato, CA (US); Lawrence Mason SHIH, Novato, CA (US); Iris CHEN, Novato, CA (US); Heather Ann WENZEL, Novato, CA (US)

(21) Appl. No.: 18/697,587

(22) PCT Filed: Sep. 30, 2022
(86) PCT No.: PCT/US2022/077372

§ 371 Date: Apr. 1, 2024

Related U.S. Application Data
(60) Provisional application No. 63/251,558, filed on Oct. 1, 2021.

(51) Int. Cl. A61K 48/00; A61K 9/00; A61K 38/55; A61K 45/06; A61K 47/10; A61K 47/18; A61K 47/26; A61P 7/10; C12N 15/86

(52) U.S. Cl. CPC A61K 48/0058; A61K 9/0019; A61K 38/55; A61K 45/06; A61K 47/10; A61K 47/18; A61K 47/26; A61K 48/0075; A61K 48/0083; A61P 7/10; C12N 15/86; C12N 2750/14143; C12N 2750/14145; C12N 2830/008; C12N 2830/50

(57) ABSTRACT

Provided herein are pharmaceutical compositions and methods for treating hereditary angioedema in a human subject.

### FIELD

The invention relates to methods of treating hereditary angioedema (HAE) by administering gene therapy vectors expressing functional human C1 esterase inhibitor (hC1-INH) to alleviate the deficiency in circulating levels of functional C1-INH in subjects having hereditary angioedema, as well as therapeutic formulations comprising the same.

### BACKGROUND

Hereditary angioedema (HAE) is a genetic disorder characterized by acute, recurring and self-limiting edematous crises episodes that may affect multiple locations of the body. The crises present with symptoms of urticaria and/or angioedema, including but not limited to swelling of the skin and/or mucous membranes (subcutaneous edema or submucosal edema), including the respiratory and gastrointestinal tracts. Swelling of the larynx can cause fatal asphyxiation. Recurrent episodes of severe swelling can affect face, extremities, intestinal tract and airway which are painful, disfiguring, and sometimes life threatening if they obstruct respiration. HAE is caused by mutations in the SERPING1 gene which encodes for complement 1 Esterase Inhibitor (C1-INH) protein leading to decreased C1-INH levels (Type I HAE) or normal or elevated levels of mutated dysfunctional C1-INH (Type II HAE), called HAE with C1-inhibitor deficiency. There is a third type of HAE (formerly called Type III HAE) in which patients are found to have normal C1-INH protein but have a mutation in other genes (i.e., the Factor XII gene) which causes the HAE (also called HAE with normal C1-Inhibitor). Alternatively, this can also be called HAE with normal C1-inhibitor. C1-INH is a broad-spectrum serine protease inhibitor that regulates the complement, contact (kallikrein/kinin system), coagulation and fibrinolytic pathways through the inhibition of multiple proteases involved in these pathways. Lumry, Am J Manag Care. 19(7 SUPPL), S103-110 (2013), Zuraw, N Engl J Med. 359:1027 36 (2008). It is a major inhibitor of several complement proteases such as C1r and C1s and contact proteases, including factor XIIa and kallikrein, and a minor inhibitor of fibrinolytic proteases such as plasmin and factor XIa. Deficiency in functional plasma C1-INH leads to unregulated activation of the complement pathway and/or contact activation pathway. In patients with HAE, uninhibited activation of the contact pathway due to insufficient levels of functional C1-INH results in unregulated cleavage of high molecular weight kininogen by kallikrein, leading to generation of excessive free bradykinin, a potent vasoactive peptide that increases capillary permeability and edema. See, e.g., Riedl M. Clin Drug Investig. 35(7): 407-17 (2005)). If left untreated, the condition has a 25% mortality rate. HAE is estimated to affect 1 in 50,000-100,000 individuals globally.

HAE attacks can be triggered by minor surgical or dental procedures or trauma, infection, stress, and the use of medications, especially inhibitors of angiotensin-converting enzyme (ACE) and estrogens. Acute attacks are typically treated with either plasma-derived or recombinant C1-INH protein, fresh frozen plasma, ecallantide (a kallikrein inhibitor) and/or icatibant (a bradykinin B2 receptor antagonist). Conventional prophylactic therapy includes plasma-derived C1-INH protein intravenously or subcutaneously, attenuated androgens such as danazol, antifibrinolytic agents, progesterone, a humanized, monoclonal antibody against plasma kallikrein (lanadelumab), or an oral kallikrein inhibitor (berotralstat) although each of these has adverse effects. Treatment of pregnant women represents a problem because androgens, antifibrinolytic agents and other HAE medications are contraindicated during pregnancy, delivery and lactation and only plasma-derived C1-inhibitor can be given safely in these instances.

Despite recent advances in the management of HAE, including prophylactic treatment options, patients may still experience severe breakthrough attacks that can cause disabling pain and significant morbidity. Within the current treatment landscape, there remains a substantial unmet medical need for therapies that have the potential to correct the phenotype of HAE by addressing its underlying cause.

### SUMMARY

The present disclosure provides methods of treating or preventing hereditary angioedema, as well as methods of increasing or normalizing levels of functional C1-INH, by administering recombinant AAV (rAAV) particles to a subject having hereditary angioedema in an amount effective to provide a preventive effect against occurrence of HAE attacks. For example, different SERPING-1 mutations having a dominant negative effect can be overcome by using the methods and treatments provided in the present disclosure. In addition, the method is suitable to correct the dominant negative effect of certain mutations of the SERPING-1 gene in type I and II HAE patients and thus can restore the levels of functional C1-INH from the unaffected SERPING-1 gene or functional C1-inhibitor to normal or close to normal levels.

In one aspect, the disclosure provides a method of increasing plasma functional C1-INH levels in a human subject in need thereof, comprising administering to the subject a therapeutically effective amount of a recombinant adeno-associated virus (rAAV) particle comprising an AAV capsid, preferably an AAV capsid with liver tropism, and a recombinant AAV vector construct comprising a nucleic acid encoding a functional human C1-INH operatively linked to a heterologous liver-selective or liver-specific transcription regulatory region.

The disclosure also provides a method of treating a human subject with hereditary angioedema (HAE), comprising administering to the subject a single dose ranging from about 2E13 vector genomes/kilogram body weight of the subject (vg/kg) to about 6E14 vg/kg of a recombinant adeno-associated virus (rAAV) particle comprising (a) an AAV capsid with liver tropism, and (b) a recombinant vector construct comprising a nucleic acid encoding a functional C1 esterase inhibitor (C1-INH) protein operatively linked to a heterologous liver-specific transcription regulatory region.

In any of the methods disclosed herein, the dose of rAAV particle may be about 2E13 vg/kg; about 6E13 vg/kg; about 2E14 vg/kg; about 4E14 vg/kg or about 6E14 vg/kg.

International Patent Publication No. WO-2021/097157 (PCT/US2020/060337), incorporated herein by reference in its entirety, discloses C1-INH encoding nucleic acid sequences, liver-specific transcription regulatory regions, enhancers, promoters, introns, polyadenylation signals, and other vector elements, AAV recombinant vector constructs and virus particles.

In the recombinant vector construct comprising a nucleic acid encoding a functional C1 esterase inhibitor (C1-INH) protein, the functional C1-INH protein may comprise an amino acid sequence at least 95%, 98% or 99% identical to amino acids 23 through 500 of SEQ ID NO: 2. In some embodiments, the nucleic acid encoding the functional C1-INH comprises a nucleotide sequence at least 80%, 85%, 90%, 95%, 98% or 99% identical to SEQ ID NO: 1. The liver-specific transcription regulatory region may comprise a fragment of an hAAT promoter and/or a fragment of an HCR enhancer/ApoE enhancer. In some embodiments, the liver-selective or liver-specific transcription regulatory region comprises a nucleotide sequence at least 90%, 95%, 98% or 99% identical to SEQ ID NO: 3 or SEQ ID NO: 15. The liver-specific transcription regulatory region may further comprise a nucleotide sequence at least 90%, 95%, 98% or 99% identical to SEQ ID NO: 4. In some embodiments, the liver-specific transcription regulatory region comprises a nucleotide sequence at least 90%, 95%, 98% or 99% identical to SEQ ID NO: 5. The recombinant vector construct may comprise an intron, e.g. one or more native C1-INH introns or fragment thereof, or optionally a beta globin intron or fragment thereof, or an hAAT intron or fragment thereof, or a combination thereof, preferably within the C1-INH coding sequence. In some embodiments, the intron comprises a nucleotide sequence at least 90%, 95%, 98% or 99% identical to SEQ ID NO: 64. In some embodiments, the intron comprises a nucleotide sequence at least 90%, 95%, 98% or 99% identical to SEQ ID NO: 6. The recombinant vector construct may further comprise a polyadenylation signal, for example, a bovine growth hormone (bGH) (SEQ ID NO: 19) or human growth hormone (hGH) (SEQ ID NO: 7) polyadenylation signal.

In the methods disclosed herein, the subject may be administered a population of rAAV particles produced by a method comprising (a) providing an insect cell comprising one or more nucleic acid constructs comprising: (i) a recombinant vector construct comprising (1) a 5′ AAV ITR and a 3′ AAV ITR, (2) a heterologous liver-specific transcription regulatory region comprising a nucleotide sequence at least 90% identical to SEQ ID NO: 4 and a nucleotide sequence at least 90% identical to SEQ ID NO: 3, (3) a nucleic acid encoding a functional C1-INH comprising an amino acid sequence at least 95% identical to amino acids 23 through 500 of SEQ ID NO: 2, and (4) a polyadenylation signal; (ii) a nucleotide sequence encoding one or more AAV Rep proteins which is operably linked to a promoter that is capable of driving expression of the Rep protein(s) in the cell; and (iii) a nucleotide sequence encoding one or more AAV5 type capsid protein which is operably linked to a promoter that is capable of driving expression of the capsid protein(s) in the cell; (b) culturing the cell under conditions permitting expression of the Rep and the capsid proteins and production of an AAV particle; and (c) recovering the AAV particle. Optionally, the population is enriched for AAV particles comprising full length or nearly full length vector genomes by steps that reduce the number of empty capsids.

In some embodiments, the recombinant vector construct comprises a nucleotide sequence at least 90%, 95%, 98% or 99% identical to any one of SEQ NOs: 9, 20-36, 57 or 58. In some embodiments, the AAV capsid comprises an amino acid sequence at least 85%, 90% or 95% identical to any one of SEQ ID NOs: 35-51. Preferably, the AAV capsid with liver tropism is an AAV5 type capsid, optionally at least 85%, 90% or 95% identical to SEQ ID NO: 44. Preferably, the AAV capsid comprises an amino acid sequence at least 95%, 97%, 98% or 99% identical to SEQ ID NO: 44.

In any of the methods described herein the rAAV particle may be administered by intravenous infusion.

In any of the methods herein, the subject may have Type I or Type II hereditary angioedema. In some embodiments, prior to rAAV particle administration, the subject has (a) a functional C1-INH level about 50% of the lower limit of normal (LLN) or lower, prior to rAAV particle administration, and/or (b) a C4 complement level below normal range.

In some embodiments, the subject is 18 or more years old, or is a juvenile subject, or is 12 to 18 years old or is male or a nonpregnant female.

As described in Craig, Timothy, et al. “WAO guideline for the management of hereditary angioedema.” World Allergy Organization Journal 5.12 (2012): 182-199, one unit of pdC1-INH is equivalent to the C1-INH content of one milliliter of human plasma (270 milligraris (mg)/Liter (L). So, 100% functional is equal to 1 IU, which is equal to 270 microgram (μg)/milliliter (mL). To this extent, the normal range of functional C1-INH(f) is 70-130% or 160-320 μg/mL. In some embodiments, prior to rAAV particle administration, the subject is a patient having an abnormal C1-INH(f) value that is outside the normal range of functional C1-INH(f). For example, the abnormal C1-INH(f) value is outside the range of 70-130%. In another example, the abnormal C1-INH(f) value is outside the range of 160-320 μg/mL.

In some embodiments, prior to rAAV particle administration, the subject may have suffered HAE attacks at a frequency of at least 1 attack per month on average for at least 6 months and/or (b) may have received long-term prophylactic C1-INH replacement therapy, lanadelumab, berotralstat or any other prophylactic HAE medication for at least 6 months prior to rAAV particle administration.

In some embodiments, prior to rAAV particle administration, the subject is a patient that is at least 1 years old. For example, the patient is an adult or a pediatric patient.

In some embodiments, prior to rAAV particle administration, the subject suffered from low burden or mild HAE, e.g., patients experiencing about 2 or less HAE attacks per year. In some embodiments, prior to rAAV particle administration, the subject suffered from moderate HAE, e.g., patients experiencing about 3 to about 12 HAE attacks per year. In some embodiments, prior to rAAV particle administration, the subject suffered from severe HAE, e.g. patients experiencing 13 or more HAE attacks per year.

In some embodiments, the subject does not have detectable anti-AAV5 capsid antibody in blood prior to rAAV particle administration, e.g., is not AAV5 seropositive. In some embodiments, the subject does not have clinically significant liver disease prior to rAAV particle administration. In some embodiments, the subject has ALT and/or AST levels within normal range prior to rAAV particle administration. In some embodiments, the subject has not received steroids at least 30 days prior to rAAV particle administration.

In any of the methods described herein, preferably the dose is effective to increase the plasma level of C1-INH (preferably functional C1-INH) in the subject by an absolute amount of at least about 10% of functional C1-INH activity (normal range: 70-130% or 0.6-1.3 IU/ml according to WHO Standard for C1-inhibitor). This corresponds to an increase in the plasma level of C1-INH protein in the subject by at least about 20 μg/mL (normal range 160-320 μg/mL). In some embodiments, the dose is effective to increase the plasma level of C1-INH in the subject by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130% or more than lower limit of normal. In some embodiments, the dose is effective to increase the plasma level of C1-INH in the subject by at least about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, or 160 μg/mL. In some embodiments, the dose is effective to increase the plasma level of C1-INH (preferably functional C1-INH) to a level of at least about 160 μg/mL, 170 μg/mL, 180 μg/mL, 190 μg/mL, 200 μg/mL, 210 μg/mL, 220 μg/mL, 230 μg/mL, 240 μg/mL, 260 μg/mL, 270 μg/mL, 280 μg/mL, 290 μg/mL, 300 μg/mL, 310 μg/mL, or 320 μg/mL. In some embodiments, the dose is effective to increase the plasma level of C1-INH to a range between about 160 and about 320 μg/mL. In some embodiments, the dose is effective to increase the plasma level of C1-INH in the subject to approximately 70% of normal function (e.g., 0.7 IU/mL) up to about 130% of normal function. Preferably, the plasma level of C1-INH is less than 150% of normal C1-INH. Preferably, the treatment does not result in significant increased thrombotic risk. In some embodiments, the level of C1-INH is measured by a functional assay. In other embodiments, the level of C1-INH is measured by an antigenic assay. Preferably the dose maintains an increased plasma level for a period of at least about six months. In some embodiments, an increased plasma level is maintained for at least about one year, or 2, 3, 4 or 5 years.

In some embodiments, the dose may be effective to normalize and replace the inhibitory function of C1-INH in the coagulation system on factors XIa, XIIa as well as on plasmin and tissue plasminogen activator of the fibrinolytic system. In some embodiments, the level of coagulation markers found elevated in HAE type I and II patients can be normalized or reduced to levels close to the normal range. In some embodiments, the dose may be effective to reduce the number or severity of acute HAE attacks of the subject, preferably over a period of at least about six months. In some embodiments, the dose is effective to reduce the number of moderate and severe acute HAE attacks of the subject, preferably over a period of at least about six months. In some embodiments, the dose is effective to reduce the number of high morbidity acute HAE attacks of the subject, preferably over a period of at least about six months. In some embodiments, the reduction in HAE attacks is maintained for at least about one year, or 2, 3, 4 or 5 years. In some embodiments, the dose may be effective to reduce the number of or severity of acute HAE attacks of the subject by at least about 5%, 10%, 15%, 20%, 25%, 30%, 35%, 40%, 45%, 50%, 55%, 60%, 65%, 70%, 75%, 80%, 85%, 90%, 95% or 100% over a period of about six months, or at least one year. In some embodiments, the dose may be effective to render patients attack-free, e.g. at least 10%, 15%, 20%, 25%, 30%, 35%, 40%, 45%, 50%, 55%, 60%, 65%, 70%, 75%, 80%, 85%, 90%, or 95% of treated patients are attack-free at 4 months, 6 months or 1 year.

In some embodiments, the dose is effective to reduce the dose of or frequency of administration of HAE-specific therapy to the subject for acute HAE attacks, preferably over a period of at least about six months. In some embodiments, the dose may be effective to reduce the dose or frequency of HAE-specific therapy for acute HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of about six months, or at least one year. HAE-specific therapy includes plasma derived or recombinant C1 INH, bradykinin antagonists such as bradykinin B2 receptor antagonist, kallikrein inhibitors, anti-kallikrein antibodies.

In some embodiments, the dose is effective to reduce the dose of or frequency of administration of HAE-specific prophylactic therapy to the subject, preferably over a period of at least about six months. In some embodiments, the dose may be effective to eliminate prophylactic therapy in at least 10%, 15%, 20%, 25%, 30%, 35%, 40%, 45%, 50%, 55%, 60%, 65%, 70%, 75%, 80%, 85%, 90%, or 95% of treated patients over a period of about six months, or at least one year. In some embodiments, the dose may be effective to reduce the dose of prophylactic therapy by at least about 5%, 10%, 15%, 20%, 25%, 30%, 35%, 40%, 45%, 50%, 55%, 60%, 65%, 70%, 75%, 80%, 85%, 90%, 95% or 100% over a period of about six months, or at least one year. HAE-specific prophylactic therapy includes plasma-derived C1-INH (e.g., CINRYZE, HAEGARDA); recombinant C1-INH; plasma kallikrein inhibitor such as ORLADEYO (berotralstat), anti-kallikrein antibody, such as TAKHZYRO (lanadelumab), or androgens such as danazol, oxandrolone, and stanozolol. In some embodiments, the reduction is maintained for at least about one year, or 2, 3, 4 or 5 years.

In some embodiments, the dose is effective to improve health-related quality of life, optionally as measured by any one or more of Angioedema Quality of Life Questionnaire (AE-QOL) score, Angioedema Control Test (AECT) score, Treatment Satisfaction Questionnaire for Medication (TSQM-9), EuroQoL-5D-5L (EQ-5D-5L) score, or Patient Global Impression of Severity (PSI-S) score, preferably over a period of at least about six months. In some embodiments, the dose may be effective to improve one or more of these measures of the health-related quality of life by at least about 5%, 10%, 15%, 20%, 25%, 30%, 35%, 40%, 45%, 50%, 55%, 60%, 65%, 70%, 75%, 80%, 85%, 90%, 95% or 100% over a period of about six months, or at least one year. In some embodiments, the improvement is maintained for at least about one year, or 2, 3, 4 or 5 years.

In related aspects, the disclosure provides compositions of the recombinant AAV vector construct or AAV particle as described herein for use according to any of the methods disclosed herein. The disclosure also provides uses of a recombinant AAV vector construct or AAV particle as described herein for the preparation of a medicament for treatment according to any of the methods described herein.

The methods of the disclosure may further comprise administering to the subject a prophylactically effective amount of a glucocorticoids or other systemic immunosuppressant to prevent hepatotoxicity (a prophylactic immunosuppressant), prior to detection of hepatotoxicity. In some embodiments, the prophylactically effective amount of glucocorticoid or immunosuppressant is administered concurrent with administration of the rAAV particles of the invention. In other embodiments, the administration of the prophylactically effective amount of glucocorticoid or immunosuppressant begins after administration of the rAAV particles, e.g. starting 3 to 10 weeks after administration of the rAAV particles, but prior to detection of hepatotoxicity.

In some embodiments, the prophylactic immunosuppressant is a glucocorticoid, optionally dexamethasone, prednisone, prednisolone, methylprednisolone, fludrocortisone, hydrocortisone, or budesonide. In some embodiments, the prophylactically effective amount is a prednisone-equivalent dose of from 10 mg/day to 40 mg/day, optionally for a time period of at least about 13 weeks, followed by tapering amounts of the glucocorticoid for a time period of about 3 weeks. The methods may further comprise (a) determining a baseline level of a marker of hepatotoxicity in the blood of the subject prior to rAAV particle administration, optionally about one month prior to said administration, and (b) subsequently determining a post-administration level of said marker for hepatotoxicity in the blood of the subject, optionally every week for at least 12 weeks, or more frequently.

The methods of the disclosure may comprise administering to the subject a therapeutically effective amount of a glucocorticoid or other systemic immunosuppressant to treat hepatotoxicity (a therapeutic immunosuppressant), upon detection of hepatotoxicity. The methods may comprise: (c) upon detection of hepatotoxicity by biochemical or clinical signs, administering to the subject a therapeutically effective amount of a systemic immunosuppressant to reduce hepatotoxicity. The marker(s) of hepatotoxicity may comprise ALT and/or AST. In some embodiments, detection of hepatotoxicity is by (i) a post-administration level of said marker of hepatotoxicity greater than the upper limit of normal (ULN), or (ii) a post-administration level of said marker of hepatotoxicity greater than or equal to twice the baseline level of said marker of hepatotoxicity. In some embodiments, the therapeutic immunosuppressant is a glucocorticoid, optionally dexamethasone, prednisone, prednisolone, fludrocortisone, hydrocortisone, or budesonide. In some embodiments, the therapeutically effective amount is a prednisone-equivalent dose of from 10 mg/day to 40 mg/day, optionally for a time period of at least about 5 weeks, followed by tapering amounts of the glucocorticoid for a time period of about 3 weeks.

In any of the methods herein, plasma functional C1-INH level of the subject may be measured every week, preferably for at least 12 weeks.

In another aspect, the disclosure provides a pharmaceutical composition comprising rAAV particles at a concentration of at least about 1E13 vg/ml to about 1E14 vg/ml, a tris(hydroxymethyl)aminomethane (Tris) buffering agent, an isotonicity agent, a cryopreservative agent and a surfactant which is stable during storage at about −60° C. (minus sixty degrees centigrade) or less for at least about 1 year, 1.5 years, or 2 years. In some embodiments, the pharmaceutical composition comprises rAAV particles at a concentration of at least about 1E13 vg/ml to about 1E14 vg/ml, Tris buffer, trehalose and poloxamer 188 which is stable during storage at about −60° C. (minus sixty degrees centigrade) or less for at least about 1 year, 1.5 years, or 2 years. In some embodiments, the pharmaceutical composition comprises rAAV particles at a concentration of at least about 1E13 vg/ml to about 1E14 vg/ml, Tris buffer at a concentration of about 10 to about 50 mM, sodium chloride at a concentration of about 100 mM to about 165 mM, trehalose at a concentration of about 2 to about 3 wt %, and a poloxamer or polysorbate at a concentration of about 0.05% to about 0.15% w/v. In some embodiments, the pharmaceutical composition comprises rAAV particles at a concentration of at least about 1E13 vg/ml to about 1E14 vg/ml, Tris buffer at a concentration of about 10 to about 30 mM, sodium chloride at a concentration of about 100 mM to about 165 mM, trehalose at a concentration of about 2 to about 3 wt %, and a poloxamer or polysorbate at a concentration of about 0.05% to about 0.15% w/v. Optionally the poloxamer is poloxamer 188. In some embodiments, the Tris buffer is at a concentration of about 15 to about 25 mM, sodium chloride is at a concentration of about 100 to about 140 mM, trehalose is at a concentration of about 2.3 to about 2.7 wt %, and the poloxamer is poloxamer 188 at a concentration of about 0.05% to about 0.15% w/v. Optionally the poloxamer 188 is at a concentration of about 0.1% w/v. In some embodiments, the rAAV particle is at a concentration of about 6E13 vg/ml. In some embodiments, the pharmaceutical composition comprises rAAV particles at a concentration of about 6E13 vg/ml, about 20 mM Tris buffer, about 120 mM sodium chloride, about 2.5 wt % trehalose dihydrate, and about 0.1% w/v poloxamer 188. Preferably, in such pharmaceutical compositions, the rAAV particles comprise an AAV5 type capsid, and a recombinant AAV vector construct as described herein.

Preferably the pharmaceutical composition is a liquid aqueous solution and is for storage at freezing temperature. In any of these embodiments, the composition is for use in intravenous administration of rAAV particle to a patient with hereditary angioedema. In a related aspect, the disclosure provides a method of using the pharmaceutical compositions described herein to treat a subject with hereditary angioedema by administering said pharmaceutical composition by intravenous infusion.

In certain related embodiments, the disclosure provides a composition of a recombinant vector construct or AAV particle as described herein for use for co-administration with the prophylactic administration of immunosuppressant (e.g., glucocorticoids) and/or the therapeutic administration of immunosuppressant (e.g., glucocorticoids) described herein. The disclosure also provides for use of a recombinant vector construct or AAV particle as described herein in preparation of a medicament for co-administration with the prophylactic administration of immunosuppressant and/or the therapeutic administration of immunosuppressant described herein. Similarly, the disclosure provides a composition of an immunosuppressant for the prevention and/or treatment of any hepatotoxicity associated with administration of the AAV particle according to the prophylactic administration of immunosuppressant and/or the therapeutic administration of immunosuppressant described herein. The disclosure also provides for use of an immunosuppressant in preparation of a medicament for the prevention and/or treatment of any hepatotoxicity associated with administration of the AAV particle according to the prophylactic administration of immunosuppressant and/or the therapeutic administration of immunosuppressant described herein.

Other embodiments will be evident to one skilled in the art upon reading the present specification.

### BRIEF DESCRIPTION OF DRAWINGS

FIG. 1 depicts a C1-INH vector construct.

FIG. 2 is a graph showing a thermal based capsid integrity (TBCI) analysis of capsid stability of rAAV C1-INH vector in formulations with different buffers (Tris, Phosphate, and Citrate) and pH (6-9).

FIGS. 3A and 3B are graphs showing percentage of VP1 in the rAAV C1-INH vector in formulations with different buffers (Tris, Phosphate, and Citrate) at pH 7 and 8 after storage for 0 days, 3 days, 1 week, 2 weeks, 1 month, and 2 months.

FIG. 4 is a graph showing the percentage of rAAV C1-INH vector multimers in formulations with different buffers (Tris, Phosphate, and Citrate) at pH 7.4.

FIG. 5 is a graph showing potency (percentage relative to a reference sample) of different rAAV C1-INH vector concentrations (6×10e13 vector genomes (vg)/milliliter (mL) and 2×10e14 vg/mL) in formulations with different buffer concentrations (10 mM Tris, 20 mM Tris, 10 mM Phosphate, and 20 mM Phosphate) after storage for 0 days and 7 days.

FIGS. 6A and 6B are graphs showing a TBCI analysis of capsid stability of different rAAV C1-INH vector concentrations (6×10e13 vg/mL and 2×10e14 vg/mL) that have been purified with or without zonal ultracentrifugation (ZUC) in formulations with different buffer concentrations (10 mM Tris, 20 mM Tris, 10 mM Phosphate, and 20 mM Phosphate) after storage at about 25° C. for 0 days to 30 days.

FIG. 7 is a graph showing the potency of rAAV C1-INH vector in a formulation at pH 7.4 comprising 20 mM Tris buffer, 120 mM sodium chloride, 2.5% trehalose and 0.1% P-188 when stored at −20° C., −40° C., and −70° C. for 0 months to 24 months.

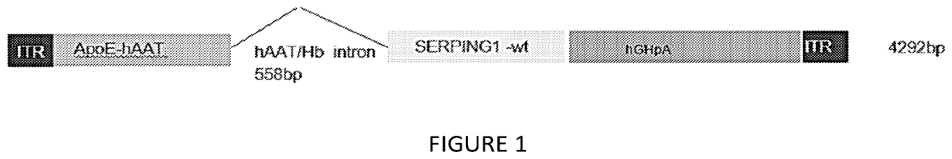

### DETAILED DESCRIPTION

Provided herein are methods of treating human subjects with hereditary angioedema, or with a deficiency in level of functional C1-INH. The methods involve administering a dose of rAAV particles effective to increase or normalize levels of functional C1-INH. Also provided herein are pharmaceutical formulations for use in methods of treating subjects with hereditary angioedema.

Based on clinical studies in HAE patients with IV plasma derived C1 INH concentrate for prevention of HAE attacks, small C1 INH increases of trough levels (approximately 20 μg/mL) were shown to be therapeutic with a clinically meaningful reduction in attacks of greater than 50%. Lumry et al., J. Allergy Clin. Immunol. Pract., 7(5):1610 18 (2019); Berman, Allergy, 70(10):1319 28 (2015).

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

As used herein, in the context of gene delivery, the term “vector” or “gene delivery vector” may refer to a particle that functions as a gene delivery vehicle, and which comprises nucleic acid (i.e., the vector genome comprising any of the vector constructs described herein) packaged within, for example, an envelope or capsid. A gene delivery vector may be a viral gene delivery vector or a non-viral gene delivery vector. Alternatively, in some contexts, the term “vector” may be used to refer only to the vector genome or vector construct. Viral vectors suitable for use herein may be a parvovirus, an adenovirus, a retrovirus, a lentivirus or a herpes simplex virus. The parvovirus may be an adenovirus-associated virus (AAV).

As used herein, the term “AAV” is a standard abbreviation for adeno-associated virus. Adeno-associated virus is a single-stranded DNA parvovirus that grows only in cells in which certain functions are provided by a co-infecting helper virus. There are numerous serotypes of AAV that have been characterized. General information and reviews of AAV can be found in, for example, Carter, 1989, Handbook of Parvoviruses, Vol. 1, pp. 169-228; and Berns, 1990, Virology, pp. 1743-1764, Raven Press, (New York); Gao et al., 2011, Methods Mol. Biol. 807: 93-118; Ojala et al., 2018, Mol. Ther. 26(1): 304-19. However, it is fully expected that these same principles will be applicable to additional AAV serotypes since it is well known that the various serotypes are quite closely related, both structurally and functionally, even at the genetic level. (See, e.g., Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs).

An “AAV viral particle” as used herein refers to an infectious viral particle composed of at least one AAV capsid protein and an encapsidated AAV genome. “Recombinant AAV” or “rAAV”, “rAAV virion” or “rAAV viral particle” or “rAAV vector particle” or “AAV virus” refers to a viral particle composed of at least one capsid or Cap protein and an encapsidated rAAV vector genome (vg) as described herein. In different embodiments, the vg includes a nucleotide encoding a functional therapeutic protein-encoding sequence, e.g. hC1-INH-encoding sequence. If the particle comprises a heterologous polynucleotide encoding hC1-INH to be delivered to a mammalian cell, it can be referred to as an “rAAV vector particle”, an “rAAV vector”, an “AAV C1-INH vector”, or an “rAAV C1-INH vector”.

As used herein, an “AAV vector construct” refers to nucleic acids, either single-stranded or double-stranded, having an AAV 5′ inverted terminal repeat (ITR) sequence and an AAV 3′ ITR flanking a protein-coding sequence (in different embodiments, a functional therapeutic protein-encoding sequence, e.g. hC1-INH-encoding sequence) operably linked to transcription regulatory elements (also called “expression control elements”) that are heterologous to protein-encoding sequence and/or heterologous to the AAV viral genome, i.e., one or more promoters and/or enhancers and, optionally, a polyadenylation sequence and/or one or more introns inserted between exons of the protein-coding sequence. A single-stranded AAV vector refers to nucleic acids that are present in the genome of an AAV virus particle, and can be either the sense strand or the anti-sense strand of the nucleic acid sequences disclosed herein. The size of such single-stranded nucleic acids is provided in bases. A double-stranded AAV vector refers to nucleic acids that are present in the DNA of plasmids, e.g., pUC19, or genome of a double-stranded virus, e.g., baculovirus, used to express or transfer the AAV vector nucleic acids. The size of such double-stranded nucleic acids in provided in base pairs (bp).

While AAV particles have been reported in the literature having AAV genomes of >5.0 kb, in many of these cases the 5′ or 3′ ends of the encoded genes appear to be truncated (see Hirsch et al., Molec. Ther. 18:6-8, 2010, and Ghosh et al., Biotech. Genet. Engin. Rev. 24:165-178, 2007). It has been shown, however, that overlapping homologous recombination occurs in AAV infected cells between nucleic acids having 5′ end truncations and 3′ end truncations so that a “complete” nucleic acid encoding the large protein is generated, thereby reconstructing a functional, full-length gene.

Oversized AAV vectors are randomly truncated at the 5′ ends and lack a 5′ AAV ITR. Because AAV is a single-stranded DNA virus, and packages either the sense or antisense strand, the sense strand in oversized AAV vectors lacks the 5′ AAV ITR and possibly portions of the 5′ end of the target protein-coding gene, and the antisense strand in oversized AAV vectors lacks the 3′ ITR and possibly portions of the 3′ end of the target protein-coding gene. A functional transgene is produced in oversized AAV vector infected cells by annealing of the sense and antisense truncated genomes within the target cell. Thus, in certain embodiments, the AAV C1-INH vectors and/or viral particles comprise at least one ITR.

The term “inverted terminal repeat (ITR)” as used herein refers to the art-recognized regions found at the 5′ and 3′ termini of the AAV genome which function in cis as origins of DNA replication and as packaging signals for the viral genome. AAV ITRs, together with the AAV rep coding region, provide for efficient excision and rescue from, and integration of a nucleotide sequence interposed between two flanking ITRs into a host cell genome. Sequences of certain AAV-associated ITRs are disclosed by Yan et al., J. Virol. (2005) vol. 79, pp. 364-379 which is herein incorporated by reference in its entirety. ITR sequences that find use herein may be full length, wild-type AAV ITRs or fragments thereof that retain functional capability, or may be sequence variants of full-length, wild-type AAV ITRs that are capable of functioning in cis as origins of replication. AAV ITRs useful in the recombinant AAV hC1-INH vectors of the embodiments provided herein may be derived from any known AAV serotype and, in certain embodiments, derived from the AAV2 serotype.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “transcription regulatory element” refers to nucleotide sequences of a gene involved in regulation of genetic transcription including a promoter, plus response elements, activator and enhancer sequences for binding of transcription factors to aid RNA polymerase binding and promote expression, and operator or silencer sequences to which repressor proteins bind to block RNA polymerase attachment and prevent expression. The term “liver specific transcription regulatory element” or “liver-specific transcription regulatory region” refers to a regulatory element or region that produces preferred gene expression specifically in the liver tissue.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

In one embodiment, the vector construct comprises a nucleic acid encoding a functionally active C1-INH protein. The C1-INH encoding sequence may be wild-type, codon optimized or a variant.

As used herein, “wild-type” SERPING1 (C1-INH-encoding gene) has the nucleotide sequence of SEQ ID NO: 1, or an allelic variant thereof.

As used herein, “wild-type” C1-INH protein has the mature amino acid sequence of SEQ ID NO: 2, or an allelic variant thereof.

The term “isolated” when used in relation to a nucleic acid molecule of the present disclosure typically refers to a nucleic acid sequence that is identified and separated from at least one contaminant nucleic acid with which it is ordinarily associated in its natural source. Isolated nucleic acid may be present in a form or setting that is different from that in which it is found in nature. Isolated nucleic acid molecules therefore are distinguished from the nucleic acid molecule as it exists in natural cells.

As used herein, the term “variant” refers to a polynucleotide (or polypeptide) having a sequence substantially similar to a reference polynucleotide (or polypeptide). Procedures for the introduction of nucleotide and amino acid changes in a polynucleotide, protein or polypeptide are known to the skilled artisan (see, e.g., Sambrook et al. (1989)). In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “identity,” “homology” and grammatical variations thereof, mean that two or more referenced entities are the same, when they are “aligned” sequences. Thus, by way of example, when two polypeptide sequences are identical, they have the same amino acid sequence, at least within the referenced region or portion. Where two polynucleotide sequences are identical, they have the same polynucleotide sequence, at least within the referenced region or portion. The identity can be over a defined area (region or domain) of the sequence. An “area” or “region” of identity refers to a portion of two or more referenced entities that are the same. Thus, where two protein or nucleic acid sequences are identical over one or more sequence areas or regions, they share identity within that region. An “aligned” sequence refers to multiple polynucleotide or protein (amino acid) sequences, often containing corrections for missing or additional bases or amino acids (gaps) as compared to a reference sequence. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology.

“Percent (%) nucleic acid sequence identity or homology or identical” is defined as the percentage of nucleotides in a candidate sequence that are identical with a reference sequence after aligning the respective sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN or Megalign (DNASTAR) software. Those skilled in the art can determine appropriate parameters for measuring alignment, including any algorithms needed to achieve maximal alignment over the full length of the sequences being compared.

“Percent (%) amino acid sequence identity or homology or identical” with respect to the C1-INH amino acid sequences identified herein is defined as the percentage of amino acid residues in a candidate sequence that are identical to the amino acid residues in a C1-INH polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN or Megalign (DNASTAR) software. Those skilled in the art can determine appropriate parameters for measuring alignment, including any algorithms needed to achieve maximal alignment over the full length of the sequences being compared.

“Codon optimization” or “codon optimized” refers to changes made in the nucleotide sequence so that it is more likely to be expressed at a relatively high level compared to the non-codon optimized sequence. It does not change the amino acid for which each codon encodes.

As used herein, an “intron” is broadly defined as a sequence of nucleotides that is removable by RNA splicing. “RNA splicing” means the excision of introns from a pre-mRNA to form a mature mRNA. Introns may be upstream, downstream, or within the coding region of a gene. Insertion of an intron into a nucleotide sequence can be accomplished by any method known in the art. The only limitation of where the intron is inserted is in consideration of the packaging limitations of the AAV virus particles (about 5 kbp).

In certain embodiments, the recombinant AAV vector construct comprises (a) a nucleic acid comprising an AAV2 5′ inverted terminal repeat (ITR) (which may or may not be modified as known in the art), (b) a liver-specific transcription regulatory region, (c) one or more introns, (d) a functional hC1-INH protein coding region, (e) a polyadenylation sequence, and (f) an AAV2 3′ ITR (which may or may not be modified as known in the art).

Other embodiments provided herein are directed to vector constructs encoding a functional C1-INH polypeptide, wherein the constructs comprise one or more of the individual elements of the above described constructs and combinations thereof, in one or more different orientation(s). Another embodiment provided herein is directed to the above described constructs in an opposite orientation. In another embodiment, provided are recombinant AAV virus particles comprising the herein described AAV vector constructs and their use for the treatment of HAE or deficiency in functional C1-INH in subjects.

An “AAV virion” or “AAV viral particle” or “AAV vector particle” or “AAV virus” refers to a viral particle composed of at least one AAV capsid protein and an encapsidated AAV vector construct as described herein. If the particle comprises a heterologous polynucleotide (i.e., a polynucleotide other than a wild-type AAV genome such as a transgene to be delivered to a mammalian cell), it is typically referred to as a “recombinant AAV vector particle” or simply an “AAV vector”. Production of AAV vector particles necessarily includes production of AAV vector genome, as such a vector genome is contained within an AAV vector particle. It is understood that reference to the polynucleotide AAV vector construct encapsulated within the vector particle, and replication thereof, refers to the AAV vector genome.

As used herein “therapeutic AAV virus” refers to an AAV virion, AAV viral particle, AAV vector particle, or AAV virus that comprises a heterologous polynucleotide that encodes a therapeutic protein such as the hC1-INH described herein. An “AAV vector construct” or “AAV vector genome” as used herein refers to a vector construct comprising a polynucleotide encoding a protein of interest (also called transgene) that are flanked by AAV terminal repeat sequences (ITRs) and operably linked to one or more expression control elements. Such AAV vector constructs can be replicated and packaged into infectious viral particles when present in a host cell that has been transfected with a vector encoding and expressing rep and cap gene products.

As used herein “therapeutic protein” refers to a polypeptide that has a biological activity that replaces or compensates for the loss or reduction of activity of an endogenous protein. For example, a functional C1 esterase inhibitor (C1-INH) is a therapeutic protein for hereditary angioedema (HAE).

“Hereditary angioedema (HAE)” as used herein refers to an inherited metabolic disease that is characterized by recurrent attacks or symptoms of subcutaneous and/or submucosal edema (swelling), particularly in the skin, gastrointestinal tract and respiratory tract due to activation of the complement pathway and/or contact activation pathway. The recurrent episodes of severe swelling can affect arms, legs, face, intestinal tract and airway which are painful, disfiguring and, sometimes, life threatening if they obstruct respiration. If left untreated, the condition has a 25% mortality rate.

Type I HAE and Type II HAE are caused by a deficiency of functional C1 esterase inhibitor (C1-INH) protein. Type I HAE is characterized by low expression levels of C1-INH. Type II HAE is characterized by normal or elevated expression levels of a non-functional C1-INH. Type III HAE is characterized by normal levels of functional C1-INH but a mutation in other genes such as Factor XII.

“C1 esterase inhibitor (C1-INH) deficiency” or a “deficiency in functional C1-INH” as used herein refers to an inherited condition caused by a deficiency of functional C1 esterase inhibitor (C1-INH) protein. This includes Type I and Type II HAE. The uninhibited activation of the complement and/or contact activation pathway due to insufficient levels of functional C1-INH results in unregulated cleavage of high molecular weight kininogen by kallikrein, leading to generation of excessive free bradykinin, a potent vasoactive peptide which increases capillary permeability and edema.

“Therapeutically effective for HAE” or “HAE therapy” as used herein refers to any therapeutic intervention of a subject having HAE that ameliorates HAE symptoms or reduces the frequency, duration or severity of acute HAE attacks, or reduces the amount of on-demand therapy (e.g. human C1-INH protein, kallikrein inhibitor, bradykinin antagonist, etc.) required to treat acute HAE attacks, or reduces the frequency with which on-demand therapy is administered to treat acute HAE attacks. “HAE gene therapy” as used herein refers to any therapeutic intervention of a subject having HAE that involves the replacement or restoration or increase of C1-INH activity through the delivery of one or more nucleic acid molecules to the cells of the subject that express functional C1-INH protein. In certain embodiments, HAE gene therapy refers to gene therapy involving an adeno associated viral (AAV) particle comprising a vector construct that expresses human C1-INH.

“Treat” or “treatment” as used herein refers to therapeutic treatment which refers to a treatment administered to a subject who exhibits signs or symptoms of pathology, i.e., HAE, for the purpose of diminishing or eliminating those signs or symptoms. The signs or symptoms can be biochemical, cellular, histological, functional, subjective or objective.

“Ameliorate” as used herein refers to the action of lessening the severity of symptoms, progression, or duration of a disease.

As used herein “stably treating” or “stable treatment” refers to using a therapeutic vector construct, AAV particle or cell administered to a subject where the subject stably expresses a therapeutic protein expressed by the vector construct, AAV particle or cell. Stably expressed therapeutic protein means that the protein is expressed for a clinically significant length of time.

“Clinically significant length of time” or “durability” as used herein with respect to HAE means expression at therapeutically effective levels for a length of time that has a meaningful impact on the on the quality of life of the subject. In certain embodiments a meaningful impact on the quality of life is demonstrated by the lack of a need to administer alternative therapies intravenously or subcutaneously. In certain embodiments clinically significant length of time is expression for at least six months, for at least eight months, for at least one year, for at least two years, for at least three years, for at least four years, for at least five years, for at least six years, for at least seven years, for at least eight years, for at least nine years, for at least ten years, or for the life of the subject. Preferably, therapeutically effective expression of functional C1-INH continues for at least five years.

As used herein, the term “effective amount” refers to an amount sufficient to effect beneficial or desirable biological and/or clinical results.

As used herein, a “subject” refers to a human patient being administered a treatment.

As used herein, a “normal laboratory reference range” or a “normal range” with respect to a particular test refers to the range or the interval of values that is deemed normal for a physiologic measurement on that particular test in healthy subjects without hereditary angioedema. The exact values of the upper end of the normal laboratory reference range (upper limit of normal, or ULN) and lower end of the range (lower limit of normal, or LLN) may vary from one laboratory to another depending on the test type, method and equipment in the laboratory performing the test. Laboratories report patient test results along with their normal laboratory reference ranges, so that clinicians can interpret the test result.

In general, a “pharmaceutically acceptable carrier” is one that is not toxic or unduly detrimental to cells. Exemplary pharmaceutically acceptable carriers include sterile, pyrogen-free water and sterile, pyrogen-free, phosphate buffered saline. Pharmaceutically acceptable carriers include physiologically acceptable carriers. The term “pharmaceutically acceptable carrier” includes any and all solvents, dispersion media, coatings, antibacterial and antifungal agents, isotonic and absorption delaying agents, and the like that are physiologically compatible.

### Recombinant AAV Vector Constructs

The recombinant vector construct of the disclosure may be used to produce rAAV particles by methods described herein, comprising providing to a suitable host cell the recombinant vector construct, together with Rep and Cap genes. The vector constructs described herein comprise a nucleic acid sequence that encodes a functional C1 esterase inhibitor (C1-INH). The recombinant vector construct may comprise a nucleic acid encoding functional human C1-INH operably linked to a heterologous expression control element, e.g. a promoter and/or enhancer; optionally an intron; and optionally a polyadenylation (polyA) signal. The heterologous expression control element may be a heterologous liver-specific transcription regulatory region, e.g., as described herein. When used to produce AAV particles, the recombinant vector construct may comprise (a) one or both of (i) an AAV 5′ inverted terminal repeat (ITR) sequence and (ii) an AAV 3′ ITR, (b) a heterologous liver-specific transcription regulatory region, and (c) a nucleic acid encoding a functional human C1 esterase inhibitor (hC1-INH), optionally wherein the AAV ITRs are AAV2 ITRs. Preferably, the nucleic acid encoding the functional hC1-INH is operably linked to liver-specific expression control elements. The vector construct may include additional expression control elements, for example: a promoter and/or enhancer; an intron; optionally an exon from the same gene as the intron; and a polyadenylation (polyA) signal. Such elements are further described herein. Preferably, the rAAV particles also comprise an AAV capsid with liver tropism, optionally an AAV5 type capsid.

In one embodiment, the vector construct comprises a nucleic acid encoding a functionally active hC1-INH protein. The hC1-INH encoding sequence may be wild-type, codon optimized, or a variant. One wild type SERPING1 gene has the nucleotide sequence of SEQ ID NO:1. One wild type hC1-INH has the mature amino sequence of SEQ ID NO: 2, amino acids 23 through 500.

The vector constructs described herein may comprise a nucleotide sequence that differs from wild type nucleotide sequence but still encodes a functional hC1-INH amino acid sequence at least 90%, 95%, 98% or 99% identical to amino acids 23 through 500 of SEQ ID NO: 2. According to this aspect, the nucleotide sequence may have substantial homology, e.g. at least 70%, 75%, 80%, 85%, 90%, 95%, 97%, 98% or 99% homology, to SEQ ID NO: 1 as long as it encodes a functional hC1-INH at least 90% identical (or 95%, 98% or 99% identical) to amino acids 23 through 500 of SEQ ID NO: 2. Preferably the nucleotide sequence is at least 97%, 98% or 99% identical to SEQ ID NO: 1. If the nucleic acid encodes a protein comprising a sequence having changes to any of the wild-type amino acids, the protein should still be a functional protein. A skilled person will appreciate that minor changes can be made to some of the amino acids of the protein without adversely affecting the function of the protein.

In one or more embodiments, the nucleic acid sequence encoding C1-INH is operably linked to one or more heterologous expression control elements. Preferably, the expression control element is a liver-specific expression control element. Examples of liver specific control elements include, but are not limited to, the mouse thyretin promoter (mTTR), the endogenous human factor VIII promoter (F8), human apolipoprotein E hepatic control region and active fragments thereof, human alpha-1-antitrypsin promoter (hAAT) and active fragments thereof, human alpha-1-microglobulin promoter and fragments thereof, human prothrombin promoter and active fragments thereof, human albumin minimal promoter, and mouse albumin promoter. Enhancers derived from liver-specific transcription factor binding sites are also contemplated, such as EBP, DBP, HNF1, HNF3, HNF4, HNF6, and EnhL.

In some embodiments, the vector constructs comprise a nucleic acid sequence encoding functional C1-INH that is operably linked to a heterologous liver-specific transcription regulatory region. The vector constructs may comprise other regulatory elements. In some embodiments, in the vector constructs described herein, the expression control elements include one or more of the following: a promoter and/or enhancer; optionally an intron; and a polyadenylation (polyA) signal.

The liver-specific transcription regulatory region may comprise one or more liver-specific expression control elements. In one or more embodiments, the liver-specific transcription regulatory region is a synthetic promoter sequence comprising portions of a human alpha-1-antitrypsin (hAAT) promoter, a hepatic control region (HCR) enhancer, and/or an apolipoprotein E (ApoE) enhancer.

In some embodiments, the vector construct comprises at least one or both of a 5′ inverted terminal repeat (ITR) of AAV and a 3′ AAV ITR, a promoter, a nucleic acid encoding functional C1-INH, and optionally a posttranscriptional regulatory element, where the promoter, the nucleic acid encoding C1-INH and the posttranscription regulatory element are located downstream of the 5′ AAV ITR and upstream of the 3′ AAV ITR. The vector construct can, for example, be used to produce high levels of C1-INH in a subject for therapeutic purposes.

In certain embodiments, the recombinant AAV vector construct comprises a nucleic acid comprising (a) an AAV2 5′ inverted terminal repeat (ITR) (which may or may not be modified as known in the art), (b) a liver-specific transcription regulatory region, a functional C1-INH protein coding region, (d) optionally one or more introns, including fragments of longer introns, (e) optionally an exon or fragment thereof, (f) a polyadenylation sequence, and (g) an AAV2 3′ ITR (which may or may not be modified as known in the art).

In some embodiments, the liver-specific transcription regulatory region comprises a shortened ApoE enhancer sequence (SEQ ID NO: 16) or a nucleotide sequence at least 80%, 85%, 90%, 95%, 97%, 98% or 99% identical thereto; a shortened hAAT promoter (SEQ ID NO: 3) or a 186 base human alpha anti-trypsin (hAAT) proximal promoter (SEQ ID NO: 15) or a nucleotide sequence at least 80%, 85%, 90%, 95%, 97%, 98% or 99% identical thereto, optionally including 42 bases of the 5′ untranslated region (UTR); one or more enhancers selected from the group consisting of (i) a 34 base human ApoE/C1 enhancer, (ii) a 32 base human AAT promoter distal X region, and (iii) 80 additional bases of distal element of the human AAT proximal promoter. In another embodiment, the liver-specific transcription regulatory region comprises an α-microglobulin enhancer sequence and the 186 base hAAT proximal promoter. Preferably the liver-specific transcription regulatory region comprises a fragment of an hAAT promoter and a fragment of an HCR enhancer/ApoE enhancer, e.g. a nucleotide sequence at least 80%, 85%, 90%, 95%, 97%, 98% or 99% identical to SEQ ID NO: 5.

Specific examples of liver specific promoters include LP1, HLP, HCR-hAAT, ApoE-hAAT, LSP, TBG and TTR. These promoters are described in more detail in the following references: LP1 (human ApoE HCR core sequence (192 bp) with human AAT promoter (255 bp)): Nathwani A. et al. Blood. 2006 Apr. 1; 107(7): 2653-2661; hybrid liver specific promoter (HLP) (human apolipoprotein E (ApoE) hepatic control region (HCR) fragment (34 bp) with modified human α-1-antitrypsin (aAT) promoter (217 bp)): McIntosh J. et al. Blood. 2013 Apr. 25; 121(17): 3335-3344; HCR-hAAT (ApoE-HCR (319 bp) with ApoE enhancer (1-4×154 bp) with human AAT promoter (408 bp) and including an Intron A (1.4 kbp) and 3′UTR (1.7 kbp)): Miao C H et al. Mol Ther. 2000; 1: 522-532; ApoE-hAAT: Okuyama T et al. Human Gene Therapy, 7, 637-645 (1996); LSP: Wang L et al. Proc Natl Acad Sci USA. 1999 Mar. 30; 96(7): 3906-3910, thyroxine binding globulin (TBG) promoter: Yan et al., Gene 506:289-294 (2012), and transthyretin (TTR) promoter: Costa et al., Mol. Cell. Biol. 8:81-90 (1988)

For example, De Simone et al. (EMBO Journal vol. 6 no. 9 pp. 2759-2766, 1987) describes a number of promoters derived from human α-1-antitrypsin promoter. For example, it characterizes the cis- and trans-acting elements required for liver-specific activity within the human AAT promoter from −1200 to +44. The human AAT promoter in HLP consists of the distal X element (32 bp) and the proximal A and B elements (185 bp). Frain et al. (MOL CELL BIO, March 1990, Vol. 10, No. 3, p. 991-999) describes a number of promoters derived from human albumin promoter. For example, it characterizes promoter and enhancer elements within the human albumin gene from −1022 to −1.

Dang et al. (J BIOL CHEM, Vol. 270, No. 38, Issue of September 22, pp. 22577-22585, 1995) describes the hepatic control region (HCR) of human apolipoprotein E gene (774 bp). Shachter et al. (J. Lipid Res. 1993. Vol. 34: pp 1699-1707) characterizes a liver-specific enhancer in the ApoE HCR (154 bp). These HCR fragments can be combined with other transcription regulatory elements such as the human AAT promoter or fragments thereof. Chow et al. (J Biol Chem. 1991 Oct. 5; 266(28):18927-33) characterizes the human prothrombin enhancer from −940 to −860 (80 bp). Rouet et al. (Vol. 267, No. 29, Issue of October 15, PP. 20765-20773, 1992; Nucleic Acids Res. 1995 Feb. 11; 23(3): 395-404; and Biochemical Journal Sep. 15, 1998, 334 (3) 577-584) characterize the sequence of the liver-specific human α-1-microglobulin/bikunin enhancer. U.S. Pat. No. 7,323,324 also describes human AAT promoter, human α-microglobulin/bikunen enhancers, human albumin promoter, and human prothrombin enhancers.

In some embodiments, the promoter comprises multiple copies of one or more of the enhancers identified above. In some embodiments, the promoter constructs comprise one or more of the individual enhancer elements described above and combinations thereof, in one or more different orientation(s).

Other embodiments provided herein are directed to vector constructs encoding a functional C1-INH polypeptide, wherein the constructs comprise one or more of the individual elements of the above described constructs and combinations thereof, in one or more different orientation(s). Another embodiment provided herein is directed to the above described constructs in an opposite orientation. In another embodiment, provided are recombinant AAV particles comprising the herein described vector constructs and their use for the treatment of HAE or C1-INH deficiency in subjects.

The size of the promoter can vary. Because of the limited packaging capacity of AAV, it is preferred to use a promoter that is small in size, but at the same time allows high level production of the protein(s) of interest in host cells. For example, in some embodiments the promoter is at most about 1.5 kb, at most about 1.4 kb, at most about 1.35 kb, at most about 1.3 kb, at most about 1.25 kb, at most about 1.2 kb, at most about 1.15 kb, at most about 1.1 kb, at most about 1.05 kb, at most about 1 kb, at most about 800 base pairs, at most about 600 base pairs, at most about 400 base pairs, at most about 200 base pairs, or at most about 100 base pairs.

Various additional regulatory elements can be used in the vector constructs, for example enhancers to further increase expression level of the protein of interest in a host cell, a polyadenylation signal, a ribosome binding sequence, and/or a consensus splice acceptor or splice donor site. In some embodiments, the regulatory element can facilitate maintenance of the recombinant DNA molecule extrachromosomally in a host cell and/or improve vector potency (e.g. scaffold/matrix attachment regions (S/MARs)). Such regulatory elements are well known in the art.

The vector constructs disclosed herein may include regulatory elements such as a transcription initiation region and/or a transcriptional termination region. Examples of a transcription termination region include, but are not limited to, polyadenylation signal sequences. Examples of polyadenylation signal sequences include, but are not limited to, human growth hormone (hGH) poly(A), bovine growth hormone (bGH) poly(A), SV40 late poly(A), rabbit beta-globin (rBG) poly(A), thymidine kinase (TK) poly(A) sequences, and any variants thereof. In some embodiments, the transcriptional termination region is located downstream of the posttranscriptional regulatory element. In some embodiments, the transcriptional termination region is a polyadenylation signal sequence. In some embodiments, the transcriptional termination region is bGH poly(A) sequence.

In some embodiments, the vector comprises one or more introns. The introns may facilitate processing of the RNA transcript in mammalian host cells, increase expression of the protein of interest and/or optimize packaging of the vector into AAV particles. Inclusion of an intron element may enhance expression compared with expression in the absence of the intron element (see e.g. Kurachi et al., 1995, J Biol Chem. 1995 Mar. 10; 270(10):5276-81). AAV vectors typically accept inserts of DNA having a defined size range which is generally about 4 kb to about 5.2 kb, or slightly more. However, there is no minimum size for packaging and small vector genomes package very efficiently. Introns and intron fragments fulfill this requirement while also enhancing expression. Thus, the disclosure contemplates the inclusion of hC1-INH intron sequences in the AAV vector or other introns or other DNA sequences in place of portions of a hC1-INH intron. Non-limiting examples of such an intron are a hemoglobin (0-globin) intron and/or hAAT (human alpha-1-antitrypsin) intron. In other embodiments, the intronic sequence is a composite hAAT/beta-globin intron. In some embodiments, the intron is a synthetic intron.

In some embodiments, the intron comprises a nucleotide sequence at least about 80%, 85%, 90%, 95%, 97%, 98% or 99% identical to any of SEQ ID NOs: 6, 61-69 or a fragment thereof. Preferably, the intron is at least 90%, 95%, 97%, 98% or 99% identical to SEQ ID NO: 6 or 64.

The location and size of the intron in the vector can vary. In some embodiments, the intron is located between the promoter and the sequence encoding the protein of interest. In some embodiments, the intron is located downstream of the sequence encoding the protein of interest. In some embodiments, the intron is located within the promoter. In some embodiments, the intron includes an enhancer element. In some embodiments, the intron is located within the sequence encoding the protein of interest, preferably between exons of the sequence encoding the protein of interest. In some embodiments, the intron may comprise all or a portion of a naturally occurring intron within the sequence encoding the protein of interest.

In any of the embodiments herein, the rAAV particle comprises a recombinant vector construct comprising a nucleotide sequence at least 90%, 95%, 97%, 98% or 99% identical to any one of SEQ NOs: 9, 20-36, 57 or 58. Preferably the recombinant vector construct is comprised within an AAV5 type capsid.

The AAV vector constructs provided herein in single strand form are less than about 7.0 kb in length, or are less than 6.5 kb in length, or are less than 6.4 kb in length, or are less than 6.3 kb in length, or are less than 6.2 kb in length, or are less than 6.0 kb in length, or are less than 5.8 kb in length, or are less than 5.6 kb in length, or are less than 5.5 kb in length, or are less than 5.4 kb in length, or are less than 5.3 kb in length, or are less than 5.2 kb in length or are less than 5.0 kb in length, or are less than 4.8 kb in length, or are less than 4.6 kb in length, or are less than 4.5 kb in length, or are less than 4.4 kb in length, or are less than 4.3 kb in length, or are less than 4.2 kb in length, or are less than 4.1 kb in length, or are less than 4.0 kb in length, or are less than 3.9 kb in length, or are less than 3.8 kb in length, or are less than 3.7 kb in length, or are less than 3.6 kb in length, or are less than 3.5 kb in length, or are less than 3.4 kb in length, or are less than 3.3 kb in length, or are less than 3.2 kb in length, or are less than 3.1 kb in length, or are less than 3.0 kb in length. The AAV vector constructs provided herein in single strand form range from about 5.0 kb to about 6.5 kb in length, or range from about 4.8 kb to about 5.2 k in length, or 4.8 kb to 5.3 kb in length, or range from about 4.9 kb to about 5.5 kb in length, or about 4.8 kb to about 6.0 kb in length, or about 5.0 kb to 6.2 kb in length or about 5.1 kb to about 6.3 kb in length, or about 5.2 kb to about 6.4 kb in length, or about 5.5 kb to about 6.5 kb in length, or range from about 4.0 kb to about 5.0 kb in length, or range from about 3.8 kb to about 4.8 k in length, or 3.6 kb to 4.6 kb in length, or range from about 3.4 kb to about 4.4 kb in length, or range from about 3.2 kb to about 4.2 kb in length, or range from about 3.0 kb to 4.0 kb in length, or range from about 3.5 kb to about 4.0 kb in length, or range from about 3.0 kb to about 3.5 kb in length, or range from about 4 kb to about 4.5 kb in length

When AAV vectors are produced from oversized recombinant vector constructs, they may lack a portion of the 5′ or 3′ ends of the recombinant vector construct. Because AAV is a single-stranded DNA virus, and packages either the sense or antisense strand, the sense strand in oversized AAV vectors lacks the 5′ AAV ITR and possibly portions of the 5′ end of the target protein-coding gene, and the antisense strand in oversized AAV vectors lacks the 3′ ITR and possibly portions of the 3′ end of the target protein-coding gene. A functional transgene is produced in oversized AAV vector infected cells by annealing of the sense and antisense truncated genomes within the target cell. Thus, in certain embodiments, the rAAV particles of the invention may comprise recombinant vector constructs that comprise at least one ITR, and a substantial portion of a nucleotide sequence encoding a functional hC1-INH, such as a fragment of SEQ ID NO: 1 that is greater than 50%, 60%, 70%, 80%, or 90% of the length of the full length nucleotide sequence. The rAAV particles of the invention may also comprise a substantial portion of any of any one of SEQ NOs: 10-12, e.g. a fragment that is greater than 50%, 60%, 70%, 80%, or 90% of the length of the nucleotide sequence set forth in any of SEQ ID NOs: 10-12.

Polynucleotides and polypeptides including modified forms can be made using various standard cloning, recombinant DNA technology, via cell expression or in vitro translation and chemical synthesis techniques known to those of skill in the art (Sambrook et al., Molecular Cloning: A Laboratory Manual, 2nd edition). Generation of the vector constructs can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

AAV vector constructs can be replicated and packaged into infectious AAV particles, preferably replication deficient AAV particles, when present in a host cell that has been transfected with a polynucleotide encoding and expressing rep and cap gene products.

### Recombinant AAV Particles and AAV Capsids

Production of AAV particles requires AAV “rep” and “cap” genes, which are genes encoding replication and encapsidation proteins, respectively. AAV rep and cap genes have been found in all AAV serotypes examined to date and are described herein and in the references cited. In wild-type AAV, the rep and cap genes are generally found adjacent to each other in the viral genome (i.e., they are “coupled” together as adjoining or overlapping transcriptional units), and they are generally conserved among AAV serotypes. AAV rep and cap genes are also individually and collectively referred to as “AAV packaging genes.” The AAV cap genes for use herein encode Cap proteins which are capable of packaging AAV vectors in the presence of rep and adeno helper function and are capable of binding target cellular receptors. In some embodiments, the AAV cap gene encodes a capsid protein having an amino acid sequence derived from a particular AAV serotype.

The AAV sequences employed for the production of AAV can be derived from the genome of any AAV serotype. Generally, the AAV serotypes have genomic sequences of significant homology at the amino acid and the nucleic acid levels, provide a similar set of genetic functions, produce virions which are essentially physically and functionally equivalent, and replicate and assemble by practically identical mechanisms. For the genomic sequence of AAV serotypes and a discussion of the genomic similarities. (See, e.g., GenBank Accession number U89790; GenBank Accession number J01901; GenBank Accession number AF043303; GenBank Accession number AF085716; Chiorini et al., J. Vir. (1997) vol. 71, pp. 6823-6833; Srivastava et al., J. Vir. (1983) vol. 45, pp. 555-564; Chiorini et al., J. Vir. (1999) vol. 73, pp. 1309-1319; Rutledge et al., J. Vir. (1998) vol. 72, pp. 309-319; and Wu et al., J. Vir. (2000) vol. 74, pp. 8635-8647).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins form the capsid. The assembly-activating protein (AAP) rapidly chaperones capsid assembly and prevents degradation of free capsid proteins (Grosse et al., J. Virol. 91(20):e01198-17, 2017). The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins, Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter. The ITRs employed in the vectors of the present embodiment may correspond to the same serotype as the associated cap genes or may differ. In one embodiment, the ITRs employed herein correspond to an AAV2 serotype and the cap genes correspond to an AAV5 serotype.

The AAV VP proteins are known to determine the cellular tropicity of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped AAV particles comprising the capsid proteins of a serotype (e.g., AAV1, 5 or 8) and the Rep and/or ITR sequences of another AAV serotype (e.g., AAV2). Such pseudotyped rAAV particles are a part of the present disclosure.

The AAV particles described herein (and the encoding AAV vector genomes) may comprise any of the capsid proteins described in WO 2018/022608 or PCT/US19/32097, incorporated by reference herein in its entirety for its disclosure of human and simian AAV capsids and their properties such as transduction efficiency, tissue tropism, glycan-binding, and resistance to neutralization by IVIG, including but not limited to any of the capsids in the sequence listing and variants thereof, e.g. with chimeric swapped variable regions and/or glycan binding sequences and/or GH loop.

In one embodiment, the AAV ITR sequences for use in the context of the present disclosure are derived from AAV1, AAV2, AAV4 and/or AAV6. Likewise, the Rep (e.g., Rep78 and Rep52) coding sequences are in one embodiment derived from AAV1, AAV2, AAV4 and/or AAV6. The sequences coding for the VP1, VP2, and VP3 capsid proteins for use in the context of the present disclosure may however be taken from any serotype, such as from AAV1, AAV2, AAV3, AAV4, AAV5, AAV6, AAV7, AAV8, AAV9, AAV10, AAV11 or AAV12, or from simian AAVs, including any of the capsid proteins described in WO 2018/022608 or PCT/US19/32097, or newly developed AAV-like particles obtained by e.g. capsid shuffling techniques and AAV capsid libraries, or any capsid at least 90% identical to any of SEQ ID NOs: 35-51.

For example, the amino acid sequences of various capsids are published. See, e.g., AAVRh.1/hu.14/AAV9 AAS99264.1 (SEQ ID NO: 35); AAVRh.8 SEQ97 of U.S. Pat. Pub. 2013/0045186 (SEQ ID NO: 36); AAVRh.10 SEQ81 of U.S. Pat. Pub. 2013/0045186 (SEQ ID NO: 37); AAVRh.74 SEQ 1 of Int′l. Pat. Pub. WO 2013/123503(SEQ ID NO: 38); AAV1 AAB_95452.1 (SEQ ID NO: 39); AAV2 YP_680426.1 (SEQ ID NO: 40); AAV3 NP_043941.1 (SEQ ID NO: 41); AAV3B AAB95452.1 (SEQ ID NO: 42); AAV4 NP_044927.1 (SEQ ID NO: 43); AAV5 YP_068409.1 (SEQ ID NO: 44); AAV6 AAB95450.1 (SEQ ID NO: 45); AAV7 YP_077178.1 (SEQ ID NO: 46); AAV8 YP_077180.1 (SEQ ID NO: 47); AAV10 AAT46337.1 (SEQ ID NO: 48); AAV11 AAT46339.1 (SEQ ID NO: 49); AAV12 ABI16639.1 (SEQ ID NO: 50); or AAV13 ABZ10812.1 (SEQ ID NO: 51).

In some embodiments, the AAV capsid comprises an amino acid sequence at least 85%, 90%, 95%, 98%, or 99% identical to any one of SEQ ID NOs: 35-51.

Preferably, the AAV capsid is an AAV capsid with liver tropism. In some instances, the AAV capsid with liver tropism excludes AAV8 and/or AAVHSC15. Preferably, the AAV capsid with liver tropism is an AAV5 type capsid, optionally at least 85%, 90%, 95%, 98%, or 99% identical to SEQ ID NO: 44.

Modified “AAV” sequences also can be used in the context of the present disclosure, e.g. for the production of AAV gene therapy vectors. Such modified sequences e.g. sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8 or AAV9 ITR, Rep, or VP, can be used in place of wild-type AAV ITR, Rep, or VP sequences.

In some embodiments, a nucleic acid sequence encoding an AAV capsid protein is operably linked to expression control sequences for expression in a specific cell type, such as Sf9 or HEK cells. Techniques known to one skilled in the art for expressing foreign genes in insect host cells or mammalian host cells can be used to practice the embodiment. Methodology for molecular engineering and expression of polypeptides in insect cells is described, for example, in Summers and Smith (1986) A Manual of Methods for Baculovirus Vectors and Insect Culture Procedures, Texas Agricultural Experimental Station Bull. No. 7555, College Station, Tex.; Luckow (1991) In Prokop et al., Cloning and Expression of Heterologous Genes in Insect Cells with Baculovirus Vectors' Recombinant DNA Technology and Applications, 97-152; King, L. A. and R. D. Possee (1992) The baculovirus expression system, Chapman and Hall, United Kingdom; O'Reilly, D. R., L. K. Miller, V. A. Luckow (1992) Baculovirus Expression Vectors: A Laboratory Manual, New York; W.H. Freeman and Richardson, C. D. (1995) Baculovirus Expression Protocols, Methods in Molecular Biology, volume 39; U.S. Pat. No. 4,745,051; US2003148506; and WO 03/074714, all of which are incorporated by reference in their entireties. A particularly suitable promoter for transcription of a nucleotide sequence encoding an AAV capsid protein is e.g. the polyhedron promoter. However, other promoters that are active in insect cells are known in the art, e.g. the p10, p35 or IE-1 promoters and further promoters described in the above references are also contemplated.

Use of insect cells for expression of heterologous proteins is well documented, as are methods of introducing nucleic acids, such as vectors, e.g., insect-cell compatible vectors, into such cells and methods of maintaining such cells in culture. (See, e.g., METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ.

Press (1994); Samulski et al., J. Vir. (1989) vol. 63, pp. 3822-3828; Kajigaya et al., Proc. Nat′l. Acad. Sci. USA (1991) vol. 88, pp. 4646-4650; Ruffing et al., J. Vir. (1992) vol. 66, pp. 6922-6930; Kirnbauer et al., Vir. (1996) vol. 219, pp. 37-44; Zhao et al., Vir. (2000) vol. 272, pp. 382-393; and U.S. Pat. No. 6,204,059). In some embodiments, the nucleic acid construct encoding AAV in insect cells is an insect cell-compatible vector. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In some embodiments, the vector is a baculovirus, a viral vector, or a plasmid. In one embodiment, the vector is a baculovirus, i.e. the construct is a baculoviral vector. Baculoviral vectors and methods for their use are described in the above cited references on molecular engineering of insect cells.

Methods of producing recombinant adeno-associated virus (AAV) particles comprising any of the AAV vector constructs provided herein comprise the steps of culturing a cell that has been transfected with any of the AAV vector constructs provided herein (in association with various AAV cap and rep genes) and recovering recombinant therapeutic AAV particles from the transfected cell or supernatant of the transfected cell.

The cells useful for recombinant AAV production provided herein include any cell type susceptible to baculovirus infection, including vertebrate or insect cells. For example, the insect cell line used can be from Spodoptera frugiperda, such as SF9, SF21, SF900+, drosophila cell lines, mosquito cell lines, e.g., Aedes albopictus derived cell lines, domestic silkworm cell lines, e.g. Bombyx mori cell lines, Trichoplusia ni cell lines such as High Five cells or Lepidoptera cell lines such as Ascalapha odorata cell lines; for example, High Five, Sf9, Se301, SeIZD2109, SeUCR1, Sf9, Sf900+, Sf21, BTI-TN-5B1-4, MG-1, Tn368, HzAm1, BM-N, Ha2302, Hz2E5, and Ao38. In another embodiment, mammalian cells such as HEK293, HeLa, CHO, NSO, SP2/0, PER.C6, Vero, RD, BHK, HT 1080, A549, Cos-7, ARPE-19, and MRC-5 can be used.

Baculoviruses are enveloped DNA viruses of arthropods, two members of which are well known expression vectors for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedrovirus (AcMNPV) or Bombyx mori nucleopolyhedrovirus (BmNPV) (Kato et al., (2010), Applied Microbiology and Biotechnology, vol. 85, Issue 3, pp 459-470).

Baculoviruses are commonly used for the infection of insect cells for the expression of recombinant proteins. In particular, expression of heterologous genes in insects can be accomplished as described in for instance U.S. Pat. No. 4,745,051; EP 127,839; EP 155,476; Vlak et al., (1988), Journal of General Virology, vol. 68, pp 765-776; Miller et al., (1988), Annual Review of Microbiology, vol. 42, pp 177-179; Carbonell et al., (1998), Gene, vol. 73, Issue 2, pp 409-418; Maeda et al., (1985), Nature, vol. 315, pp 592-594; Lebacq-Veheyden et al., (1988), Molecular and Cellular Biology, vol. 8, no. 8, pp 3129-3135; Smith et al., (1985), PNAS, vol. 82, pp 8404-8408; and Miyajima et al., (1987), Gene, vol. 58, pp 273-281. Numerous baculovirus strains and variants and corresponding permissive insect host cells that can be used for protein production are described in Luckow et al., (1988), Nature Biotechnology, vol. 6, pp 47-55; Maeda et al., (1985), Nature, vol. 315, pp 592-594; and McKenna et al., (1998), Journal of Invertebrate Pathology, vol. 71, Issue 1, pp 82-90.

Transient transfection of adherent HEK293 cells (Chahal et al., J. Virol. Meth. 196: 163-73 (2014)) and transfection of Sf9 cells, using the baculovirus expression vector system (BEVS) (Mietzsch et al., Hum. Gene Ther. 25: 212-22 (2014)), are two of the most commonly used methods to produce AAV vectors.

In some embodiments, the helper functions are provided by one or more helper plasmids or helper viruses comprising adenoviral or baculoviral helper genes. Non-limiting examples of the adenoviral or baculoviral helper genes include, but are not limited to, E1A, E1B, E2A, E4 and VA, which can provide helper functions to AAV packaging.

Helper viruses of AAV are known in the art and include, for example, viruses from the family Adenoviridae and the family Herpes viridae. Examples of helper viruses of AAV include, but are not limited to, SAdV-13 helper virus and SAdV-13-like helper virus described in US Publication No. 20110201088 (the disclosure of which is incorporated herein by reference), and helper vectors pHELP (Applied Viromics). A skilled artisan will appreciate that any helper virus or helper plasmid of AAV that can provide adequate helper function to AAV can be used herein.

In some embodiments, the AAV cap genes are present in a plasmid. The plasmid can further comprise an AAV rep gene which may or may not correspond to the same serotype as the cap genes. The cap genes and/or rep gene from any AAV serotype described herein (including, but not limited to, AAV1, AAV2, AAV4, AAV5, AAV6, AAV7, AAV8, AAV9, AAV10, AAV11, AAV12, AAV13 and any variants thereof) can be used to produce the recombinant AAV. In some embodiments, the AAV cap genes encode a capsid from serotype 1, serotype 2, serotype 4, serotype 5, serotype 6, serotype 7, serotype 8, serotype 9, serotype 10, serotype 11, serotype 12, serotype 13 or a variant thereof.

In some embodiments, the insect or mammalian cell can be transfected with the helper plasmid or helper virus, the vector construct and the plasmid encoding the AAV cap genes; and the recombinant AAV virus can be collected at various time points after co-transfection. For example, the recombinant AAV virus can be collected at about 12 hours, about 24 hours, about 36 hours, about 48 hours, about 72 hours, about 96 hours, about 120 hours, or a time between any of these two time points after the co-transfection.

Recombinant AAV particles can also be produced using any conventional methods known in the art suitable for producing infectious recombinant AAV. In some instances, a recombinant AAV can be produced by using an insect or mammalian cell that stably expresses some of the necessary components for AAV particle production. For example, a plasmid (or multiple plasmids) comprising AAV rep and cap genes, and a selectable marker, such as a neomycin resistance gene, can be integrated into the genome of the cell. The insect or mammalian cell can then be co-infected with a helper virus (e.g., adenovirus or baculovirus providing the helper functions) and the viral vector construct comprising the 5′ and 3′ AAV ITR (and the nucleotide sequence encoding the heterologous protein, if desired). The advantages of this method are that the cells are selectable and are suitable for large-scale production of the recombinant AAV particle. As another non-limiting example, adenovirus or baculovirus rather than plasmids can be used to introduce rep and cap genes into packaging cells. As yet another non-limiting example, both the viral vector construct containing the 5′ and 3′ AAV ITRs and the rep-cap genes can be stably integrated into the DNA of producer cells, and the helper functions can be provided by a wild-type adenovirus to produce the recombinant AAV.

Provided herein are methods for the production of a AAV particle, useful as a gene delivery vector, the method comprising the steps of:
(a) providing a cell permissive for AAV replication (e.g. an insect cell or mammalian cell) with one or more nucleic acid constructs comprising:
   (i) a nucleic acid molecule (recombinant vector construct) provided herein that has at least one (two) flanking AAV inverted terminal repeat nucleotide sequence;
   (ii) a nucleotide sequence encoding one or more AAV Rep proteins which is operably linked to a promoter that is capable of driving expression of the Rep protein(s) in the cell;
   (iii) a nucleotide sequence encoding one or more AAV capsid proteins which is operably linked to a promoter that is capable of driving expression of the capsid protein(s) in the cell;
   (iv) and optionally AAP and MAAP contained in the VP2/3 mRNA
(b) culturing the cell defined in (a) under conditions permitting expression of the Rep and the capsid proteins; and,
optionally (c) recovering the AAV particle, and
optionally (d) purifying the AAV particle. For example, the recombinant vector construct of (i) comprises (1) a 5′ and 3′ AAV ITR, (2) a heterologous liver-specific transcription regulatory region, and (3) a nucleic acid encoding a functional human C1 Esterase Inhibitor (hC1-INH).

Typically then, a method provided herein for producing a AAV gene delivery vector comprises: providing to a cell permissive for AAV replication (a) a nucleotide sequence encoding a template for producing vector genome, e.g. vector construct of the present disclosure (as described in detail herein); (b) nucleotide sequences sufficient for replication of the template to produce a vector genome (the first expression cassette defined above); (c) nucleotide sequences sufficient to package the vector genome into an AAV capsid (the second expression cassette defined above), under conditions sufficient for replication and packaging of the vector genome into the AAV capsid, whereby AAV particles comprising the vector genome encapsidated within the AAV capsid are produced in the cell.

Production methods may further comprise the step of affinity-purification of the rAAV particles comprising the recombinant AAV vector construct using an anti-AAV antibody, in one embodiment an immobilized antibody. In another embodiment, the anti-AAV antibody is a monoclonal antibody. One antibody for use herein is a single chain camelid antibody or a fragment thereof as e.g. obtainable from camels or llamas (see e.g. Muyldermans, 2001, Biotechnol. 74: 277-302). The antibody for affinity-purification of rAAV is an antibody that specifically binds an epitope on an AAV capsid protein, whereby in one embodiment the epitope is an epitope that is present on capsid protein of more than one AAV serotype. For example, the antibody may be raised or selected on the basis of specific binding to AAV5 capsid but at the same time also it may also bind to AAV1, AAV2, AAV3, AAV6, AAV8 or AAV9 capsids.

Generally, vector genome and capsid (cp) titers may be evaluated in any way that is suitable for measuring the respective vg and capsids. For example, quantitative polymerase chain reaction (qPCR) may be used to measure vg titers and enzyme-linked immunosorbent assay (ELISA) may be used to measure Cp titer. Alternatively, SEC (size-exclusion chromatography)-HPLC may be used to measure the vg and cp titers. In addition, RP (reverse phase)-HPLC assay may be used to evaluate the potential impact of process parameters on VP ratios.

qPCR may be used for vg quantification by quantitative polymerase chain reaction (qPCR) using a standard qPCR system, such as an Applied Biosystems 7500 Fast Real-Time PCR system. Alternatively, digital droplet PCR (ddPCR) may be used for Vg quantification. Primers and probes may be designed to target the DNA of the AAV, allowing its quantification as it accumulates during PCR. Examples of ddPCR are described in Pasi, K. John, et al. “Multiyear Follow-Up of AAV5-hFVII-SQ Gene Therapy for Hemophilia A.” New England Journal of Medicine 382.1 (2020): 29-40; Regan, John F., et al. “A Rapid Molecular Approach for Chromosomal Phasing.” PloS one 10.3 (2015): e01 18270; and Furuta-Hanawa, Birei, Teruhide Yamaguchi, and Eriko Uchida. “Two-Dimensional Droplet Digital PCR as a Tool for Titration and Integrity Evaluation of Recombinant Adeno-Associated Viral Vectors” Human gene therapy methods 30.4 (2019): 127-136. Other systems for vg quantification include SEC, SEC-HPLC, and size exchange chromatography multi-angle light scattering, all of which are described in WO 2021/062164, which is incorporated in its entirety by reference.

The capsid ELISA (cp-ELISA) assay measures intact capsids using, e.g., the AAV5 Capsid ELISA method and may utilize a commercially-available kit (for example, Progen PRAAV5). This kit ELISA employs a monoclonal antibody specific for a conformational epitope on assembled AAV5 or other capsids. Capsids can be captured on a plate-bound monoclonal antibody, followed by subsequent binding of a detection antibody. The assay signal may be generated by addition of conjugated streptavidin peroxidase followed by addition of colorimetric TMB substrate solution, and sulfuric acid to end the reaction. The titers of test samples are interpolated from a four-parameter calibration curve of the target capsid standard. Another system for quantifying capsid titers is SEC-MALS, which are described in WO 2021/062164.

### Pharmaceutical Formulations

In one embodiment, provided is a pharmaceutical composition comprising a rAAV particle or population of rAAV particles as disclosed herein and a pharmaceutically acceptable diluent, excipient, carrier and/or other medicinal agent, pharmaceutical agent or adjuvant, etc.

By “pharmaceutically acceptable” it is meant a material that is not biologically or otherwise undesirable, i.e., the material may be administered to a subject without causing any undesirable biological effects. Thus, such a pharmaceutical composition may be used, for example, in transfection of a cell ex vivo or in administering a viral particle or cell directly to a subject.

A carrier may be suitable for parenteral administration, which includes intravenous, intraperitoneal or intramuscular administration. Alternatively, the carrier may be suitable for sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions provided herein is contemplated.

In certain embodiments, the pharmaceutical formulations provided herein are liquid formulations that comprise recombinant AAV particles comprising any of the vector constructs disclosed herein. The concentration of recombinant AAV virions in the formulation may vary. In certain embodiments, the concentration of recombinant AAV particle in the formulation may range from 1×10¹³ to about 1×10¹⁴ vg/ml, for example, 6×10¹³ vg/ml.

In other embodiments, the AAV particle pharmaceutical formulation provided herein comprises one or more sterile pharmaceutically acceptable excipients to provide the formulation with advantageous properties for storage and/or administration to subjects for the treatment of the genetic disorder. In certain embodiments, the pharmaceutical formulations provided herein are capable of being stored at less than about −60° C. (minus 60 degrees centigrade), −40° C., or −20° C. for a period of at least 6 months, 1.5 years, or 2 years, with no appreciable change in stability. In addition, the pharmaceutical formulations provided herein are stable under suitable accelerated storage conditions. Examples of stressed conditions include at about 25° C. and about 60% humidity for a time period of, e.g., 6, 9, 12, 18 and/or 24 months, or (for drug substances intended for storage in a freezer) at about −20° C. for a time period of, e.g. 12 months. Examples of accelerated conditions include at about 2° C. to about 8° C. for a time period of, e.g., 6, 9, 12, 18 and/or 24 months, or (for drug substances intended for storage in a freezer) at about −20° C. for a time period of, e.g. 12 months. See, e.g., FDA Guidance for Industry: Stability Testing of New Drug Substances and Products, November 2003.

In this regard, the term “stable” means that the recombinant AAV particle present in the formulation essentially retains its physical stability, chemical stability and/or biological activity during storage. In certain embodiments, the recombinant AAV particle present in the pharmaceutical formulation retains at least about 80% of its vg/ml (or at least about 80% of its infectious rAAV particles) in a human patient during storage for a determined period of time at −65° C., in other embodiments at least about 85%, 90%, 95%, 98% or 99% of its vg/ml, or alternatively infectious rAAV particles, in a human subject.

In certain aspects, the formulation comprising recombinant AAV particle further comprises one or more buffering agents. For example, citrate, phosphate, tris(hydroxymethyl)aminomethane (Tris) or other buffers are well known in the art. In another embodiment, the recombinant AAV particle formulation provided herein may comprise one or more isotonicity agents, such as sodium chloride. Other buffering agents and isotonicity agents known in the art are suitable and may be routinely employed for use in the formulations provided herein.

In another embodiment, the recombinant AAV particle formulations provided herein may comprise one or more bulking agents, including cryoprotective agents. Exemplary bulking agents include without limitation mannitol, sucrose, dextran, lactose, trehalose, and povidone (PVP K24).

In yet another embodiment, the recombinant AAV particle formulations provided herein may comprise one or more surfactants, which may be non-ionic surfactants. Exemplary surfactants include ionic surfactants, non-ionic surfactants, and combinations thereof. For example, the surfactant can be, without limitation, TWEEN 80 (also known as polysorbate 80, or its chemical name polyoxyethylene sorbitan monooleate), sodium dodecylsulfate, sodium stearate, ammonium lauryl sulfate, TRITON AG 98 (Rhone-Poulenc), poloxamer 407, poloxamer 188 and the like, and combinations thereof.

The recombinant AAV particle formulations provided herein are stable and can be stored for extended periods of time without an unacceptable change in quality, potency, or purity. In one aspect, the formulation is stable at a temperature of about 5° C. (e.g., 2° C. to 8° C.) for at least 1 month, for example, at least 1 month, at least 3 months, at least 6 months, at least 12 months, at least 18 months, at least 24 months, or more. In another embodiment, the formulation is stable at a temperature of less than or equal to about −20° C. for at least 6 months, for example, at least 6 months, at least 12 months, at least 18 months, at least 24 months, at least 36 months, or more. In another embodiment, the formulation is stable at a temperature of less than or equal to about −40° C. for at least 6 months, for example, at least 6 months, at least 12 months, at least 18 months, at least 24 months, at least 36 months, or more. In another embodiment, the formulation is stable at a temperature of less than or equal to about −60° C. for at least 6 months, for example, at least 6 months, at least 12 months, at least 18 months, at least 24 months, at least 36 months, or more. For example, the formulation is stable at a temperature of less than or equal to about −20° C., −40° C., or −60° C. for 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, 72 months, 73 months, 74 months, 75 months, 76 months, 77 months, 78 months, 79 months, 80 months, 81 months, 82 months, 83 months, 84 months, 85 months, 86 months, 87 months, 88 months, 89 months, 90 months, 91 months, 92 months, 93 months, 94 months, 95 months, 96 months, 97 months, 98 months, 99 months, 100 months, 101 months, 102 months, 103 months, 104 months, 105 months, 106 months, 107 months, 108 months, 109 months, 110 months, 111 months, 112 months, 113 months, 114 months, 115 months, 116 months, 117 months, 118 months, 119 months, or 120 months.

In yet another aspect, the disclosure provides a pharmaceutical composition comprising rAAV particle at a concentration of at least 1E13 vg/ml, to about 1E14 vg/ml, a buffering agent, an isotonicity agent, a cryopreservative agent and a surfactant which is stable during storage at about −60° C. (minus sixty degrees centigrade) or less for at least about 1 year, 1.5 years, or 2 years. In some embodiments, the buffer is a Tris buffer. In some embodiments, the cryopreservative agent is a sugar, for example, trehalose or a suitable hydrate thereof. In some embodiments, the surfactant is a poloxamer, e.g., poloxamer 188, or alternatively a polysorbate at a concentration of less than 0.2% w/v, or less than 0.15% w/v, for example, about 0.1% w/v.

In some embodiments, the pharmaceutical composition is aqueous and comprises rAAV particle at a concentration of at least 6E13 vg/ml, Tris buffer at a concentration of about 10 to about 30 mM, sodium chloride at a concentration of about 100 mM to about 165 mM, trehalose at a concentration of about 2 to about 3 wt %, and a poloxamer or polysorbate at a concentration of about 0.05% to about 0.15% w/v.

In some embodiments, the Tris is at a concentration of about 10 to about 50 mM, about 10 to about 30 mM, or about 15 to about 25 mM. In some embodiments, the sodium chloride is at a concentration of about 100 to about 140 mM, or about 110 to about 130 mM. Optionally, the trehalose is at a concentration of about 2 to about 3 wt %, or about 2.3 to about 2.7 wt %. Optionally, the poloxamer is poloxamer 188 at a concentration of about 0.05% to 0.15% w/v, optionally about 0.1% w/v.

In some embodiments, the pharmaceutical composition comprises rAAV particle at a concentration of about 6E13 vg/ml, 20 mM Tris, 120 mM sodium chloride, 2.5 wt % trehalose (dihydrate), and 0.1% w/v poloxamer 188.

Preferably the pharmaceutical composition is a liquid aqueous solution and is for storage at freezing temperature. In different embodiments, the pharmaceutical composition is stored at a temperature of ≤−60° C./−60 degrees Celsius (° C.) or less, where the stability or potency of the pharmaceutical composition is at least substantially maintained at the storage temperature. In other embodiments, the pharmaceutical composition is stored at a temperature of ≤−20° C. or about −20° C. or less without an unacceptable change in quality, potency, or purity. In different examples, the pharmaceutical composition is stored at a temperature of −20° C., −21° C., −22° C., −23° C., −24° C., −25° C., −26° C., −27° C., −28° C., −29° C., −30° C., −31° C., −32° C., −33° C., −34° C., −35° C., −36° C., −37° C., −38° C., −39° C., −40° C., −41° C., −42° C., −43° C., −44° C., −45° C., −46° C., −47° C., −48° C., −49° C., −50° C., −51° C., −52° C., −53° C., −54° C., −55° C., −56° C., −57° C., −58° C., −59° C., −60° C., −61° C., −62° C., −63° C., −64° C., −65° C., −66° C., −67° C., −68° C., −69° C., −70° C., −71° C., −72° C., −73° C., −74° C., −75° C., −76° C., −77° C., −78° C., −79° C., −80° C., −81° C., −82° C., −83° C., −84° C., −85° C., −86° C., −87° C., −88° C., −89° C., −90° C., −91° C., −92° C., −93° C., −94° C., −95° C., −96° C., −97° C., −98° C., −99° C., or −100° C. without an unacceptable change in quality, potency, or purity. In any of these embodiments, the composition is for use in intravenous administration of rAAV particle to a patient with hereditary angioedema.

### Methods of Treatment

In any of the embodiments, the subject has hereditary angioedema (HAE), optionally Type II or Type II HAE. In some embodiments, prior to administration of rAAV particles as described herein, the subject has a plasma or serum C1-INH levels about 50% of the lower limit of normal (LLN), or lower, and/or a C4 complement level below normal range. In some instances, the subject has a plasma or serum C1-INH level 10%, 20%, 30% or 40% of the LLN. The C1-INH levels may be measured by known functional or antigenic assays, preferably a functional chromogenic assay. Examples include a chromogenic assay (Technochrom C1 INH) and a C1 INH C1s complex formation assay (C1 Inhibitor Plus MicroVue Quidel) as described in Gompels et al., Ann Clin Biochem. 44[Pt 1]:75 8 (2007). Antigenic levels of C1 INH can be measured using nephelometry and Liquid Chromatography Mass Spectrometry (LC MS/MS) methods.

In some embodiments, prior to rAAV particle administration, the subject is a patient that is at least 1 years old. For example, the patient is a pediatric patient. In some embodiments, the subject is 18 years or more years old. In some embodiments, the subject is an adult. In some embodiments, the subject is a male. In some embodiments, the subject is a female, e.g. a nonpregnant female. In some embodiments, the subject is a juvenile, for example, age 12 to 18, or age 6 to 12, or age 6 to 18, or age 0 to 6.

In any of the embodiments, the subject may have a mutation in an endogenous SERPING1 gene encoding C1-INH, optionally detected by PCR followed by genome sequencing or restriction fragment length polymorphism (RFLP) analysis.

In some embodiments, prior to rAAV particle administration, the subject may have suffered HAE attacks at a frequency of at least 1 attack per month on average for at least 6 months. In some embodiments, the subject has received on-demand HAE-specific medication for the treatment of acute attacks and/or prophylactic HAE-specific medication for at least 6 months prior to rAAV particle administration.

As described in Craig, Timothy, et al. “WAO guideline for the management of hereditary angioedema.” World Allergy Organization Journal 5.12 (2012): 182-199, one unit of pdC1-INH is equivalent to the C1-INH content of one milliliter of human plasma (270 milligrams (mg)/Liter (L). So, 100% functional is equal to 1 IU, which is equal to 270 microgram (μg)/milliliter (mL). To this extent, the normal range of functional C1-INH(f) is 70-130% or 160-320 μg/mL of total C1-INH in plasma. In some embodiments, prior to rAAV particle administration, the subject is a patient having an abnormal C1-INH(f) value that is outside the normal range of functional C1-INH(f). For example, the abnormal C1-INH(f) value is outside the range of 70-130%. In another example, the abnormal C1-INH(f) value is outside the range of 160-320 μg/mL.

In some embodiments, prior to rAAV particle administration, the subject may have suffered low burden, or mild HAE, e.g. patients experiencing about 2 or less HAE attacks per year. In some embodiments, prior to rAAV particle administration, the subject may have suffered moderate HAE, e.g. patients experiencing about 3 to about 12 HAE attacks per year. In some embodiments, prior to rAAV particle administration, the subject may have suffered from severe HAE, e.g. patients experiencing about 13 or more HAE attacks per year.

Treatments for acute HAE attacks (on-demand HAE-specific medication) include plasma-derived C1-INH (e.g., BERINERT); recombinant C1INH (e.g., RUCONEST); bradykinin B2 receptor antagonists such as FIRAZYR (icatibant); plasma kallikrein inhibitors such as KALBITOR (ecallantide). Long-term prophylactic therapy against HAE attacks (prophylactic HAE-specific medication) includes plasma-derived C1-INH (e.g., CINRYZE, HAEGARDA); recombinant C1-INH; plasma kallikrein inhibitor such as ORLADEYO (berotralstat), anti-kallikrein antibody, such as TAKHZYRO (lanadelumab), or androgens such as danazol, oxandrolone, and stanozolol.

In some embodiments, the subject has not received steroids at least 30 days prior to said administration. In some embodiments, the subject has not used any androgens or attenuated androgens in the last one year prior to rAAV particle administration, or has not had one year of cumulative androgen or attenuated androgen use over the last four years.

In some embodiments, the subject does not have detectable anti-AAV capsid antibody in blood when the rAAV particles are administered (e.g., is not AAV5 seropositive). Anti-AAV neutralizing antibodies are undesirable because they may block cell transduction or otherwise reduce the overall efficiency of the treatment.

In some embodiments, the subject does not have clinically significant liver disease prior to rAAV particle administration. In some embodiments, the subject does not have clinically significant liver disease prior to said administration. For example, the subject does not have a Grade 3 or higher liver fibrosis, optionally as diagnosed by transient elastography or a prior liver biopsy (Grade 3 or 4 as rated on a scale of 0-4); or optionally an equivalent grade of fibrosis as diagnosed through a positive serologic marker test of liver fibrosis (ELF with a test score ≥7.2). In some embodiments, the subject does not have an elevation in any of ALT (alanine transaminase), AST (aspartate aminotransferase), GGT (gamma-glutamyltransferase), bilirubin or ALP (alkaline phosphatase) to more than 1.25 times the upper limit of normal (ULN), or the international normalized ratio being equal to or greater than 1.2. Preferably the subject does not have an elevation in AST and/or ALT of more than 1.25 times ULN.

In some embodiments, the subject does not have (1) evidence of an active or chronic infection, including severe acute respiratory syndrome coronavirus 2 (SARS-CoV-2), or immunosuppressive disorder; (2) active malignancy, autoimmune, metabolic, hematologic, cardiac or renal disease; (3) substance use disorder, major depressive disorder, psychosis, or bipolar disorder; (4) contraindication to glucocorticoids; or history of clinically significant venous thrombosis or arterial thrombosis. In some embodiments, the subject does not have prior infection with hepatitis B or C. In some embodiments, the subject does not have serum creatinine greater than or equal to 1.5 mg/dL.

Prior to infusion of the rAAV particles, subjects are evaluated for: (1) baseline physical examination; (2) baseline clinical laboratory tests, including (a) plasma C1-ING(f) levels, (b) C4 levels, and (c) liver enzyme tests, including ALT, AST, GGT, total bilirubin, alkaline phosphatase and INR; (d) and baseline AAV5 antibody detection; (3) measures of health-related quality of life (HRQoL), e.g. Angioedema Quality of Life Questionnaire (AE-QOL) score, Angioedema Control Test (AECT) score, Treatment Satisfaction Questionnaire for Medication (TSQM-9), EuroQoL-5D-5L (EQ-5D-5L) score, and Patient Global Impression of Severity (PSI-S) score; (4) baseline levels of other parameters monitored during the study; and (5) SEPRING-1 genotyping, if permitted.

In the methods of the disclosure, the rAAV particle is administered intravenously in a single dose administration. In some embodiments, the vector construct or recombinant AAV particle is administered by intravenous injection either as a single bolus or via infusion over a prolonged time period, which may be at least about 1, 5, 10, 15, 30, 45, 60, 75, 90, 120, 150, 180, 210 or 240 minutes, or more. In some embodiments, subjects receive a prophylactic short term IV injection of 1000 IU plasma derived or recombinant C1-INH (independent from previous HAE long term prophylaxis treatment), as a precautionary safety measure to ensure normal levels of C1 INH during infusion. In some circumstances, a second dose may be administered.

In some embodiments, the rAAV particle is administered at a dose ranging from about 2E13 to about 6E14 vector genomes per kilogram body weight of the subject (vg/kg), for example, a dose of about 2E13 vg/kg, or a dose of about 6E13 vg/kg, or a dose of about 2E14 vg/kg, or a dose of about 4E14 vg/kg, or a dose of about 6E14 vg/kg.

After infusion of the rAAV particles, the methods may further comprise the step of monitoring various parameters, e.g. measuring the parameters on a weekly basis. Measuring can alternatively occur every 1, 2, 3, 4, 5 or 6 days or every week or every two weeks or every three weeks or every month. Parameters may be monitored through Week 24, 48, 96 or longer. The methods may include measuring plasma or serum functional or antigenic C1-INH (C1-INH(f)) of the subject. For example, plasma C1-INH(f) levels are measured, and an increase from baseline in mean plasma C1-INH(f) levels at Week 8, 12 and 24 post-infusion is observed.

The methods of the disclosure may result in clinically significant increase of plasma or serum functional C1-INH (C1-INH(f)) levels (e.g. increase in mean g/mL plasma C1-INH levels, or percentage increase). For example, the plasma C1-INH level of said subject is increased by at least about 20 μg/mL or more by 8 weeks after said administration, or at least about 20 μg/mL or more at 24, 48, or 96 weeks, or at six months, or at one year, or at 2, 3, 4 or 5 years after said administration. For example, the plasma C1-INH(f) level of said subject is increased greater than or equal to about 10% (at least about 10%) or more by 8 weeks after said administration, or greater than or equal to 10% or more at 24, 48, or 96 weeks, or at six months, or at one year or at 2, 3, 4 or 5 years after said administration. For example, the plasma C1-INH level of said subject is increased 0.4 IU/ml, or 1 IU/ml or higher, or to about 16 mg/dL or higher by 8 weeks after said administration, or increased 0.4 IU/ml, or 1 IU/ml or higher, or to about 16 mg/dL or higher at 24, 48, or 96 weeks, or at six months, or at one year, or at 2, 3, 4 or 5 years after said administration. Preferably the administered dose of rAAV particles maintains an increased plasma level for a period of at least about six months, about one year, or 2, 3, 4 or 5 years.

In some embodiments, the dose may be effective to reduce the number or severity of acute HAE attacks of the subject, preferably over a period of at least about six months. For example, the dose is effective to reduce the number of HAE attacks to fewer than one HAE attack a month on average; for example, less than 5, less than 4, less than 3, or less than 2 HAE attacks over a period of six months. For example, the dose is effective to reduce the number of HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of at least 6 months. For example, the dose is effective to reduce the number of HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of at least one year.

In some embodiments, the dose may be effective to render patients attack-free, e.g. at least 10%, 15%, 20%, 25%, 30%, 35%, 40%, 45%, 50%, 55%, 60%, 65%, 70%, 75%, 80%, 85%, 90%, or 95% of treated patients are attack-free at 4 months. In some embodiments, at least 10%, 15%, 20%, 25%, 30%, 35%, 40%, 45%, 50%, 55%, 60%, 65%, 70%, 75%, 80%, 85%, 90%, or 95% of treated patients are attack-free for at least 6 months. In some embodiments, at least 10%, 15%, 20%, 25%, 30%, 35%, 40%, 45%, 50%, 55%, 60%, 65%, 70%, 75%, 80%, 85%, 90%, or 95% of treated patients are attack-free for at least one year.

In some embodiments, the dose is effective to reduce the number of moderate and severe acute HAE attacks of the subject, preferably over a period of at least about six months. In some embodiments, the dose is effective to reduce the number of high morbidity acute HAE attacks of the subject, preferably over a period of at least about six months. In some embodiments, the dose is effective to reduce the number of moderate to severe acute HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of at least six months. In some embodiments, the dose is effective to reduce the number of moderate to severe acute HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of at least one year. In some embodiments, the dose is effective to reduce the number of high morbidity (severe) acute HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of at least six months. In some embodiments, the dose is effective to reduce the number of high morbidity (severe) acute HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of at least one year. In some embodiments, the reduction in HAE attacks is maintained for at least about one year, or 2, 3, 4 or 5 years. In some embodiments, the dose may be effective to render patients free of high morbidity (or severe) HAE attacks, e.g. at least 10%, 15%, 20%, 25%, 30%, 35%, 40%, 45%, 50%, 55%, 60%, 65%, 70%, 75%, 80%, 85%, 90%, or 95% of treated patients are free of high morbidity (or severe) HAE attacks at 4 months. In some embodiments, at least 10%, 15%, 20%, 25%, 30%, 35%, 40%, 45%, 50%, 55%, 60%, 65%, 70%, 75%, 80%, 85%, 90%, or 95% of treated patients are free of high morbidity (or severe) HAE attacks for at least 6 months. In some embodiments, at least 10%, 15%, 20%, 25%, 30%, 35%, 40%, 45%, 50%, 55%, 60%, 65%, 70%, 75%, 80%, 85%, 90%, or 95% of treated patients are free of high morbidity (or severe) HAE attacks for at least one year

HAE attacks include symptoms or signs consistent with an attack in at least 1 of the following locations: (1) Peripheral angioedema: cutaneous swelling involving an extremity, the face, neck, torso, and/or genitourinary region; (2) Abdominal angioedema: abdominal pain, with or without abdominal distention, nausea, vomiting, or diarrhea; and/or (3) Laryngeal angioedema: stridor, dyspnea, difficulty speaking, difficulty swallowing, throat tightening, or swelling of the tongue, palate, uvula, or larynx. To be considered as a unique HAE attack that is distinct from the most recent previous HAE attack, the new symptoms must have occurred at least 24 hours after resolution of the symptoms from the prior attack.

A high morbidity HAE attack has at least one of the following characteristics: is severe, results in hospitalization (except hospitalization for observation <24 hours), is hemodynamically significant (systolic blood pressure <90, requires IV hydration, or associated with syncope or near syncope), or is laryngeal.

In some embodiments, the dose is effective to reduce the dose of or frequency of administration of HAE-specific therapy to the subject for acute HAE attacks (on-demand HAE-specific medication), on average, over a period of at least about 3, 6, 9 or 12 months. In some embodiments, the dose may be effective to reduce the dose of HAE-specific therapy for acute HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of at least about six months. In some embodiments, the dose may be effective to reduce the frequency of HAE-specific therapy for acute HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of at least about six months. In some embodiments, the dose may be effective to reduce the dose of HAE-specific therapy for acute HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of at least about one year. In some embodiments, the dose may be effective to reduce the frequency of HAE-specific therapy for acute HAE attacks by at least about 5%, 10%, 15%, 20%, 25%, 30%, 35%, 40%, 45%, 50%, 55%, 60%, 65%, 70%, 75%, 80%, 85%, 90%, 95% or 100% over a period of at least about one year. In some embodiments, the reduction is maintained for at least about one year, or 2, 3, 4 or 5 years.

In some embodiments, the dose is effective to reduce the dose of or frequency of administration of prophylactic HAE-specific medication to the subject, on average, over a period of at least about 3, 6, 9 or 12 months. In some embodiments, the dose may be effective to reduce the dose of prophylactic HAE-specific therapy by at least about 5%, 10%, 15%, 20%, 25%, 30%, 35%, 40%, 45%, 50%, 55%, 60%, 65%, 70%, 75%, 80%, 85%, 90%, 95% or 100% over a period of at least about six months. In some embodiments, the dose may be effective to reduce the dose of prophylactic HAE-specific therapy by at least about 5%, 10%, 15%, 20%, 25%, 30%, 35%, 40%, 45%, 50%, 55%, 60%, 65%, 70%, 75%, 80%, 85%, 90%, 95% or 100% over a period of at least about one year. In some embodiments, the reduction is maintained for at least about one year, or 2, 3, 4 or 5 years.

In some embodiments, the dose may be effective to eliminate prophylactic HAE-specific therapy in at least 10%, 15%, 20%, 25%, 30%, 35%, 40%, 45%, 50%, 55%, 60%, 65%, 70%, 75%, 80%, 85%, 90%, or 95% of treated patients over a period of at least about six months. In some embodiments, the dose may be effective to eliminate prophylactic HAE-specific therapy in at least 10%, 15%, 20%, 25%, 30%, 35%, 40%, 45%, 50%, 55%, 60%, 65%, 70%, 75%, 80%, 85%, 90%, or 95% of treated patients over a period of at least about one year.

The method may further comprise the step of monitoring health-related quality of life (HRQoL), e.g. as measured by Angioedema Control Test (AECT) score, Angioedema Quality of Life Questionnaire (AE-QOL) score, Treatment Satisfaction Questionnaire for Medication (TSQM-9), EuroQoL-5D-5L (EQ-5D-5L) score, Patient Global Impression of Severity (PSI-S) and/or Patient Global Impression of Change (PSI-C) scores. A clinically significant improvement in any of these parameters is observed, e.g. by Week 24, 48, or 96. In some embodiments, the dose is effective to improve health-related quality of life preferably over a period of at least about six months, optionally as measured by any one or more of Angioedema Quality of Life Questionnaire (AE-QOL) score, Angioedema Control Test (AECT) score, Treatment Satisfaction Questionnaire for Medication (TSQM-9), EuroQoL-5D-5L (EQ-5D-5L) score, or Patient Global Impression of Severity (PSI-S) score. In some embodiments, the dose may be effective to improve one or more of these measures of health-related quality of relief by at least about 5%, 10%, 15%, 20%, 25%, 30%, 35%, 40%, 45%, 50%, 55%, 60%, 65%, 70%, 75%, 80%, 85%, 90%, 95% or 100% over a period of at least about six months. In some embodiments, the dose may be effective to improve one or more of these measures of health-related quality of relief by at least about 5%, 10%, 15%, 20%, 25%, 30%, 35%, 40%, 45%, 50%, 55%, 60%, 65%, 70%, 75%, 80%, 85%, 90%, 95% or 100% over a period of at least about one year. In some embodiments, the improvement is maintained for at least about one year, or 2, 3, 4 or 5 years.

The methods of the disclosure provide administration of rAAV particles in a manner that is safe, e.g., no or low incidence of clinically significant treatment-emergent serious adverse events; no or low incidence of clinically significant changes in standard clinical laboratory values; no or low incidence of complement activation or hypersensitivity; no or low incidence of abnormalities in coagulation markers; no or low incidence of elevation to Grade 2 or higher or Grade 3 or higher of markers of hepatotoxicity such as AST and/or ALT (or if changes occur, most are transient or resolve after treatment with systemic immunosuppressant). The methods may also provide a reduced immune response against the AAV capsid. The methods may also provide improved blood biodistribution, or reduced vector shedding in urine, stool, semen, or saliva.

In an aspect of the disclosure, hepatotoxicity, e.g. as detected through transient hepatic transaminase enzyme elevations, may be reduced or avoided by prophylactic immunosuppression treatment or therapeutic immunosuppression treatment. According to these aspects, in addition to administration of a therapeutically effective amount of AAV virus, the subject may be treated either prophylactically, therapeutically, or both with a glucocorticoid or other immunosuppressant to prevent and/or treat any hepatotoxicity associated with administration of the AAV virus.

### Prophylactic Immunosuppression Treatment

The methods of disclosure may further comprise administering to the subject a prophylactically effective amount of a glucocorticoid to prevent hepatotoxicity, prior to detection of hepatotoxicity (e.g. as detected by ALT elevation above the upper limit of normal (ULN), or at least 2 times baseline ALT). In some embodiments, the prophylactically effective amount of immunosuppressant (e.g. glucocorticoid) is administered concurrent with administration of the rAAV particles of the invention. “Concurrent” as used herein means the same day, for example, or within one day or one week of (prior to or after) administration of the rAAV particles. In other embodiments, the administration of the prophylactically effective amount of immunosuppressant (e.g. glucocorticoid) begins after administration of the rAAV particles, e.g. starting at 3, 4, 5, 6, 7, 8, 9 or 10 weeks after administration of the rAAV particles, but prior to detection of hepatotoxicity.

The glucocorticoid or other immunosuppressant may be administered for a prophylactic treatment time period, e.g., for a time period of at least about 3 to 13 weeks (3, 4, 5, 6, 7, 8, 9, 10, 11, 12, 13 weeks), and is preferably followed by tapering period during which tapering amounts of the glucocorticoid or other immunosuppressant are administered, e.g., for a time period of about 2 to 4 weeks, or about 2, 3, or 4 weeks. For example, the prophylactically effective amount of the glucocorticoid is a prednisone-equivalent dose of from 10 mg/day to 40 mg/day for a time period of at least about 3 to 13 weeks (3, 4, 5, 6, 7, 8, 9, 10, 11, 12, 13), followed by tapering amounts of the glucocorticoid for a time period of about 2, 3 or 4 weeks. In some embodiments, the prophylactically effective amount of the glucocorticoid is administered for a time period of about 13 weeks, followed by tapering amounts of the glucocorticoid for a time period of about 3 weeks. For example, a prednisone equivalent is administered at a prednisone-equivalent dose of 40 mg/day concurrent with said administration for a time period of about 13 weeks, followed by tapering amounts of the prednisone equivalent for a time period of about 3 weeks (e.g., prednisone-equivalent dose of 30 mg/day for a week, 20 mg/day for a week, and 10 mg/day for a week).

In some embodiments, the subject is administered a 16-week prophylactic glucocorticoid course of a prednisone equivalent at a starting prednisone-equivalent dose of 40 mg/day, beginning on Day 1 a few hours pre-infusion of rAAV particles, for a time period of 13 weeks dosing at 40 mg/day, followed by a 3-week dose taper beginning at Week 14 (to a prednisone-equivalent dose of 30 mg/day for a week, 20 mg/day for a week, and 10 mg/day for a week). On the day of infusion, prophylactic glucocorticoids should be administered at a minimum 3 hours before rAAV particle infusion. ALT and AST levels are monitored weekly. If there is ALT elevation to greater than upper limit of normal (ULN) or greater than 2× baseline ALT value, during the first 12 weeks, adjustments to glucocorticoid dosing are based on clinical judgment, and liver enzymes may be monitored more frequently.

### Therapeutic Immunosuppression Treatment

Administration of an AAV particle of the present disclosure may, in some cases, result in an observable degree of hepatotoxicity. Hepatotoxicity may be measured by a variety of well-known and routinely used techniques for example, measuring concentrations of certain liver-associated enzyme(s) (e.g., alanine transaminase, ALT) in the bloodstream of a subject both prior to AAV administration (i.e., baseline) and after AAV administration. An observable increase in ALT concentration after AAV administration (as compared to prior to administration) is indicative of drug-induced hepatotoxicity. The methods of the disclosure may comprise administering to the subject a therapeutically effective amount of a glucocorticoid or other systemic immunosuppressant to treat hepatotoxicity, upon detection of hepatotoxicity.

Reactive immunosuppressant (e.g., glucocorticoid) therapy may be initiated after the prophylactic regimen is completed, or in response to mild ALT elevations that meet pre-specified criteria, or based on clinical judgment. In some embodiments, it is initiated if ALT is greater than the ULN or greater than 2× baseline in two consecutive assessments within 72 hours, or 3×ULN in two consecutive assessments within 48 hours. In some embodiments, the reactive immunosuppressive (e.g. glucocorticoid) regimen has a total duration of 8 weeks with 5 weeks of 40 mg/day prednisone-equivalent dosing, followed by a 3-week dose taper if ALT is both less than or equal to ULN and less than or equal to 2× baseline value. Liver enzymes are monitored weekly over 4 weeks in the period following discontinuation of reactive immunosuppression therapy, or more frequently if ALT values are above the ULN.

The methods of disclosure may further comprise the step of (a) determining a baseline level of a marker of hepatotoxicity in the blood of the subject prior to said administration, optionally about one month prior to said administration, and (b) determining a post-administration level of said marker for hepatotoxicity in the blood of the subject after said administration, optionally every week, or every 1, 2, 3, 4, 5, or 6 days.

Such methods may further comprise the step of: (c) upon detection of hepatotoxicity by biochemical or clinical signs, administering to the subject a therapeutically effective amount of an immunosuppressant (e.g., glucocorticoid) for a therapeutic treatment time period, e.g., at least about 5 to about 8 weeks (e.g., 5, 6, 7 or 8 weeks), and is preferably followed by a tapering time period during which tapering amounts of the immunosuppressant (e.g. glucocorticoid) are administered for a time period of about 2 to 4 weeks (e.g. 3 weeks). For example, the step (c) comprises, upon detection of hepatotoxicity by (i) a post-administration level of said marker of hepatotoxicity greater than the upper limit of normal (ULN), or (ii) a post-administration level of said marker of hepatotoxicity greater than or equal to twice the baseline level of said marker of hepatotoxicity, administering to the subject a therapeutically effective amount of a glucocorticoid for a time period of at least about 5 to about 8 weeks or longer (e.g., 5, 6, 7 or 8 weeks or longer), followed by tapering amounts of the glucocorticoid for a time period of about 2, 3 or 4 weeks. In any of such embodiments, the marker of hepatotoxicity is ALT and/or AST, preferably ALT. In some embodiments, upon said detection, a prednisone equivalent is administered at a prednisone-equivalent dose of 40 mg/day for a time period of about 5 weeks, followed by tapering amounts of the prednisone equivalent for a time period of about 3 weeks.

“Prophylactic” glucocorticoid or systemic immunosuppressant treatment refers to the administration of a glucocorticoid or immunosuppressant to prevent hepatotoxicity and/or to prevent an increase in measured ALT levels in the subject. “Therapeutic” glucocorticoid or immunosuppressant treatment refers to the administration of a glucocorticoid or immunosuppressant to reduce hepatotoxicity caused by administration of an AAV virus and/or to reduce an elevated ALT concentration in the bloodstream of the subject caused by administration of an AAV virus. In certain embodiments, prophylactic or therapeutic glucocorticoid treatment may comprise administration of a prednisone-equivalent dose of at least 5, 10, 15, 20, 25, 30, 35, 40, 45, 50, 55, 60, or more mg/day, e.g. a prednisone-equivalent dose of between about 10 mg/day and about 60 mg/day of the glucocorticoid to the subject. In certain embodiments, prophylactic or therapeutic glucocorticoid treatment of a subject may occur over a continuous period of at least about 3, 4, 5, 6, 7, 8, 9, 10, 11, 12, 13 weeks, or more, followed by a period of administering tapering amounts. Glucocorticoid that find use in the methods described herein include any known or routinely-employed glucocorticoid including, for example, dexamethasone, prednisone, prednisolone, fludrocortisone, hydrocortisone, budesonide and the like, at the equivalent doses for the same time periods.

Other systemic immunosuppressants that may be administered in prophylactically effective or therapeutically effective doses to prevent or reduce hepatotoxicity include (1) calcineurin inhibitors, e.g. tacrolimus or cyclosporine, (2) antiproliferative agents or IMDH inhibitors, e.g. mycophenolate, leflunomide or azathioprine, (3) mTOR inhibitors, e.g., sirolimus or everolimus. (4) janus kinase inhibitors, e.g. tofacitinib, or (5) immunosuppressant antibodies.

### Detection of Anti-AAV Antibodies

To maximize the likelihood of successful liver transduction with systemic AAV-mediated therapeutic gene transfer, prior to administration of an AAV particle in a therapeutic regimen to a human patient as described above, the prospective patient may be assessed for the presence of anti-AAV capsid antibodies or anti-AAV neutralizing antibodies that are capable of blocking cell transduction or otherwise reduce the overall efficiency of the therapeutic regimen. Such antibodies may be present in the serum of the prospective patient and may be directed against an AAV capsid of any serotype. In one embodiment, the serotype against which pre-existing antibodies are directed is AAV5.

Methods to detect pre-existing AAV immunity are well known and routinely employed in the art and include cell-based in vitro transduction inhibition (TI) assays, in vivo (e.g., in mice) TI assays, and ELISA-based detection of total anti-capsid antibodies (TAb) (see, e.g., Masat et al., Discov. Med., vol. 15, pp. 379-389 and Boutin et al., (2010) Hum. Gene Ther., vol. 21, pp. 704-712). TI assays may employ host cells into which an AAV-inducible reporter vector has been previously introduced. The reporter vector may comprise an inducible reporter gene such as GFP, etc. whose expression is induced upon transduction of the host cell by an AAV virus. Anti-AAV capsid antibodies present in human serum that are capable of preventing/reducing host cell transduction would thereby reduce overall expression of the reporter gene in the system. Therefore, such assays may be employed to detect the presence of anti-AAV capsid antibodies in human serum that are capable of preventing/reducing cell transduction by the therapeutic AAV particle.

The assays to detect anti-AAV capsid antibodies may employ solid-phase-bound AAV capsid as a “capture agent” over which human serum is passed, thereby allowing anti-capsid antibodies present in the serum to bind to the solid-phase-bound capsid “capture agent”. Once washed to remove non-specific binding, a “detection agent” may be employed to detect the presence of anti-capsid antibodies bound to the capture agent. The detection agent may be an antibody, an AAV capsid, or the like, and may be detectably-labeled to aid in detection and quantitation of bound anti-capsid antibody. In one embodiment, the detection agent is labeled with ruthenium or a ruthenium-complex that may be detected using electrochemiluminescence techniques and equipment.

The same above-described methodology may be employed to assess and detect the generation of an anti-AAV capsid immune response in a patient previously treated with a therapeutic AAV virus of interest. As such, not only may these techniques be employed to assess the presence of anti-AAV capsid antibodies prior to treatment with a therapeutic AAV virus, they may also be employed to assess and measure the induction of an immune response against the administered therapeutic AAV virus after administration. As such, contemplated herein are methods that combine techniques for detecting anti-AAV capsid antibodies in human serum and administration of a therapeutic AAV virus for the treatment of Fabry Disease, wherein the techniques for detecting anti-AAV capsid antibodies in human serum may be performed either prior to or after administration of the therapeutic AAV virus.

Other aspects and advantages of the present disclosure will be understood upon consideration of the following illustrative examples.

### Examples

### Example 1: Administration of AAV Particles to Non-Human Primates and Mice

Data from nonclinical studies with rAAV particles in mice and non-human primates (NHP) demonstrated that a single IV administration of vehicle or rAAV particles resulted in a dose-dependent increase and sustained production of circulating functional hC1-INH expression to levels expected to be therapeutic in human patients with HAE.

SerpinG1^{-/-} mice were administered vehicle or rAAV particles comprising a recombinant vector construct described herein and an AAV type capsid, at doses of 2E13 and 6E13 vector genomes per kg body weight (vg/kg). The rAAV particles were in a formulation of 10 mM Tris pH 7.4, 120 mM NaCl, 75 mM Trehalose, and 0.1% F-68 Pluronic (Poloxamer 188). Both doses were similarly effective as purified hCINH protein at normalizing vascular permeability in SerpinG1^{-/-} mice.

At 2E13 vg/kg, SerpinG1^{-/-} mice had mean functional plasma hC1INH levels that were similar to normal total hC1INH levels (160-320 μg/mL) and/or human functional plasma levels (1 IU/mL).

Doses ranging of vehicle or rAAV particles comprising a recombinant vector construct described herein and an AAV type capsid from 2E14 vg/kg up to 6E14 vg/kg were administered to cynomolgus monkeys. The vehicle and rAAV particles were in a formulation of 20 mM Tris pH 7.4, 120 mM Sodium Chloride (NaCl), 75 mM Trehalose, and 0.1% F-68 Pluronic (Poloxamer 188). Measures of safety were monitored, including weekly physicals, body weight measurements, monitoring for anti-AAV5 antibody response, coagulation parameters, and liver enzyme levels such as ALT and AST. Dosing ranging from 2E14 vg/kg to 5.1E14 vg/kg were also administered to cynomolgus monkeys and observed for 17 weeks post dosing as described above. The primates were monitored for adverse clinical signs, and all major organs assessed for pathology. There were no serious adverse effects observed over the monitoring period of 12 to 17 weeks. Minimal to moderate increases in ALT/AST were observed in 4 of 28 animals administered rAAV expressing functional C1-INH (at 2E14 or 6E14 vg/kg) and 1 of 6 animals administered the vehicle control, without clinical symptoms and with all increases resolving within 1 to 2 weeks. No thromboembolic (TE) events were observed in this study, nor was there any change in coagulation parameters. All increases in ALT/AST resolved, and no clinical symptoms were observed. These data indicate that doses up to 6E14 vg/kg are expected to be safe in humans.

In cynomolgus monkeys, plasma hC1-INH was expressed at an average concentration of greater than 20 μg/mL following rAAV particle administration at doses of 2E14 vg/kg and greater, which is a plasma concentration associated with a clinically meaningful reduction in HAE attacks. Following administration, C1-INH levels in humans are expected to be within the range observed between mouse and monkey.

Based on these data, a single administration of rAAV particles at doses disclosed herein is expected to be safe and tolerable and is expected to provide direct benefit to patients through therapeutic increases in C1INH levels, thus reducing the need for on demand and prophylactic medications and allowing for reduction or prevention of HAE attacks.

To assess the effect of steroid treatment, some cynomolgus monkeys were treated prophylactically with glucocorticoids (i.e., methylprednisolone sodium succinate) prior to the administration of rAAV encoding C1INH and post administration daily for approximately 4 weeks. Other cynomolgus monkeys were not treated prophylactically with glucocorticoids prior and post administration of rAAV encoding C1INH. The animals were subsequently dosed with 2E14 to 6E14 vg/kg of rAAV encoding C1INH via a slow intravenous bolus injection. The rAAV encoding C1INH was in a formulation of 20 mM Tris pH 7.4, 120 mM Sodium Chloride (NaCl), 75 mM Trehalose Dihydrate, and 0.1% F-68 Pluronic (Poloxamer 188). Animals were followed for 12 weeks post-dose for the expression of hC1-INH protein and general health, monitored by clinical pathology and regular clinical observations. At the end of dosing in Week 13 and approximately 9 weeks after discontinuation of steroid treatment, all animals were euthanized and necropsied. The liver was evaluated by ddPCR to determine if treatment with glucocorticoids affected vector DNA and RNA copy numbers in hepatocytes. In addition, plasma hC1-INH and limited histopathology evaluations were performed to observe whether glucocorticoids had an impact on the expression. It was noted that the median plasma hC1-INH levels were higher in animals receiving prophylactic glucocorticoid treatment. Pharmacodynamic parameters of total plasma hC1-INH protein at 2E14 vg/kg and 6E14 vg/kg were greater with prophylactic glucocorticoid treatment by approximately 5-fold with no difference in Tₘₐₓ.

### Example 2: Pharmaceutical Formulation

The rAAV C1-INH vector particles comprising an AAV5 type capsid and a recombinant vector construct described herein, e.g. any of SEQ ID NOs: 9, 20-36, 57 or 58, are provided in a liquid formulation suitable as a physiologically compatible IV solution for intravenous administration, that is stable for long periods of time, e.g. 1 or 2 years, while frozen at ≤−20° C. (at about minus 20° C. or less), ≤−40° C. (at about minus 40° C. or less), or ≤−60° C. (at about minus 60° C. or less). The liquid formulation is also stable for a time period of, e.g., at least 6 or 12 months under appropriate accelerated storage conditions.

AAV capsids in formulations with 10 to 30 mM Tris buffer at pH 7-8 have shown improved capsid stability and potency with reduced deamidation. Trehalose does not crystallize during freezing and therefore improves formulations.

Tris buffer was selected to maintain the target pH (7.4) of the solution long-term and accelerated stability testing conditions. The pH stability of the formulation was evaluated under three different storage conditions: long term (≤−60° C.), accelerated (2-8° C.), and stressed (25° C./60% RH). For all tested conditions, there were no significant changes of pH over time.

Sodium chloride within certain concentration ranges maintains capsid colloidal stability and solution clarity. In the absence of NaCl, the rAAV C1-INH vector particles may precipitate out of solution. An aqueous solution containing at least 50 mM NaCl is necessary to reduce the overall haziness of the rAAV C1-INH vector particle solution and maintain solubility of the rAAV C1-INH vector particles. Increasing NaCl concentrations from 50 to 100 mM improved stability, while NaCl concentrations from 100 mM to 165 mM showed comparable results. A concentration of 120 mM NaCl within that range was selected to maintain the stabilizing effect while maintaining an isotonic solution.

Various bulking agents that are cryopreservative or cryoprotectant agents, were tested for their ability to maintain stability of the liquid formulation under freezing temperature conditions. Comparison of sugars such as trehalose with polyols such as mannitol showed that trehalose was superior at maintaining stability. 2.5% trehalose dihydrate was determined to be the optimal amount of trehalose cryoprotectant in the presence of 120 mM NaCl, and achieved the stabilizing effect while maintaining an isotonic solution.

Surfactants reduce adsorption of rAAV C1-INH vector particles to contact surfaces, and thus reduce precipitation and increase formulation stability. While 0.2% w/v poloxamer, e.g. poloxamer 188, was previously determined to be desirable for other rAAV C1-INH vector particle formulations, the rAAV particles of the invention containing nucleic acid encoding functional C1-INH have been shown to be stable when lesser amounts are used. Varying levels of poloxamer concentrations (0, 0.05%, 0.1% and 0.2% (w/v) were analyzed with a concentration of 8E13 vg/mL rAAV5 particles. The adsorptive properties of the poloxamer were observed to mitigate adsorptive losses. As little as 0.05% poloxamer shows retention of the monomer and prevents the loss detected with absence of poloxamer. A concentration of poloxamer 188 at 0.1% w/v was suitable to maintain the stability of the liquid formulation under all tested conditions.

The formulation was able to maintain stability of a relatively high AAV particle concentration. The final aqueous formulation of rAAV C1-INH vector particles at a concentration of about 1E13 to about 1E14 vg/mL, with 20 mM Tris (20 mM or −2.4 mg/ml Tris), 120 mM or ˜7 mg/ml sodium chloride, 2.5% trehalose dihydrate, and 0.1% w/v or 1 mg/ml poloxamer 188, resulted in a final solution essentially free of particles while maintaining overall product stability at the intended use and storage conditions. Testing showed that the formulation is expected to be stable for up to 2 years at about −60° C. (minus 60) or less.

### Example 3: pH Ranging Studies—Capsid Stability, Deamidation and Potency

Storage and transport of the pharmaceutical composition at ultra-low frozen conditions (≤−60° C.) can present supply chain challenges and can reduce the stability and potency of the pharmaceutical composition. Accordingly, the pharmaceutical composition of various embodiments can be stored at higher temperatures such as −20° C. with little to no effect on stability and potency, which alleviates the difficulty of using ultra-low frozen temperatures and the resulting logistics challenges.

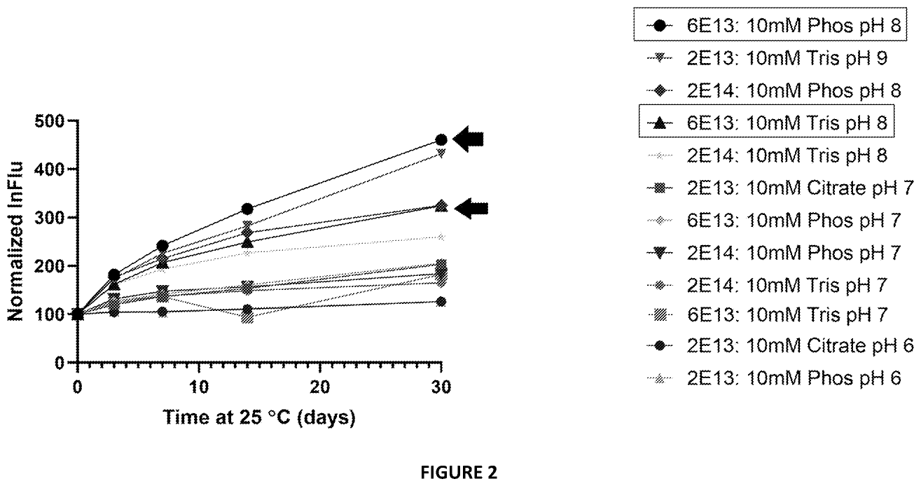

rAAV C1-INH vector capsid formulations comprising 10 mM phosphate, Tris or citrate buffer, 120 mM sodium chloride, 2.5% trehalose and 0.1% P-188 were tested for stability at pH levels from 6-9 and concentrations ranging from 2e13 vector genomes/mL to 6e13 vg/mL. The formulations were subjected to zonal ultracentrifugation (ZUC) to remove impurities prior to analysis. FIG. 2 is a graph showing a thermal based capsid integrity (TBCI) analysis of capsid stability of rAAV C1-INH vector in formulations with different buffers (Tris, Phosphate, and Citrate) and pH (6-9). The TCBI analysis is disclosed in WO 2021/062164, which is incorporated by reference in its entirety. The TCBI analysis includes the step of combining a DNA binding dye that is fluorescent when bound to DNA to rAAV C1-INH vector in solution. The solution is heated to a temperature where the capsid become unstable and the previous encapsulated vector genome is separated from the capsid and binds to the dye. Fluorescence being visualized at lower temperatures indicates that the capsids of the rAAV C1-INH vector have a lower stability. Fluorescence being visualized at higher temperatures indicates that the capsids of the rAAV C1-INH vector have a higher stability. Results from the TCBI analysis indicated that capsid stability depended strongly on buffer pH and showed no titer-dependence. Capsids in Tris buffer formulations were shown to be more stable than capsids in phosphate buffer formulations at the same pH, with the best stability observed at pH 7-8 (FIG. 2). For example as indicated by the arrows in graph and identified by the boxes in legend of FIG. 2, the 6e13 vg/ml concentration at pH 8 showed a greater than 1.5-fold increase in stability when Tris buffer is used compared to phosphate buffer.

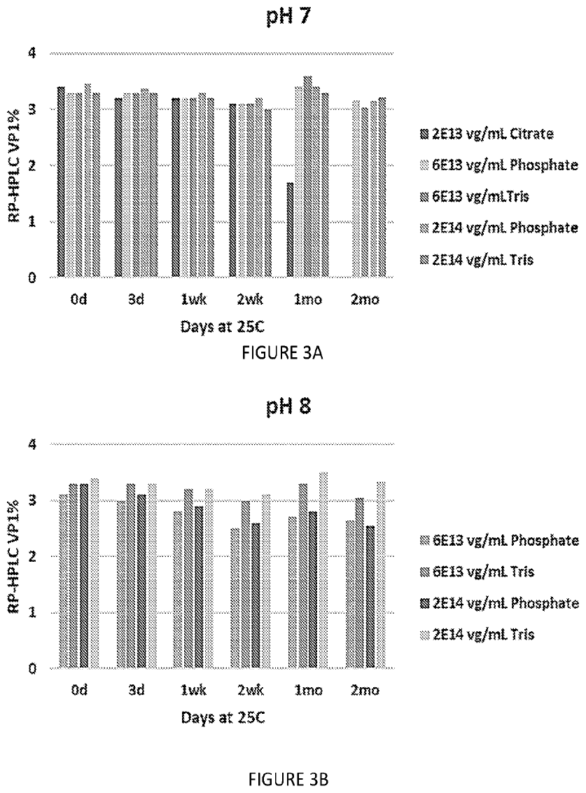

The VP content of AAV formulations (ZUC) were analyzed by reverse phase-performance liquid chromatography (RP-HPLC) after subjecting the formulations to accelerated stability testing conditions (i.e. 25° C.). Formulations comprising 2e13 vg/ml citrate, 6e13 vg/ml phosphate, 2e14 vg/ml phosphate, 6e13 vg/ml Tris or 2e14 vg/ml Tris were analyzed at pH 7, and formulations comprising 6e13 vg/ml phosphate, 2e14 vg/ml phosphate, 6e13 vg/ml Tris or 2e14 vg/ml Tris were analyzed at pH 8 were subjected to accelerated testing conditions (i.e. 25° C.). The percentage of VP1 relative to the total concentration of VP1, VP2, and VP3 proteins in the capsids was determined by RP-HPLC on day 0, day 3, 1-week, 2-weeks, 1-month and 2-months. Citrate buffer resulted in a dramatic loss of VP1. FIGS. 3A and 3B are graphs showing percentage of VP1 in the rAAV C1-INH vector in formulations with different buffers (Tris, Phosphate, and Citrate) at pH 7 and 8 after storage for 0 days, 3 days, 1 week, 2 weeks, 1 month, and 2 months. The data collected indicates VP1% for both phosphate and Tris were similar at pH 7 and 8 regardless of concentration. Further, at pH 8 VP1% was higher for both 6e13 and 2e14 vg/ml of Tris buffer when compared to phosphate buffer at its corresponding concentration (FIGS. 3A and 3B).

VP1 deamidation was analyzed by liquid chromatography-mass spectrometry. AAV capsids formulations with 10 mM phosphate, Tris or citrate buffer, 120 mM sodium chloride, 2.5% trehalose and 0.1% P-188 were tested for percentage of deamidation. The percentage of VP1 deamidation of formulations comprising phosphate or Tris at 2e14 vg/ml and pH 7 or pH 8 were evaluated under accelerated testing conditions (i.e. 25° C.) at day 0, 2-weeks and 1-month. Results indicate no significant deamidation at pH 7 under accelerated conditions for any of the buffers. Deamidation was shown to be pH-dependent with % deamidation increasing at a faster rate over time for formulations at higher pH.

Potency was determined by reverse transcriptase digital droplet polymerase chain reaction (RT-dd-PCR) analysis of mRNA transgene expression. AAV capsid formulations with 10 mM phosphate, Tris or citrate buffer, 120 mM sodium chloride, 2.5% trehalose and 0.1% P-188 were tested for percentage of potency (i.e. mRNA expression) relative to reference. The % potency of formulations comprising phosphate or Tris at 2e14 vg/ml and pH 7 or pH 8, or Tris at 2e13 vg/ml and pH 9 were evaluated under accelerated testing conditions (i.e. 25° C.) at day 0, 2-weeks and 1-month. A pH of 8 provided the best potency for Tris buffer.

### Example 4: Formulation Stability at pH 7.4

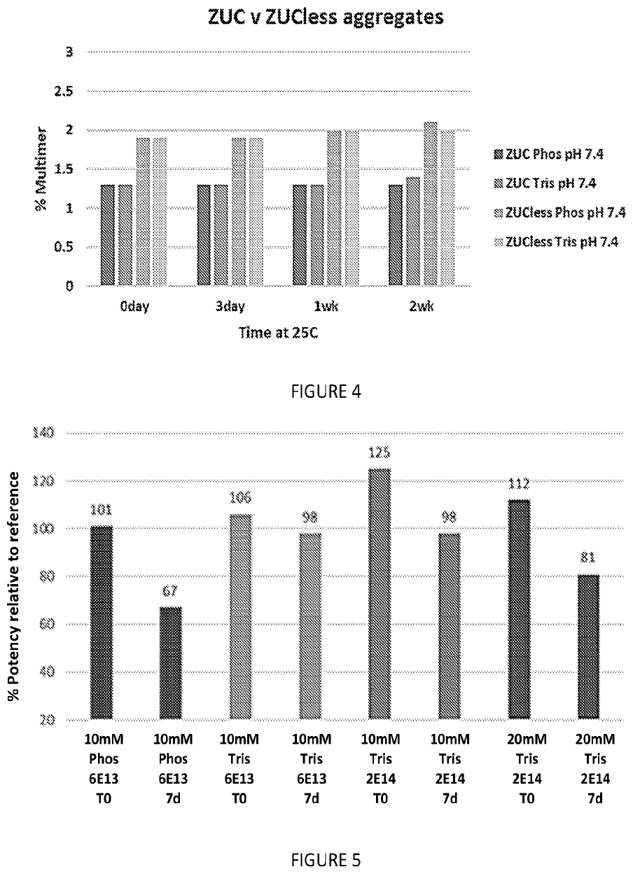

AAV capsid formulations at pH 7.4 comprising phosphate or Tris buffer, 120 mM sodium chloride, 2.5% trehalose and 0.1% P-188 were prepared and evaluated to determine the effect of removing impurities by zonal centrifugation (ZUC) or not (ZUCless) on aggregation of vector particles. Formulations comprising 10 mM phosphate at 6e13 vg/ml, 10 mM Tris at 6e13 vg/ml, 10 mM Tris at 2e14 vg/ml and 20 mM Tris at 2e14 vg/ml were purified by ZUC and compared to unpurified formulations comprising 10 mM phosphate at 6e13 vg/ml and 10 mM Tris at 6e13 vg/ml. Aggregation was determined by % multimer measured by size exclusion chromatography, with elution of molecules monitored by UV absorbance (SEC-UV). ZUCless formulated bulk drug substances (FBDS) showed a −0.5% higher amount of multimers (1.5-fold higher) when compared to ZUC FBDS. FIG. 4 is a graph showing the percentage of rAAV C1-INH vector multimers (i.e., aggregation) in formulations with different buffers (Tris, Phosphate, and Citrate) at pH 7.4. The aggregation for ZUCless formulations had similar % multimer for both phosphate and Tris (FIG. 4).

Potency was determined by RT-dd-PCR analysis of mRNA transgene expression. Formulations with a vector concentration of 6e13 vg/ml and phosphate or Tris buffer at pH 7.4 were compared. The % potency relative to reference was measured at day 0, 1 month at 60° C., 1 month at 2-8° C. and 1 month at 25° C.

Frozen excursion tests were performed on ZUC formulations at −40° C. to determine potency of AAV formulations. Formulations comprising 10-20 mM Tris or phosphate at concentrations of 6e13 or 2e14 were analyzed for potency on day 0 and day 7. No significant deamidation was observed for all formulations. FIG. 5 is a graph showing potency (percentage relative to a reference sample) of different rAAV C1-INH vector concentrations (6×10e13 vector genomes (vg)/milliliter (mL) and 2×10e14 vg/mL) in formulations with different buffer concentrations (10 mM Tris, 20 mM Tris, 10 mM Phosphate, and 20 mM Phosphate) after storage for 0 days and 7 days. Formulation with Tris at pH 7.4 were shown to mitigate the potency loss observed with phosphate buffer during thermal excursions under frozen conditions (FIG. 5), a phenomenon observed with cold chain logistics challenges.

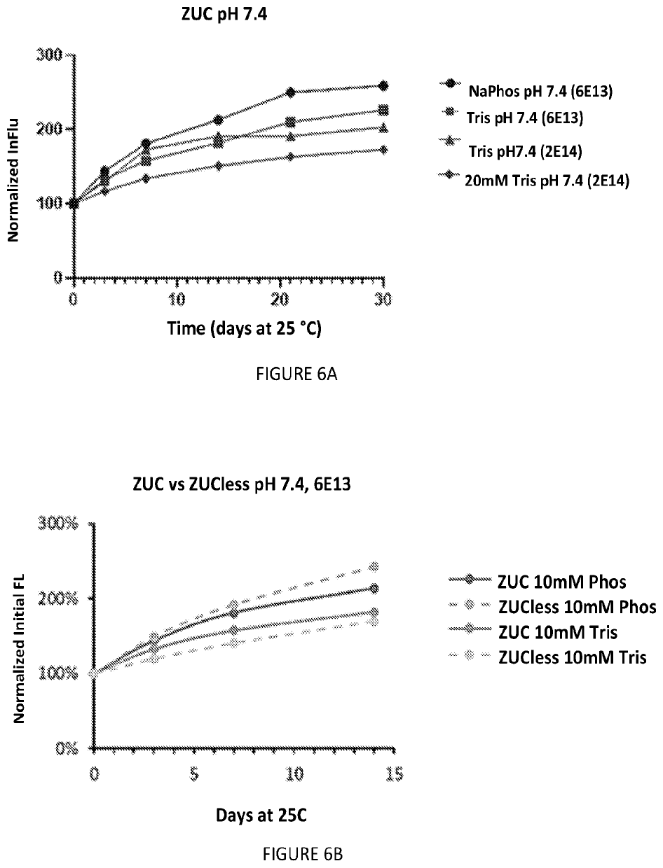

Capsid stability for ZUC vs. ZUCless formulations were tested for AAV formulations comprising phosphate or Tris buffer, 120 mM sodium chloride, 2.5% trehalose and 0.1% P-188 at pH 7.4. Formulations were at concentrations of 6e13 or 2e14 vg/ml and comprised 10 mM phosphate, 10 mM Tris or 20 mM Tris. Formulations were subjected to accelerated testing conditions at 25° C. and data was collected over 30 days. FIGS. 6A and 6B are graphs showing a TBCI analysis of capsid stability of different rAAV C1-INH vector concentrations (6×10e13 vg/mL and 2×10e14 vg/mL) that have been purified with or without zonal ultracentrifugation (ZUC) in formulations with different buffer concentrations (10 mM Tris, 20 mM Tris, 10 mM Phosphate, and 20 mM Phosphate) after storage at about 25° C. for 0 days to 30 days. Results indicated ZUC and ZUCless Tris formulations had greater stability when compared to phosphate formulations, with ZUCless Tris formulations being more stable than ZUC Tris formulations, and 20 mM Tris ZUC formulations was determined to have the best stability (FIGS. 6A and 6B). Overall, 20 mM Tris pH 7.4 formulations presented the best capsid stability.

### Example 5: Analysis of Formulation Stability and Potency after Extended Storage Time Periods

AAV capsid formulations at pH 7.4 comprising 20 mM Tris buffer, 120 mM sodium chloride, 2.5% trehalose and 0.1% P-188 were prepared and evaluated to stability and potency of the AAV capsid formulations after storage for pre-determined time periods.

The AAV capsid formulations were stored at −20° C. or −40° C. for 1 week, 2 weeks, 1 month, 3 months, 6 months, 9 months, 12 months, 18 months, or 24 months. The stability of the samples stored at −20° C. or −40° C. for up to 24 months were confirmed by monitoring the potency of the samples. To assess potency, human hepatic cells (HepG2 cells) were transduced with AAV capsid formulations at different multiplicities of infection (MOI). After a pre-determined time period, the level of C1-1NH expression by the transduced cells was quantified using bio-layer interferometry of the cell culture media that was cultured with the transduced cells and contains C1-1NH. The C1-1NH concentration is identified from comparisons with standard C1-1NH control concentration using log-log plots and a constrained parallel line fit.

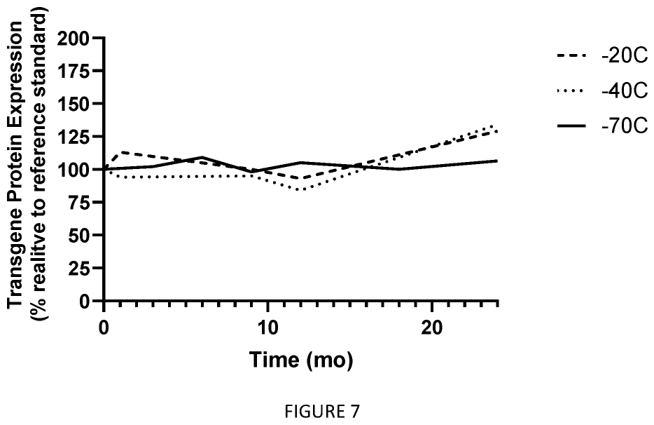

FIG. 7 is a graph showing the potency of rAAV C1-INH vector in a formulation at pH 7.4 comprising 20 mM Tris buffer, 120 mM sodium chloride, 2.5% trehalose and 0.1% P-188 when stored at −20° C., −40° C., and −70° C. for 0 months to 24 months. As shown in FIG. 7, AAV capsid formulations when stored at −20° C. or −40° C. for up to 24 months showed comparable or higher potency as compared storage at −70° C. for up to 24 months when the capsid formulations were used to transduce HepG2 cells. In this case, potency is understood to mean the infectiousness of the rAAV C1-INH vector in the capsid formulations and is assessed by measuring C1-1NH protein expression as described above. The potency of the rAAV C1-INH vector in capsids formulations stored at −20° C. or −40° C. for extended time periods appeared to have comparable potency (˜85% to −135%) to rAAV C1-INH vector in capsids formulations stored at −70° C. for extended time periods (˜98% to −110%). It is further noted from FIG. 7 that the rAAV C1-INH vector in the AAV capsid formulations when stored −20° C. or −40° C. for at least 16 or 17 months appeared to have greater potency than rAAV C1-INH vector in the AAV capsid formulations. For example at 24 months, the potency of the rAAV C1-INH vector in the AAV capsid formulations when stored −20° C. or −40° C. (˜129% and −134%) had substantially greater potency than rAAV C1-INH vector in the AAV capsid formulations when stored −70° C. (˜106%). This is an unexpected and significant improvement in the stability and potency of other AAV capsid formulation using different reagents. The improvement also opens up the potential for enhancing transportation logistics and reducing transportation cost while maintaining or enhancing the efficacy of the AAV capsid formulations.

### Example 6: Administration of AAV Particles to Human Subjects

A clinical study is performed on subjects with frequent HAE attacks who are currently using either on-demand therapy or routine long-term prophylaxis HAE medication. The objective is to demonstrate a clinically meaningful increase in plasma levels of functional C1-INH (C1-INH(f)) in subjects with HAE after a single intravenous administration of the rAAV particles. A clinically meaningful increase in C1-INH(f) levels (defined as an increase in C1 INH[f] of ≥10%, which corresponds to an approximate increase in antigenic C1 INH levels of approximately 20 μg/mL) will be demonstrated. Longhurst et al., N Engl J Med., 376(12):1131 40 (2017).

Human subjects are administered rAAV particles comprising an AAV5 type capsid and a recombinant AAV vector construct described herein, at one of five dose levels to assess the efficacy, safety and tolerability of the rAAV particles at the indicated doses.

Subjects with baseline functional C1-INH (C1-INH(f)) level below 50% of the lower limit of normal (LLN) are administered the rAAV particles at the desired dose, in a single intravenous infusion, and are followed for 5 years to evaluate durability of the response. A proportion of subjects in at least one dose cohort will achieve a clinically significant increase in expression of C1-INH(f), for example, as measured by an increase in plasma C1 INH[f] of ≥10%, which corresponds to an approximate increase in antigenic C1 INH levels of approximately 20 μg/mL, by Week 8, 24 or 48 post-infusion. A durable response will last at least 6 months, 1 year, 1.5 years, 2 years, 3 years, 4 years or 5 years or longer. It is noted from prior modelling work with C1-inhibitor that an approximate 50% attack risk reduction was identified with an increase of 7.7% functional (absolute) or −20 μg/mL in C1-INH.

A proportion of subjects in at least one dose cohort will achieve any one or more of (a) a reduction in the number and/or severity of HAE attacks, optionally a reduction in (i) moderate and severe HAE attacks and/or (ii) high morbidity HAE attacks; (b) a reduction in the use of HAE-specific medication (on-demand therapy and/or prophylactic therapy); and/or (c) an improvement in health-related quality of life, as measured, for example, by any one of (i) Angioedema Control Test (AECT), (ii) Angioedema Quality of Life (AE QoL), (iii) Treatment Satisfaction Questionnaire for Medication (TSQM 9), or (iv) EuroQoL 5 dimension 5 level (EQ 5D 5L). For example, the improvements may be achieved by Week 24 or six months, Week 48 or one year, and may be maintained for at least 1, 2, 3, 4 or 5 years or longer.

Moreover, the administration of the rAAV particles will be demonstrated to be safe for most patients, e.g., low incidence of treatment-emergent serious adverse events, low incidence of complement activation or hypersensitivity, low incidence of abnormalities in coagulation markers, and either low incidence of hepatic transaminase elevation or transient elevations that resolve after glucocorticoid therapy.

### Administration

The rAAV particles are administered by intravenous (IV) infusion. Just prior to the infusion, subjects receive a prophylactic short term IV injection of 1000 IU plasma derived C1 INH (independent from previous HAE long term prophylaxis treatment), as a precautionary safety measure to ensure normal levels of C1 INH during infusion. Subjects also begin a prophylactic glucocorticosteroid regimen before infusion, on the day of infusion.

Systemic immunosuppressive agents other than the glucocorticosteroid regimen are avoided, starting 30 days before the start of screening through the end of the study. Alternative, non steroidal systemic immunosuppressive agents are considered for use on a case by case basis, e.g., if glucocorticoid use is deemed clinically ineffective, not tolerated, and/or contraindicated).

The following medications should generally be avoided in the HAE patient population and are also avoided during the study: angiotensin converting enzyme inhibitors; estrogen containing medications with systemic absorption (such as oral contraceptives or hormonal replacement therapy).

Patients should abstain from alcohol ingestion, herbal and natural remedies, dietary supplements, and hepatotoxic medications from screening until conclusion of the study.

The following medications or vaccines should be avoided during or in proximity of the start/termination of oral glucocorticosteroid therapy: vaccines; hepatotoxic therapies; non steroidal anti inflammatory drugs (NSAIDs) should be avoided for at least 14 days prior to Day 1 and through the duration of glucocorticosteroid therapy. The use of cyclooxygenase (COX) 2 inhibitors is permitted but should be limited during period of glucocorticosteroid therapy. Acetaminophen, at doses of ≤2 grams/day, is permitted for use any time during the study; however, use should be minimized where possible.

Subjects continue to use their HAE medication for on demand treatment of HAE attacks as prescribed by their treating physician prior to enrollment into the study. Any self administered medications approved for on demand use are appropriate for use during the study. HAE rescue medications such as C1 INH concentrate, recombinant human C1 INH or icatibant may be used.

### Inclusion and Exclusion Criteria

The clinical study inclusion criteria include the following: (1) age 18 years or older, (2) diagnosis of Type I or Type II hereditary angioedema (HAE) due to (i) C1-INH deficiency with functional or immunogenic C1-INH levels below 50% of the lower limit of normal (LLN) as assessed by a central laboratory assessment and (ii) confirmed by genotyping of the C1-INH gene (SERPING1), and (iii) C4 levels below laboratory reference range prior to the administration of rAAV particles; (3) currently using an HAE medication regimen that consists of a a) a routine long-term prophylactic treatment regimen with C1-INH replacement therapy (dose expansion study only), lanadelumab (an anti-kallikrein antibody) or berotralstat (a kallikrein inhibitor), for at least 6 months prior to administration based on historical attack frequency of at least 1 attack per month (on average) prior to initiation of routine prophylactic treatment, or b) an on-demand therapy regimen for the last 6 months for attack frequency of at least 1 attack per month (on average), and (4) have a body mass index ≤35 kg/m2 or body weight of ≤100 kg. Additional considerations included compliance with the HAE medication regimen, training in self-administering acute attack treatment and able to adequately manage acute attacks, willingness to abstain from alcohol, herbal and natural remedies, dietary supplements, and hepatotoxic medications from screening through at least 52 weeks post-administration of rAAV particle, and willingness to follow specified guidance for donation of semen, oocyte, blood, organs or tissue.

The clinical study exclusion criteria include the following: (1) evidence of an active infection or chronic infection, including severe acute respiratory syndrome coronavirus 2 (SARS-CoV-2), or immunosuppressive disorder; (2) contraindication to glucocorticoid (GCS), including a diagnosis of glaucoma or untreated osteoporosis; (3) active malignancy (except non-melanoma skin cancer) autoimmune, metabolic (i.e. diabetes), hematologic, cardiac, or renal disease that is of clinical significance defined as requiring regular medical attention and treatment, (4) a history of clinically relevant liver disease of any etiology (e.g., nonalcoholic steatohepatitis [NASH], or chronic viral hepatitis B or C [HBV or HCV], or autoimmune hepatitis) as assessed by liver ultrasound and/or other diagnostic test, (5) any disease or condition which would prevent patient from fully complying with the requirements of study, expose them to unacceptable risk when following the study procedures, and/or require continuous medical attention which may interfere with interpretation of study data, including possible glucocorticoid treatment, (6) a history of clinically significant venous thrombosis or arterial thrombosis, indwelling vascular access catheter, known underlying risk for thrombosis (i.e. inherited thrombophilia-like factor V Leiden) or currently using chronic anticoagulation, (7) having a substance use disorder and/or active major depressive disorder in the past 12 months prior to screening, or psychiatric disorder that would interfere with completion of the study, (8) known allergy or hypersensitivity to rAAV C1-INH, (9) taking any prohibited medications, (10) having prior gene therapy treatment, (11) having any long-term use (i.e. 1 year of cumulative use) of attenuated androgens in the last 4 years and/or having used any attenuated androgens in the last year,

Further the following diagnostic clinical study exclusion criteria apply: (1) detectable antibodies to AAV5 capsid (i.e. seropositivity); (2) clinically abnormal laboratory values indicative of a concomitant metabolic, renal or hematology disease including serum creatine greater than or equal to 1.5 mg/dL, hemoglobin A1c greater than or equal to 8.0%, glucose greater than or equal to 250 mg/dL, or significant thrombocytopenia (i.e. platelet count greater than 100×10⁹/L; (3) liver dysfunction with any one of the following abnormal laboratory results: (a) elevation in any of ALT (alanine transaminase), AST (aspartate aminotransferase), GGT (gamma-glutamyltransferase), bilirubin, or alkaline phosphatase to more than 1.25 times the upper limit of normal (ULN), or the international normalized ration (INR) being greater than or equal to 1.2; (b) significant liver fibrosis (Grade 3 or higher) as diagnosed by transient elastography (FibroScan), (c) a liver biopsy of Grade 3 or higher as rated on a scale of 0-4 or positive on the Batts Ludwig or METAVIR (Meta analysis of Histological Data in Viral Hepatitis) scoring systems or equivalent grade of fibrosis on an alternative scale, (d) a positive serologic enhanced liver fibrosis (ELF) marker test with a score of greater than or equal to 7.2; or (e) chronic or active infection with hepatitis B or C.

### Monitoring of Safety and Efficacy

Prior to infusion of the rAAV particles, subjects are evaluated for: (1) baseline physical examination; (2) baseline clinical laboratory tests, including antigenic and functional levels of (a) C1-INH, serum C4, high molecular weight kininogen (HMWK, also known as Fitzgerald factor) and C1q, and (b) liver enzyme tests, including ALT, AST, GGT, LDH, total bilirubin and alkaline phosphatase; (c) baseline AAV5 antibody detection; (3) measures of health-related quality of life (HRQoL), e.g. Angioedema Quality of Life Questionnaire (AE-QOL) score, Angioedema Control Test (AECT) score, Treatment Satisfaction Questionnaire for Medication (TSQM-9) score, EuroQoL-5D-5L (EQ-5D-5L) score, and Patient Global Impression of Severity (PSI-S) score and/or Patient Global Impression of Change (PGI C) score; (4) baseline levels of other parameters monitored during the study; and (5) SERPING1 genotyping, if permitted.

After infusion of the rAAV particles, HAE biomarker parameters that are monitored include: (1) weekly plasma or serum C1-INH functional levels (C1-INH(f)) until week 12 and biweekly weeks 13-24; (2) biweekly C4 complement; (3) HMWK (intact and cleaved); (4) optionally C1q levels; and (5) plasma levels of C1 INH activity on C1s by ELISA, C1 INH protein by nephelometry, and C1 INH protein by LCMS MS. An improvement in any one or more of the HAE biomarker parameters is observed.

The following additional indicators of efficacy are monitored: (1) time-normalized number of HAE attacks, (2) number of moderate and severe HAE attacks, (3) number of high morbidity HAE attacks; (4) use of any HAE specific medication (on demand therapy, and prophylactic therapy); and (5) change from baseline in health-related quality of life (HRQoL), e.g. as measured by AE-QoL, AECT, EQ-5D-5L, PGI-S, PGI-C and TSQM-9 score. A clinically significant improvement in any one or more of these parameters is observed.

The incidence of adverse events and serious adverse events are monitored. Subjects are monitored for clinical laboratory test results (serum chemistry, hematology, coagulation parameters), vital signs, physical examination findings, vector shedding (in blood, urine, semen, stool, saliva) and liver test results (including ALT, AST, GGT, LDH, total bilirubin, and alkaline phosphatase). Other parameters monitored include detection of antibodies against AAV5 capsid and C1 INH; assessment of C1 INH and capsid specific cellular immunity, e.g. by ELISpot assay on PBMC; evaluation of complement activation after treatment (C3, C3a, Bb, sC5b 9 levels).

Each subject will have comprehensive and ongoing monitoring of safety and liver function throughout the study. Additionally, a liver ultrasound will be performed to monitor for any clinically relevant liver disease. Additional monitoring of liver function may be performed.

The administration of the rAAV particles is safe, e.g., no clinically significant treatment-emergent serious adverse events, and no clinically significant changes in standard clinical laboratory values or markers of hepatotoxicity such as AST and/or ALT (or if changes occur, most are transient or resolve after treatment with systemic immunosuppressant). Immune response against the AAV capsid is limited, as is blood biodistribution, and urine, stool, semen, and saliva vector shedding.

### Prophylactic Glucocorticoids (GCS) Therapy

Liver inflammation may be reduced or avoided by prophylactic glucocorticoid therapy. A 16-week prophylactic glucocorticoids course is administered with a prednisone-equivalent starting dose of 40 mg/day, beginning on Day 1 pre-infusion, for a time period of 13 weeks dosing at 40 mg/day, followed by a 3-week dose taper beginning at Week 14 (to a prednisone-equivalent dose of 30 mg/day for a week, 20 mg/day for a week, and 10 mg/day for a week). On the day of infusion, prophylactic glucocorticoids are administered at a minimum 3 hours before rAAV particle infusion. ALT and AST levels are monitored weekly. If there is ALT elevation to greater than upper limit of normal (ULN) or greater than 2× baseline ALT value, during the first 12 weeks, adjustments to glucocorticoid dosing are based on clinical judgment, and liver enzymes may be monitored more frequently. Subjects should be assessed to determine whether they require vitamin D and calcium supplementation concomitant with glucocorticoid immunosuppression and whether the use of bone sparing and bone anabolic treatment are indicated before initiating the glucocorticoid administration.

### Reactive Glucocorticoid Therapy for Transient Hepatic Enzyme Elevations

Reactive glucocorticoid therapy may be initiated after the prophylactic regimen is completed, in response to mild ALT elevations that meet pre-specified criteria, or based on clinical judgment. It may be initiated if ALT is greater than the ULN or greater than 2× baseline in two consecutive assessments within 72 hours, or 3×ULN in two consecutive assessments within 48 hours. The recommended reactive CS regimen has a total duration of 8 weeks with 5 weeks of 40 mg/day prednisone-equivalent dosing, followed by a 3-week dose taper if ALT is both less than or equal to ULN and less than or equal to 2× baseline value. Liver enzymes are monitored weekly over 4 weeks in the period following discontinuation of reactive glucocorticoid therapy, or more frequently if ALT values are above the ULN.

Reactive glucocorticoids are not administered if elevations in ALT are clearly not related to the therapeutic intervention with rAAV particles (e.g., elevated ALT with concurrent increase in creatine phosphokinase (CPK) due to intensive exercise, or viral hepatitis).

The embodiments described herein are intended to be merely exemplary, and those skilled in the art will recognize, or will be able to ascertain using no more than routine experimentation, numerous equivalents of specific compounds, materials, and procedures. All such equivalents are considered to be within the scope of the disclosure.

All of the patents, patent applications and publications referred to herein are incorporated by reference herein in their entireties. Citation or identification of any reference in this application is not an admission that such reference is available as prior art to this application. The full scope of the disclosure is better understood with reference to the appended claims.

1. A method of treating a human subject with hereditary angioedema (HAE), comprising administering to the subject a single dose ranging from about 2E13 vg/kg to about 6E14 vg/kg of recombinant adeno-associated virus (rAAV) particles comprising (a) an AAV capsid with liver tropism, and (b) a recombinant vector construct comprising a nucleic acid encoding a functional C1 esterase inhibitor (C1-INH) protein operatively linked to a heterologous liver-specific transcription regulatory region.

2. The method of claim 1 wherein the dose is about 2E13 vg/kg.

3. The method of claim 1 wherein the dose is about 6E13 vg/kg.

4. The method of claim 1 wherein the dose is about 2E14 vg/kg.

5. The method of claim 1 wherein the dose is about 4E14 vg/kg.

6. The method of claim 1 wherein the dose is about 6E14 vg/kg.

7. The method of any of claims 1 to 6 wherein the functional C1-INH protein comprises an amino acid sequence at least 95%, 98% or 99% identical to amino acids 23 through 500 of SEQ ID NO: 2.

8. The method of any of claims 1-7 wherein the nucleic acid encoding the functional C1-INH comprises a nucleotide sequence at least 90%, 95%, 98% or 99% identical to SEQ ID NO: 1.

9. The method of any of claims 1-8 wherein the liver-specific transcription regulatory region comprises a fragment of an hAAT promoter and/or a fragment of an HCR enhancer/ApoE enhancer.

10. The method of any of claims 1-9 wherein the liver-specific transcription regulatory region comprises a nucleotide sequence at least 90%, 95%, 98% or 99% identical to SEQ ID NO: 3 or SEQ ID NO: 15.

11. The method of any of claims 1-10 wherein the liver-specific transcription regulatory region further comprises a nucleotide sequence at least 90%, 95%, 98% or 99% identical to SEQ ID NO: 4.

12. The method of any of claims 1-11 wherein the liver-specific transcription regulatory region comprises a nucleotide sequence at least 90%, 95%, 98% or 99% identical to SEQ ID NO: 5.

13. The method of any of claims 1-12 wherein the recombinant vector construct comprises an intron.

14. The method of claim 13 wherein the intron comprises a nucleotide sequence at least 90%, 95%, 98% or 99% identical to SEQ ID NO: 64.

15. The method of claim 13 wherein the intron comprises a nucleotide sequence at least 90%, 95%, 98% or 99% identical to SEQ ID NO: 6.

16. The method of any of claims 1-15 wherein the recombinant vector construct further comprises a polyadenylation signal.

17. The method of any of claims 1-16 wherein the subject is administered a population of rAAV particles produced by a method comprising (a) providing an insect cell comprising one or more nucleic acid constructs comprising: (i) a recombinant vector construct comprising (1) a 5′ AAV ITR and a 3′ AAV ITR, (2) a heterologous liver-specific transcription regulatory region comprising a nucleotide sequence at least 90% identical to SEQ ID NO: 4 and a nucleotide sequence at least 90% identical to SEQ ID NO: 3, (3) a nucleic acid encoding a functional C1-INH comprising an amino acid sequence at least 95% identical to amino acids 23 through 500 of SEQ ID NO: 2, and (4) a polyadenylation signal; (ii) a nucleotide sequence encoding one or more AAV Rep proteins which is operably linked to a promoter that is capable of driving expression of the Rep protein(s) in the cell; and (iii) a nucleotide sequence encoding one or more AAV5 type capsid protein which is operably linked to a promoter that is capable of driving expression of the capsid protein(s) in the cell; (b) culturing the cell under conditions permitting expression of the Rep and the capsid proteins and production of an AAV particle; and (c) recovering the AAV particle.

18. The method of claim 17 wherein the population is enriched for rAAV particles comprising full length or nearly full length vector genomes by steps that reduce the number of empty capsids.

19. The method of any of claims 1-18 wherein the recombinant vector construct comprises a nucleotide sequence at least 90%, 95%, 98% or 99% identical to any one of SEQ ID NOs: 9, 20-36, 57 or 58.

20. The method of any of claims 1-19 wherein the AAV capsid comprises an amino acid sequence at least 85%, 90% or 95% identical to any one of SEQ ID NOs: 35-51.

21. The method of any of claims 1-20 wherein the AAV capsid with liver tropism is an AAV5 type capsid, optionally at least 85%, 90% or 95% identical to SEQ ID NO: 44.

22. The method of any of the preceding claims wherein the rAAV particle is administered by intravenous infusion.

23. The method of claim 22, further comprising concurrently administering 1000 IU of C1-INH.

24. The method of any of the preceding claims wherein the subject has Type I or Type II hereditary angioedema.

25. The method of any of the preceding claims wherein the subject has a functional C1-INH level about 50% of the lower limit of normal (LLN) or lower, prior to rAAV particle administration.

26. The method of any of the preceding claims wherein the subject has a C4 complement level below normal range, prior to rAAV particle administration.

27. The method of any of the preceding claims wherein the subject is 18 or more years old.

28. The method of any of the preceding claims wherein the subject is 12 to 18 years old.

29. The method of any of the preceding claims wherein the subject has suffered HAE attacks at a frequency of at least 1 attack per month on average for at least 6 months.

30. The method of any of the preceding claims wherein the subject has mild or low burden HAE.

31. The method of any of the preceding claims wherein the subject has moderate HAE.

32. The method of any of the preceding claims wherein the subject has severe HAE.

33. The method of any of the preceding claims wherein the number of acute HAE attacks is reduced by about 5%, 10%, 15%, 20%, 25%, 30%, 35%, 40%, 45%, 50%, 55%, 60%, 65%, 70%, 75%, 80%, 85%, 90%, 95% or 100%.

34. The method of any of the preceding claims wherein the number of moderate to severe HAE attacks is reduced by about 5%, 10%, 15%, 20%, 25%, 30%, 35%, 40%, 45%, 50%, 55%, 60%, 65%, 70%, 75%, 80%, 85%, 90%, 95% or 100%.

35. The method of any of the preceding claims wherein the number of severe HAE attacks is reduced by about 5%, 10%, 15%, 20%, 25%, 30%, 35%, 40%, 45%, 50%, 55%, 60%, 65%, 70%, 75%, 80%, 85%, 90%, 95% or 100%.

36. The method of any of the preceding claims wherein the subject has received long-term prophylactic C1-INH replacement therapy, lanadelumab or berotralstat for at least 6 months prior to rAAV particle administration.

37. The method of any of the preceding claims wherein the subject does not have detectable anti-AAV5 capsid antibody in blood prior to rAAV particle administration.

38. The method of any of the preceding claims wherein the subject does not have clinically significant liver disease prior to rAAV particle administration.

39. The method of any of the preceding claims wherein the subject has ALT and/or AST levels within normal range prior to rAAV particle administration.

40. The method of any of the preceding claims, wherein the dose is effective to increase the plasma level of functional C1-INH in the subject by at least 10%, optionally to at least 70% to 130% of the lower limit of normal, optionally to 150% or less than lower limit of normal.

41. The method of any of the preceding claims, wherein the dose is effective to increase the plasma level of functional C1 INH in the subject by at least about 20 μg/mL, optionally to at least about 160 μg/mL.

42. The method of any of the preceding claims, wherein the dose is effective to increase the plasma level of functional C1 INH in the subject to a range of at least about 160 μg/mL to about 320 μg/mL.

43. The method of claims 40-42, wherein the dose maintains the increased plasma level for a period of at least about six months.

44. The method of claims 40-42, wherein the dose maintains the increased plasma level for a period of at least about one year.

45. The method of any of the preceding claims, wherein the dose is effective to reduce the number or severity of acute HAE attacks of the subject over a period of at least about six months.

46. The method of any of the preceding claims, wherein the dose is effective to reduce the number of moderate and severe acute HAE attacks of the subject over a period of at least about six months, optionally by at least about 5%, 10%, 15%, 20%, 25%, 30%, 35%, 40%, 45%, 50%, 55%, 60%, 65%, 70%, 75%, 80%, 85%, 90%, 95% or 100%.

47. The method of any of the preceding claims, wherein the dose is effective to reduce the number of high morbidity acute HAE attacks of the subject over a period of at least about six months, optionally by at least about 5%, 10%, 15%, 20%, 25%, 30%, 35%, 40%, 45%, 50%, 55%, 60%, 65%, 70%, 75%, 80%, 85%, 90%, 95% or 100%.

48. The method of any of claims 45-47 wherein the reduction in HAE attacks is maintained for at least about one year.

49. The method of any of the preceding claims, wherein the dose is effective to reduce the dose of or frequency of administration of HAE-specific therapy to the subject for acute HAE attacks, over a period of at least about six months, optionally by at least about 5%, 10%, 15%, 20%, 25%, 30%, 35%, 40%, 45%, 50%, 55%, 60%, 65%, 70%, 75%, 80%, 85%, 90%, 95% or 100%.

50. The method of claim 49 wherein the HAE-specific therapy is plasma derived C1 INH, recombinant C1 INH, bradykinin B2 receptor antagonist, plasma kallikrein inhibitor, or anti-kallikrein antibody, optionally BERINERT, RUCONEST, FIRAZYR (icatibant) or KALBITOR (ecallantide).

51. The method of any of the preceding claims, wherein the dose is effective to reduce the dose of or frequency of administration of HAE-specific prophylactic therapy to the subject, over a period of at least about six months, optionally by at least about 5%, 10%, 15%, 20%, 25%, 30%, 35%, 40%, 45%, 50%, 55%, 60%, 65%, 70%, 75%, 80%, 85%, 90%, 95% or 100%.

52. The method of claim 49 wherein the HAE-specific prophylactic therapy is plasma-derived C1-INH, recombinant C1-INH, plasma kallikrein inhibitor, anti-kallikrein antibody, or androgen, optionally CINRYZE, HAEGARDA, ORLADEYO (berotralstat), TAKHZYRO (lanadelumab), danazol, oxandrolone, or stanozolol.

53. The method of any of claims 49-52 wherein the reduction is maintained for at least about one year.

54. The method of any of the preceding claims wherein the dose is effective to improve health-related quality of life, optionally as measured by any one or more of Angioedema Quality of Life Questionnaire (AE-QOL) score, Angioedema Control Test (AECT) score, Treatment Satisfaction Questionnaire for Medication (TSQM-9), EuroQoL-5D-5L (EQ-5D-5L) score, or Patient Global Impression of Severity (PSI-S) score, over a period of at least about six months, optionally by at least about 5%, 10%, 15%, 20%, 25%, 30%, 35%, 40%, 45%, 50%, 55%, 60%, 65%, 70%, 75%, 80%, 85%, 90%, 95% or 100%.

55. The method of claim 54 wherein the improved health-related quality of life is maintained for at least about one year.

56. The method of any of the preceding claims further comprising administering to the subject a prophylactic immunosuppressant.

57. The method of claim 54 wherein the prophylactic immunosuppressant is a glucocorticoid, optionally dexamethasone, prednisone, prednisolone, fludrocortisone, hydrocortisone, or budesonide.

58. The method of claim 54 wherein the prophylactic immunosuppressant is a glucocorticoid and the prophylactically effective amount is a prednisone-equivalent dose of from 10 mg/day to 40 mg/day, optionally for a time period of at least about 13 weeks, followed by tapering amounts of the glucocorticoid for a time period of about 3 weeks.

59. The method of any of the preceding claims further comprising the step of (a) determining a baseline level of a marker of hepatotoxicity in the blood of the subject prior to rAAV particle administration, optionally about one month prior to said administration, and (b) subsequently determining a post-administration level of said marker for hepatotoxicity in the blood of the subject every week for at least 12 weeks.

60. The method of claim 59 further comprising administering to the subject a therapeutic immunosuppressant.

61. The method of claim 60 wherein the therapeutic immunosuppressant administration comprises the step of: (c) upon detection of hepatotoxicity by biochemical or clinical signs, administering to the subject a therapeutically effective amount of a systemic immunosuppressant to reduce hepatotoxicity.

62. The method of claim 61 wherein detection of hepatotoxicity is by (i) a post-administration level of said marker of hepatotoxicity greater than the upper limit of normal (ULN), or (ii) a post-administration level of said marker of hepatotoxicity greater than or equal to twice the baseline level of said marker of hepatotoxicity.

63. The method of any of claims 60-62 wherein the therapeutic immunosuppressant is a glucorticoid, optionally dexamethasone, prednisone, prednisolone, fludrocortisone, hydrocortisone, or budesonide.

64. The method of any of claims 60-62 wherein the therapeutic immunosuppressant is a glucocorticoid and the therapeutically effective amount is a prednisone-equivalent dose of from 10 mg/day to 40 mg/day, optionally for a time period of at least about 5 weeks, followed by tapering amounts of the glucocorticoid for a time period of about 3 weeks.

65. The method of any of claims 59-64 wherein the marker of hepatotoxicity is ALT and/or AST.

66. The method of any of claims 59-64 wherein the marker of hepatotoxicity is ALT.

67. The method of any of the preceding claims further comprising the step of measuring plasma functional C1-INH level of the subject every week, for at least 12 weeks.

68. A pharmaceutical composition comprising rAAV particles at a concentration of at least about 1E13 vg/ml to about 1E14 vg/ml, a tris(hydroxymethyl)aminomethane (Tris) buffering agent, an isotonicity agent, a cryopreservative agent and a surfactant which is stable during storage at about −60° C. (minus sixty degrees centigrade) or less for at least about 1 year, 1.5 years, or 2 years.

69. A pharmaceutical composition comprising rAAV particles at a concentration of at least about 1E13 vg/ml to about 1E14 vg/ml, Tris buffer, trehalose and poloxamer 188 which is stable during storage at about −60° C. (minus sixty degrees centigrade) or less for at least about 1 year, 1.5 years, or 2 years.

70. A pharmaceutical composition comprising rAAV particles at a concentration of at least about 1E13 vg/ml to about 1E14 vg/ml, a tris(hydroxymethyl)aminomethane (Tris) buffering agent, an isotonicity agent, a cryopreservative agent and a surfactant which is stable during storage at about −40° C. (minus sixty degrees centigrade) or less for at least about 1 year, 1.5 years, or 2 years.

71. A pharmaceutical composition comprising rAAV particles at a concentration of at least about 1E13 vg/ml to about 1E14 vg/ml, Tris buffer, trehalose and poloxamer 188 which is stable during storage at about −40° C. (minus sixty degrees centigrade) or less for at least about 1 year, 1.5 years, or 2 years.

72. A pharmaceutical composition comprising rAAV particles at a concentration of at least about 1E13 vg/ml to about 1E14 vg/ml, a tris(hydroxymethyl)aminomethane (Tris) buffering agent, an isotonicity agent, a cryopreservative agent and a surfactant which is stable during storage at about −20° C. (minus sixty degrees centigrade) or less for at least about 1 year, 1.5 years, or 2 years.

73. A pharmaceutical composition comprising rAAV particles at a concentration of at least about 1E13 vg/ml to about 1E14 vg/ml, Tris buffer, trehalose and poloxamer 188 which is stable during storage at about −20° C. (minus sixty degrees centigrade) or less for at least about 1 year, 1.5 years, or 2 years.

74. The formulation of any of claims 68-73 wherein the pH of the formulation ranges from about 6 to about 9, optionally ranging from about 6.8 to about 8.5.

75. The formulation of claim 74 wherein the pH of the formulation ranges from about 7 to about 7.8

76. A pharmaceutical composition comprising rAAV particles at a concentration of at least about 1E13 vg/ml to about 1E14 vg/ml, Tris buffer at a concentration of about 10 to about 30 mM, sodium chloride at a concentration of about 100 mM to about 165 mM, trehalose at a concentration of about 2 to about 3 wt %, and a poloxamer or polysorbate at a concentration of about 0.05% to about 0.15% w/v.

77. The pharmaceutical composition of claim 76 wherein the poloxamer is poloxamer 188.

78. The pharmaceutical composition of claim 76 wherein the Tris buffer is at a concentration of about 15 to about 25 mM, sodium chloride is at a concentration of about 100 to about 140 mM, trehalose is at a concentration of about 2.3 to about 2.7 wt %, and the poloxamer is poloxamer 188 at a concentration of about 0.05% to about 0.15% w/v.

79. The pharmaceutical composition of claim 78 wherein the poloxamer 188 is at a concentration of about 0.1% w/v.

80. The pharmaceutical composition of any of claims 76-79 wherein the rAAV particle is at a concentration of about 6E13 vg/ml.

81. A pharmaceutical composition which comprises rAAV particles at a concentration of about 6E13 vg/ml, about 20 mM Tris buffer, about 120 mM sodium chloride, about 2.5 wt % trehalose dihydrate, and about 0.1% w/v poloxamer 188.

82. The formulation of any of claims 76-81 wherein the pH of the formulation ranges from about 6 to about 9, optionally ranging from about 6.8 to about 8.5.

83. The formulation of claim 78 wherein the pH of the formulation ranges from about 7 to about 7.8.

84. The pharmaceutical composition of any of claims 68-83 wherein the rAAV particles comprise an AAV5 type capsid.

85. A method of using the pharmaceutical composition of any of claims 68-83 to treat a subject with hereditary angioedema by administering said pharmaceutical composition by intravenous infusion.


SEQUENCE LISTING (72 sequences)

SEQ ID NO: 1 - DNA, 1503 bp, Homo sapiens
  source 1..1503: mol_type = genomic DNA
atggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctga

SEQ ID NO: 2 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 3 - DNA, 405 bp, synthetic construct
  misc_feature 1..405: note = Synethic polynucleotide
  source 1..405: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagta

SEQ ID NO: 4 - DNA, 321 bp, synthetic construct
  misc_feature 1..321: note = Synethic polynucleotide
  source 1..321: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 5 - DNA, 725 bp, synthetic construct
  misc_feature 1..725: note = Synethic polynucleotide
  source 1..725: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 6 - DNA, 611 bp, synthetic construct
  misc_feature 1..611: note = Synethic polynucleotide
  source 1..611: mol_type = other DNA
gtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaatt

SEQ ID NO: 7 - DNA, 1129 bp, synthetic construct
  misc_feature 1..1129: note = Synethic polynucleotide
  source 1..1129: mol_type = other DNA
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgg

SEQ ID NO: 8 - DNA, 3765 bp, synthetic construct
  misc_feature 1..3765: note = Synethic polynucleotide
  source 1..3765: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaacctgcaggccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 9 - DNA, 4280 bp, synthetic construct
  misc_feature 1..4280: note = Synethic polynucleotide
  source 1..4280: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgacctcgagcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 10 - DNA, 1503 bp, synthetic construct
  misc_feature 1..1503: note = Synethic polynucleotide
  source 1..1503: mol_type = other DNA
atggcctccagactgaccctcctcacactgctcctgctgctccttgctggagatagagccagctccaaccccaatgccacaagcagcagcagccaggaccctgagagcctgcaggacagaggagagggcaaggtggccacaacagtgatctctaaaatgctcttcgttgaacccatcctggaagtgtcctctctgcctaccacaaacagcaccacaaattcagccaccaagatcacagccaataccacagatgagcccacaacccaacctaccacagaaccaacaactcagcctacaatccagcctactcagccaaccacccagctgcctacagacagccccacacagcctacaacaggcagcttctgccctggacctgtgaccctgtgcagtgatctggaatctcactctacagaggctgtgctgggagatgccctggtggacttcagcctgaaactgtatcacgccttctctgccatgaaaaaggtggagacaaatatggcttttagccctttttctattgccagcctgctgacccaagtgctgctgggagctggagagaacacaaagaccaacttggaaagcatcctgagctaccctaaggacttcacctgtgtgcaccaggccctgaagggcttcacaaccaagggagtgacaagtgtttcccaaatcttccacagccctgatctggccatcagagacacctttgtgaatgcctccagaaccctgtacagcagctctcctagagtgctgagcaacaactctgatgccaacctggaactgatcaacacctgggtggccaaaaacacaaacaacaagatcagcagactgcttgatagcctcccttctgatactagactggtcctgctgaatgctatctacctgtctgctaagtggaagaccacttttgaccccaagaaaaccagaatggaacctttccactttaagaacagtgtgatcaaggtgcctatgatgaactccaagaaataccctgtggcccacttcattgaccagaccctgaaggccaaggtgggccagctgcaactgagccacaacctgagcctggtgattctggttcctcagaacctgaagcacagactggaagatatggagcaggccctgtctccatctgtgttcaaggccatcatggagaagctggaaatgagcaagttccagcccaccctgctgacactccccagaatcaaggtgaccaccagccaggatatgctgtctatcatggaaaagcttgagttctttgacttctcctatgacctgaacctctgtggcctgacagaagatccagacctccaggtgtctgccatgcagcaccagacagtgttggagctgacagaaacaggagtggaagctgcagctgcctctgccatctctgtggccagaacactgctggtgtttgaggtgcagcagcctttcctgtttgtgctgtgggaccaacagcacaagttccctgtgtttatgggcagagtgtatgaccctagagcctga

SEQ ID NO: 11 - DNA, 1503 bp, synthetic construct
  misc_feature 1..1503: note = Synethic polynucleotide
  source 1..1503: mol_type = other DNA
atggccagcagacttaccctgcttaccctgctcttgcttctgttggctggagacagagccagctccaatcctaatgctacctccagctccagtcaagatccagagagcctgcaggatagaggagagggcaaggtggccaccacagtcataagtaaaatgctctttgtggaacccatcctggaggtgagttctcttcctaccactaactctaccacaaactctgcaaccaagatcacagccaatactacagatgagcctacaactcaaccaactacagaacccacaactcaacctacaatccagcctactcaacccactacacaactgcccactgactcacctacccagcctaccacagggtcattttgtccagggccagttaccctgtgtagtgaccttgagagccatagtacagaggctgttctgggggatgctcttgtagattttagccttaaactgtaccatgctttcagtgctatgaaaaaagttgaaacaaacatggctttttccccttttagtattgcttctctccttactcaggttcttctgggagctggggagaacacaaaaactaatcttgaatctatcctcagctatcctaaagactttacttgtgtgcaccaagctcttaaaggatttacaactaagggagttactagtgtgagtcagatttttcacagtccagaccttgctatcagagatacatttgttaatgcttcaagaactctgtatagtagcagtcccagggtcttgtcaaataacagtgatgccaatcttgagctcataaatacctgggtggctaagaatacaaacaataaaatatcaaggcttctggactctctcccaagtgataccagacttgttctcctgaatgcaatatacctcagtgctaaatggaaaactacatttgatcccaaaaaaactagaatggagccatttcactttaaaaatagtgtaatcaaggtacctatgatgaacagcaaaaaatatcctgtagctcactttattgaccaaactctcaaagccaaagtggggcaactccagctgagtcacaacctgagtttggtaattctggtaccacaaaatctgaagcacagacttgaagatatggaacaagctctctctcccagtgtttttaaagccataatggaaaagcttgaaatgagtaaatttcaacccacactcctcaccctccctaggatcaaagttaccacatcccaggacatgctcagtataatggagaaacttgagttctttgattttagctatgatctgaatctttgtggcctgacagaggatccagatctccaggtatctgccatgcagcaccagactgtgctggagctgacagaaacaggtgtagaagctgcagcagcctcagctataagtgtggctagaacattgcttgtctttgaggtccaacagccttttttgtttgttctttgggaccaacagcataaatttcctgtctttatgggaagagtttatgacccaagagcttaa

SEQ ID NO: 12 - DNA, 1503 bp, synthetic construct
  misc_feature 1..1503: note = Synethic polynucleotide
  source 1..1503: mol_type = other DNA
atggccagcagacttaccctgcttaccctgctcttgcttctgttggctggagacagagccagctccaatcctaatgctacctccagctccagtcaagatcccgagagcctgcaggatagaggagagggcaaggtggccaccacagtgatcagcaagatgctgtttgtggagcctatcctggaggtgagctctctgcctaccacaaatagcaccacaaattctgccaccaagatcacagccaataccactgatgaacccactacccagcccacaactgagcctacaacccaacctactatccaacctacccagcctaccactcaactgcccactgacagccctacccaacctacaactggctccttctgccctggccctgtgaccctgtgcagtgatctggagagccactctactgaggctgtgctgggagatgctctggtggacttcagcctgaagctgtaccacgccttctctgctatgaagaaggtggagaccaatatggctttcagccccttctctattgccagcctgctgacccaagtgctcctgggagctggagagaataccaagaccaatctggagtctatcctgagctaccccaaggatttcacctgtgtgcaccaagccctgaagggcttcaccacaaagggagtgacctctgtgagccagatcttccacagcccagatctggctatcagagatacctttgtgaatgccagcagaaccctgtacagctcttcccctagagtgctgagcaacaattctgatgctaatctggagctgatcaatacctgggtggccaagaataccaacaataagatcagcagactcctggactccctgccctctgataccagactggtgctcctgaatgctatctacctgtctgccaagtggaagaccacttttgaccccaagaagaccagaatggagcccttccacttcaagaattctgtgatcaaggtgcccatgatgaatagcaagaagtaccctgtggcccacttcattgatcagaccctgaaggctaaggtgggccaactccagctgagccacaatctcagcctggtgatcctggtgccccagaatctgaagcacagactggaggatatggagcaagccctgagcccctctgtgttcaaggctattatggaaaagcttgaaatgagcaagttccagcccacactcctgaccctgcccagaatcaaggtgaccactagccaagatatgctgtctattatggaaaaactggaattctttgacttcagctatgacctgaatctgtgtggcctgactgaagatcccgacctgcaagtgtctgccatgcagcaccaaactgtgctggagctcactgagacaggagtggaggctgcagctgcttctgccatctctgtggccagaaccctcctggtgtttgaggtgcaacagcctttcctgtttgtgctgtgggatcagcaacacaagttccctgtgttcatgggcagagtgtatgatcccagagcctaa

SEQ ID NO: 13 - DNA, 1503 bp, synthetic construct
  misc_feature 1..1503: note = Synethic polynucleotide
  source 1..1503: mol_type = other DNA
atggcttctaggctgaccctgctgactctgctgctgctgctgctggccggcgacagggcttcttctaatcccaacgccacctctagcagcagccaggatcccgagagcctgcaggacaggggggaggggaaggtggccaccaccgtgatcagcaagatgctgttcgtggagcctatcctggaggtgtctagcctgcccaccaccaactctaccaccaacagcgccaccaagattactgctaacaccaccgacgagcccaccacccagcccactaccgagcctaccactcagcccaccattcagcccacccagcctactactcagctgcccactgacagccccacccagcctaccaccggcagcttttgccccggccccgtgaccctgtgcagcgacctggagagccattctaccgaggccgtgctgggcgacgccctggtggacttcagcctgaagctgtatcatgcctttagcgctatgaagaaggtggagactaacatggccttcagccccttctctatcgcctctctgctgacccaggtgctgctgggcgccggggagaacaccaagaccaatctggagagcatcctgagctaccccaaggatttcacttgcgtgcaccaggctctgaagggcttcaccaccaagggcgtgactagcgtgtctcagatcttccactctcccgacctggccattagggacactttcgtgaacgccagcaggactctgtacagcagcagccccagggtgctgagcaacaatagcgacgctaacctggagctgatcaacacctgggtggccaagaacaccaacaacaagatcagcaggctgctggacagcctgcccagcgataccaggctggtgctgctgaacgctatctacctgtctgctaagtggaaaactaccttcgatcctaagaaaaccaggatggagcccttccatttcaagaatagcgtgatcaaggtgcccatgatgaacagcaagaagtaccccgtggcccacttcatcgaccagaccctgaaggccaaggtgggccagctgcagctgagccacaacctgagcctggtgatcctggtgccccagaatctgaagcacaggctggaggacatggagcaggccctgagccccagcgtgttcaaggccatcatggagaagctggagatgtctaagttccagcccaccctgctgaccctgcccaggatcaaggtgaccactagccaggacatgctgagcattatggagaagctggagttcttcgacttctcttacgacctgaacctgtgcggcctgactgaggatcccgatctgcaggtgtctgctatgcagcaccagaccgtgctggagctgaccgagactggcgtggaggccgccgccgccagcgccatcagcgtggccaggaccctgctggtgttcgaggtgcagcagccctttctgtttgtgctgtgggatcagcagcacaagttccctgtgttcatggggagagtgtacgaccctagggcctga

SEQ ID NO: 14 - DNA, 32 bp, synthetic construct
  misc_feature 1..32: note = Synethic polynucleotide
  source 1..32: mol_type = other DNA
tggacacaggacgctgtggtttctgagccagg

SEQ ID NO: 15 - DNA, 186 bp, synthetic construct
  misc_feature 1..186: note = Synethic polynucleotide
  source 1..186: mol_type = other DNA
gggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 16 - DNA, 34 bp, synthetic construct
  misc_feature 1..34: note = Synethic polynucleotide
  source 1..34: mol_type = other DNA
tgtttgctgcttgcaatgtttgcccattttaggg

SEQ ID NO: 17 - DNA, 351 bp, synthetic construct
  misc_feature 1..351: note = Synethic polynucleotide
  source 1..351: mol_type = other DNA
gtcgacaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggagaggctcaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggagaggctgtcgac

SEQ ID NO: 18 - DNA, 252 bp, synthetic construct
  misc_feature 1..252: note = Synethic polynucleotide
  source 1..252: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 19 - DNA, 227 bp, synthetic construct
  misc_feature 1..227: note = Synethic polynucleotide
  source 1..227: mol_type = other DNA
agctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 20 - DNA, 3075 bp, synthetic construct
  misc_feature 1..3075: note = Synethic polynucleotide
  source 1..3075: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 21 - DNA, 3134 bp, synthetic construct
  misc_feature 1..3134: note = Synethic polynucleotide
  source 1..3134: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 22 - DNA, 3471 bp, synthetic construct
  misc_feature 1..3471: note = Synethic polynucleotide
  source 1..3471: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 23 - DNA, 3900 bp, synthetic construct
  misc_feature 1..3900: note = Synethic polynucleotide
  source 1..3900: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 24 - DNA, 3810 bp, synthetic construct
  misc_feature 1..3810: note = Synethic polynucleotide
  source 1..3810: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagcaggctcactctgctgactctgctgctgctgctgctggctggggacagggccagcagcaaccccaatgccaccagcagcagcagccaggaccctgagagcctccaggacaggggggagggcaaggtggccaccactgtgatcagcaagatgctgtttgtggagcccatcctggaggtcagcagcctgcccaccaccaacagcaccaccaactctgccaccaagatcactgccaacactactgatgaacccactacccaacccaccactgagcccaccactcagcccactatccagcccacccagcccaccacccagctgcccactgacagccccacccagcccaccactggcagcttctgccctggccctgtgactctgtgcagtgatctggagagccacagcactgaggctgtgctgggggatgccctggtggacttcagcctgaagctgtaccacgccttcagtgctatgaagaaggtggagaccaatatggccttcagccccttcagtattgccagcctgctgactcaggtgctgctgggggctggggagaacactaagaccaacctggagagtatcctgagctaccccaaggacttcacttgtgtgcaccaggccctgaagggcttcactaccaagggggtcactagtgtgagccagatcttccacagccctgatctggccatcagggacacttttgtgaatgccagcaggaccctgtacagcagcagccccagggtgctgagcaacaacagtgatgccaacctggagctgatcaatacctgggtggccaagaataccaacaacaagatcagcaggctgctggacagcctgcccagtgataccaggctggtgctgctgaatgctatctacctgagtgccaagtggaagaccacctttgatcccaagaagaccaggatggagcccttccacttcaagaacagtgtgatcaaggtgcccatgatgaacagcaagaagtaccctgtggcccacttcattgatcagaccctgaaggccaaggtgggccagctgcagctgagccacaacctgagcctggtcatcctggtgccccagaacctgaagcacaggctggaggatatggagcaggccctgagccccagtgtgttcaaggctattatggagaagctcgaaatgagcaagttccagcccaccctgctgactctgcccaggatcaaggtcactaccagccaggatatgctgtctattatggagaaactggagttctttgacttcagctatgatctgaacctgtgtgggctgactgaggaccctgatctgcaagtgagtgccatgcagcaccagactgtgctggagctgactgagactggggtggaggctgctgctgctagtgccatcagtgtggccaggaccctgctggtctttgaggtgcagcagcccttcctgtttgtgctgtgggaccagcagcacaagttccctgtgttcatgggcagggtctatgatcccagggcctaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 25 - DNA, 3810 bp, synthetic construct
  misc_feature 1..3810: note = Synethic polynucleotide
  source 1..3810: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagccgcctgaccctgctgaccctgctgctgctgctgctggccggcgaccgcgccagcagcaaccccaacgccaccagcagcagcagccaggaccccgagagcctgcaggaccgcggcgagggcaaggtggccaccaccgtgatcagcaagatgctgttcgtggagcccatcctggaggtgagcagcctgcccaccaccaacagcaccaccaacagcgccaccaagatcaccgccaacaccaccgacgagcccaccacccagcccaccaccgagcccaccacccagcccaccatccagcccacccagcccaccacccagctgcccaccgacagccccacccagcccaccaccggcagcttctgccccggccccgtgaccctgtgcagcgacctggagagccacagcaccgaggccgtgctgggcgacgccctggtggacttcagcctgaagctgtaccacgccttcagcgccatgaagaaggtggagaccaacatggccttcagccccttcagcatcgccagcctgctgacccaggtgctgctgggcgccggcgagaacaccaagaccaacctggagagcatcctgagctaccccaaggacttcacctgcgtgcaccaggccctgaagggcttcaccaccaagggcgtgaccagcgtgagccagatcttccacagccccgacctggccatccgcgacaccttcgtgaacgccagccgcaccctgtacagcagcagcccccgcgtgctgagcaacaacagcgacgccaacctggagctgatcaacacctgggtggccaagaacaccaacaacaagatcagccgcctgctggacagcctgcccagcgacacccgcctggtgctgctgaacgccatctacctgagcgccaagtggaagaccaccttcgaccccaagaagacccgcatggagcccttccacttcaagaacagcgtgatcaaggtgcccatgatgaacagcaagaagtaccccgtggcccacttcatcgaccagaccctgaaggccaaggtgggccagctgcagctgagccacaacctgagcctggtgatcctggtgccccagaacctgaagcaccgcctggaggacatggagcaggccctgagccccagcgtgttcaaggccatcatggagaagctggagatgagcaagttccagcccaccctgctgaccctgccccgcatcaaggtgaccaccagccaggacatgctgagcatcatggagaagctggagttcttcgacttcagctacgacctgaacctgtgcgggctgaccgaggaccccgacctgcaggtgagcgccatgcagcaccagaccgtgctggagctgaccgagaccggcgtggaggccgccgccgccagcgccatcagcgtggcccgcaccctgctggtgttcgaggtgcagcagcccttcctgttcgtgctgtgggaccagcagcacaagttccccgtgttcatgggccgcgtgtacgacccccgcgcctaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 26 - DNA, 3810 bp, synthetic construct
  misc_feature 1..3810: note = Synethic polynucleotide
  source 1..3810: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcttctaggctgaccctgctgactctgctgctgctgctgctggccggcgacagggcttcttctaatcccaacgccacctctagcagcagccaggatcccgagagcctgcaggacaggggggaggggaaggtggccaccaccgtgatcagcaagatgctgttcgtggagcctatcctggaggtgtctagcctgcccaccaccaactctaccaccaacagcgccaccaagattactgctaacaccaccgacgagcccaccacccagcccactaccgagcctaccactcagcccaccattcagcccacccagcctactactcagctgcccactgacagccccacccagcctaccaccggcagcttttgccccggccccgtgaccctgtgcagcgacctggagagccattctaccgaggccgtgctgggcgacgccctggtggacttcagcctgaagctgtatcatgcctttagcgctatgaagaaggtggagactaacatggccttcagccccttctctatcgcctctctgctgacccaggtgctgctgggcgccggggagaacaccaagaccaatctggagagcatcctgagctaccccaaggatttcacttgcgtgcaccaggctctgaagggcttcaccaccaagggcgtgactagcgtgtctcagatcttccactctcccgacctggccattagggacactttcgtgaacgccagcaggactctgtacagcagcagccccagggtgctgagcaacaatagcgacgctaacctggagctgatcaacacctgggtggccaagaacaccaacaacaagatcagcaggctgctggacagcctgcccagcgataccaggctggtgctgctgaacgctatctacctgtctgctaagtggaaaactaccttcgatcctaagaaaaccaggatggagcccttccatttcaagaatagcgtgatcaaggtgcccatgatgaacagcaagaagtaccccgtggcccacttcatcgaccagaccctgaaggccaaggtgggccagctgcagctgagccacaacctgagcctggtgatcctggtgccccagaatctgaagcacaggctggaggacatggagcaggccctgagccccagcgtgttcaaggccatcatggagaagctggagatgtctaagttccagcccaccctgctgaccctgcccaggatcaaggtgaccactagccaggacatgctgagcattatggagaagctggagttcttcgacttctcttacgacctgaacctgtgcggcctgactgaggatcccgatctgcaggtgtctgctatgcagcaccagaccgtgctggagctgaccgagactggcgtggaggccgccgccgccagcgccatcagcgtggccaggaccctgctggtgttcgaggtgcagcagccctttctgtttgtgctgtgggatcagcagcacaagttccctgtgttcatggggagagtgtacgaccctagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 27 - DNA, 4767 bp, synthetic construct
  misc_feature 1..4767: note = Synethic polynucleotide
  source 1..4767: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagccgcctgaccctgctgaccctgctgctgctgctgctggccggcgaccgcgccagcagcaaccccaacgccaccagcagcagcagccaggaccccgagagcctgcaggaccgcggcgagggcaaggtggccaccaccgtgatcagcaagatgctgttcgtggagcccatcctggaggtgagcagcctgcccaccaccaacagcaccaccaacagcgccaccaagatcaccgccaacaccaccgacgagcccaccacccagcccaccaccgagcccaccacccagcccaccatccagcccacccagcccaccacccagctgcccaccgacagccccacccagcccaccaccggcagcttctgccccggccccgtgaccctgtgcagcgacctggagagccacagcaccgaggccgtgctgggcgacgccctggtggacttcagcctgaagctgtaccacgccttcagcgccatgaagaaggtggagaccaacatggccttcagccccttcagcatcgccagcctgctgacccaggtgctgctgggcgccggcgagaacaccaagaccaacctggagagcatcctgagctaccccaaggacttcacctgcgtgcaccaggccctgaagggcttcaccaccaagggcgtgaccagcgtgagccagatcttccacagccccgacctggccatccgcgacaccttcgtgaacgccagccgcaccctgtacagcagcagcccccgcgtgctgagcaacaacagcgacgccaacctggagctgatcaacacctgggtggccaagaacaccaacaacaagatcagccgcctgctggacagcctgcccagcgacacccgcctggtgctgctgaacgccatctacctgagcgccaagtggaagaccaccttcgaccccaagaagacccgcatggagcccttccacttcaagaacagcgtgatcaaggtgcccatgatgaacagcaagaagtaccccgtggcccacttcatcgaccagaccctgaaggccaaggtgggccagctgcagctgagccacaacctgagcctggtgatcctggtgccccagaacctgaagcaccgcctggaggacatggagcaggccctgagccccagcgtgttcaaggccatcatggagaagctggagatgagcaagttccagcccaccctgctgaccctgccccgcatcaaggtgaccaccagccaggacatgctgagcatcatggagaagctggagttcttcgacttcagctacgacctgaacctgtgcgggctgaccgaggaccccgacctgcaggtgagcgccatgcagcaccagaccgtgctggagctgaccgagaccggcgtggaggccgccgccgccagcgccatcagcgtggcccgcaccctgctggtgttcgaggtgcagcagcccttcctgttcgtgctgtgggaccagcagcacaagttccccgtgttcatgggccgcgtgtacgacccccgcgcctaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 28 - DNA, 4767 bp, synthetic construct
  misc_feature 1..4767: note = Synethic polynucleotide
  source 1..4767: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccagactgaccctcctcacactgctcctgctgctccttgctggagatagagccagctccaaccccaatgccacaagcagcagcagccaggaccctgagagcctgcaggacagaggagagggcaaggtggccacaacagtgatctctaaaatgctcttcgttgaacccatcctggaagtgtcctctctgcctaccacaaacagcaccacaaattcagccaccaagatcacagccaataccacagatgagcccacaacccaacctaccacagaaccaacaactcagcctacaatccagcctactcagccaaccacccagctgcctacagacagccccacacagcctacaacaggcagcttctgccctggacctgtgaccctgtgcagtgatctggaatctcactctacagaggctgtgctgggagatgccctggtggacttcagcctgaaactgtatcacgccttctctgccatgaaaaaggtggagacaaatatggcttttagccctttttctattgccagcctgctgacccaagtgctgctgggagctggagagaacacaaagaccaacttggaaagcatcctgagctaccctaaggacttcacctgtgtgcaccaggccctgaagggcttcacaaccaagggagtgacaagtgtttcccaaatcttccacagccctgatctggccatcagagacacctttgtgaatgcctccagaaccctgtacagcagctctcctagagtgctgagcaacaactctgatgccaacctggaactgatcaacacctgggtggccaaaaacacaaacaacaagatcagcagactgcttgatagcctcccttctgatactagactggtcctgctgaatgctatctacctgtctgctaagtggaagaccacttttgaccccaagaaaaccagaatggaacctttccactttaagaacagtgtgatcaaggtgcctatgatgaactccaagaaataccctgtggcccacttcattgaccagaccctgaaggccaaggtgggccagctgcaactgagccacaacctgagcctggtgattctggttcctcagaacctgaagcacagactggaagatatggagcaggccctgtctccatctgtgttcaaggccatcatggagaagctggaaatgagcaagttccagcccaccctgctgacactccccagaatcaaggtgaccaccagccaggatatgctgtctatcatggaaaagcttgagttctttgacttctcctatgacctgaacctctgtggcctgacagaagatccagacctccaggtgtctgccatgcagcaccagacagtgttggagctgacagaaacaggagtggaagctgcagctgcctctgccatctctgtggccagaacactgctggtgtttgaggtgcagcagcctttcctgtttgtgctgtgggaccaacagcacaagttccctgtgtttatgggcagagtgtatgaccctagagcctgacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 29 - DNA, 4767 bp, synthetic construct
  misc_feature 1..4767: note = Synethic polynucleotide
  source 1..4767: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagcagacttaccctgcttaccctgctcttgcttctgttggctggagacagagccagctccaatcctaatgctacctccagctccagtcaagatccagagagcctgcaggatagaggagagggcaaggtggccaccacagtcataagtaaaatgctctttgtggaacccatcctggaggtgagttctcttcctaccactaactctaccacaaactctgcaaccaagatcacagccaatactacagatgagcctacaactcaaccaactacagaacccacaactcaacctacaatccagcctactcaacccactacacaactgcccactgactcacctacccagcctaccacagggtcattttgtccagggccagttaccctgtgtagtgaccttgagagccatagtacagaggctgttctgggggatgctcttgtagattttagccttaaactgtaccatgctttcagtgctatgaaaaaagttgaaacaaacatggctttttccccttttagtattgcttctctccttactcaggttcttctgggagctggggagaacacaaaaactaatcttgaatctatcctcagctatcctaaagactttacttgtgtgcaccaagctcttaaaggatttacaactaagggagttactagtgtgagtcagatttttcacagtccagaccttgctatcagagatacatttgttaatgcttcaagaactctgtatagtagcagtcccagggtcttgtcaaataacagtgatgccaatcttgagctcataaatacctgggtggctaagaatacaaacaataaaatatcaaggcttctggactctctcccaagtgataccagacttgttctcctgaatgcaatatacctcagtgctaaatggaaaactacatttgatcccaaaaaaactagaatggagccatttcactttaaaaatagtgtaatcaaggtacctatgatgaacagcaaaaaatatcctgtagctcactttattgaccaaactctcaaagccaaagtggggcaactccagctgagtcacaacctgagtttggtaattctggtaccacaaaatctgaagcacagacttgaagatatggaacaagctctctctcccagtgtttttaaagccataatggaaaagcttgaaatgagtaaatttcaacccacactcctcaccctccctaggatcaaagttaccacatcccaggacatgctcagtataatggagaaacttgagttctttgattttagctatgatctgaatctttgtggcctgacagaggatccagatctccaggtatctgccatgcagcaccagactgtgctggagctgacagaaacaggtgtagaagctgcagcagcctcagctataagtgtggctagaacattgcttgtctttgaggtccaacagccttttttgtttgttctttgggaccaacagcataaatttcctgtctttatgggaagagtttatgacccaagagcttaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 30 - DNA, 4767 bp, synthetic construct
  misc_feature 1..4767: note = Synethic polynucleotide
  source 1..4767: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagcagacttaccctgcttaccctgctcttgcttctgttggctggagacagagccagctccaatcctaatgctacctccagctccagtcaagatcccgagagcctgcaggatagaggagagggcaaggtggccaccacagtgatcagcaagatgctgtttgtggagcctatcctggaggtgagctctctgcctaccacaaatagcaccacaaattctgccaccaagatcacagccaataccactgatgaacccactacccagcccacaactgagcctacaacccaacctactatccaacctacccagcctaccactcaactgcccactgacagccctacccaacctacaactggctccttctgccctggccctgtgaccctgtgcagtgatctggagagccactctactgaggctgtgctgggagatgctctggtggacttcagcctgaagctgtaccacgccttctctgctatgaagaaggtggagaccaatatggctttcagccccttctctattgccagcctgctgacccaagtgctcctgggagctggagagaataccaagaccaatctggagtctatcctgagctaccccaaggatttcacctgtgtgcaccaagccctgaagggcttcaccacaaagggagtgacctctgtgagccagatcttccacagcccagatctggctatcagagatacctttgtgaatgccagcagaaccctgtacagctcttcccctagagtgctgagcaacaattctgatgctaatctggagctgatcaatacctgggtggccaagaataccaacaataagatcagcagactcctggactccctgccctctgataccagactggtgctcctgaatgctatctacctgtctgccaagtggaagaccacttttgaccccaagaagaccagaatggagcccttccacttcaagaattctgtgatcaaggtgcccatgatgaatagcaagaagtaccctgtggcccacttcattgatcagaccctgaaggctaaggtgggccaactccagctgagccacaatctcagcctggtgatcctggtgccccagaatctgaagcacagactggaggatatggagcaagccctgagcccctctgtgttcaaggctattatggaaaagcttgaaatgagcaagttccagcccacactcctgaccctgcccagaatcaaggtgaccactagccaagatatgctgtctattatggaaaaactggaattctttgacttcagctatgacctgaatctgtgtggcctgactgaagatcccgacctgcaagtgtctgccatgcagcaccaaactgtgctggagctcactgagacaggagtggaggctgcagctgcttctgccatctctgtggccagaaccctcctggtgtttgaggtgcaacagcctttcctgtttgtgctgtgggatcagcaacacaagttccctgtgttcatgggcagagtgtatgatcccagagcctaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 31 - DNA, 4280 bp, synthetic construct
  misc_feature 1..4280: note = Synethic polynucleotide
  source 1..4280: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccagactgaccctcctcacactgctcctgctgctccttgctggagatagagccagctccaaccccaatgccacaagcagcagcagccaggaccctgagagcctgcaggacagaggagagggcaaggtggccacaacagtgatctctaaaatgctcttcgttgaacccatcctggaagtgtcctctctgcctaccacaaacagcaccacaaattcagccaccaagatcacagccaataccacagatgagcccacaacccaacctaccacagaaccaacaactcagcctacaatccagcctactcagccaaccacccagctgcctacagacagccccacacagcctacaacaggcagcttctgccctggacctgtgaccctgtgcagtgatctggaatctcactctacagaggctgtgctgggagatgccctggtggacttcagcctgaaactgtatcacgccttctctgccatgaaaaaggtggagacaaatatggcttttagccctttttctattgccagcctgctgacccaagtgctgctgggagctggagagaacacaaagaccaacttggaaagcatcctgagctaccctaaggacttcacctgtgtgcaccaggccctgaagggcttcacaaccaagggagtgacaagtgtttcccaaatcttccacagccctgatctggccatcagagacacctttgtgaatgcctccagaaccctgtacagcagctctcctagagtgctgagcaacaactctgatgccaacctggaactgatcaacacctgggtggccaaaaacacaaacaacaagatcagcagactgcttgatagcctcccttctgatactagactggtcctgctgaatgctatctacctgtctgctaagtggaagaccacttttgaccccaagaaaaccagaatggaacctttccactttaagaacagtgtgatcaaggtgcctatgatgaactccaagaaataccctgtggcccacttcattgaccagaccctgaaggccaaggtgggccagctgcaactgagccacaacctgagcctggtgattctggttcctcagaacctgaagcacagactggaagatatggagcaggccctgtctccatctgtgttcaaggccatcatggagaagctggaaatgagcaagttccagcccaccctgctgacactccccagaatcaaggtgaccaccagccaggatatgctgtctatcatggaaaagcttgagttctttgacttctcctatgacctgaacctctgtggcctgacagaagatccagacctccaggtgtctgccatgcagcaccagacagtgttggagctgacagaaacaggagtggaagctgcagctgcctctgccatctctgtggccagaacactgctggtgtttgaggtgcagcagcctttcctgtttgtgctgtgggaccaacagcacaagttccctgtgtttatgggcagagtgtatgaccctagagcctgacctcgagcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 32 - DNA, 4280 bp, synthetic construct
  misc_feature 1..4280: note = Synethic polynucleotide
  source 1..4280: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagcagacttaccctgcttaccctgctcttgcttctgttggctggagacagagccagctccaatcctaatgctacctccagctccagtcaagatccagagagcctgcaggatagaggagagggcaaggtggccaccacagtcataagtaaaatgctctttgtggaacccatcctggaggtgagttctcttcctaccactaactctaccacaaactctgcaaccaagatcacagccaatactacagatgagcctacaactcaaccaactacagaacccacaactcaacctacaatccagcctactcaacccactacacaactgcccactgactcacctacccagcctaccacagggtcattttgtccagggccagttaccctgtgtagtgaccttgagagccatagtacagaggctgttctgggggatgctcttgtagattttagccttaaactgtaccatgctttcagtgctatgaaaaaagttgaaacaaacatggctttttccccttttagtattgcttctctccttactcaggttcttctgggagctggggagaacacaaaaactaatcttgaatctatcctcagctatcctaaagactttacttgtgtgcaccaagctcttaaaggatttacaactaagggagttactagtgtgagtcagatttttcacagtccagaccttgctatcagagatacatttgttaatgcttcaagaactctgtatagtagcagtcccagggtcttgtcaaataacagtgatgccaatcttgagctcataaatacctgggtggctaagaatacaaacaataaaatatcaaggcttctggactctctcccaagtgataccagacttgttctcctgaatgcaatatacctcagtgctaaatggaaaactacatttgatcccaaaaaaactagaatggagccatttcactttaaaaatagtgtaatcaaggtacctatgatgaacagcaaaaaatatcctgtagctcactttattgaccaaactctcaaagccaaagtggggcaactccagctgagtcacaacctgagtttggtaattctggtaccacaaaatctgaagcacagacttgaagatatggaacaagctctctctcccagtgtttttaaagccataatggaaaagcttgaaatgagtaaatttcaacccacactcctcaccctccctaggatcaaagttaccacatcccaggacatgctcagtataatggagaaacttgagttctttgattttagctatgatctgaatctttgtggcctgacagaggatccagatctccaggtatctgccatgcagcaccagactgtgctggagctgacagaaacaggtgtagaagctgcagcagcctcagctataagtgtggctagaacattgcttgtctttgaggtccaacagccttttttgtttgttctttgggaccaacagcataaatttcctgtctttatgggaagagtttatgacccaagagcttaacctcgagcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 33 - DNA, 4280 bp, synthetic construct
  misc_feature 1..4280: note = Synethic polynucleotide
  source 1..4280: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggccagcagacttaccctgcttaccctgctcttgcttctgttggctggagacagagccagctccaatcctaatgctacctccagctccagtcaagatcccgagagcctgcaggatagaggagagggcaaggtggccaccacagtgatcagcaagatgctgtttgtggagcctatcctggaggtgagctctctgcctaccacaaatagcaccacaaattctgccaccaagatcacagccaataccactgatgaacccactacccagcccacaactgagcctacaacccaacctactatccaacctacccagcctaccactcaactgcccactgacagccctacccaacctacaactggctccttctgccctggccctgtgaccctgtgcagtgatctggagagccactctactgaggctgtgctgggagatgctctggtggacttcagcctgaagctgtaccacgccttctctgctatgaagaaggtggagaccaatatggctttcagccccttctctattgccagcctgctgacccaagtgctcctgggagctggagagaataccaagaccaatctggagtctatcctgagctaccccaaggatttcacctgtgtgcaccaagccctgaagggcttcaccacaaagggagtgacctctgtgagccagatcttccacagcccagatctggctatcagagatacctttgtgaatgccagcagaaccctgtacagctcttcccctagagtgctgagcaacaattctgatgctaatctggagctgatcaatacctgggtggccaagaataccaacaataagatcagcagactcctggactccctgccctctgataccagactggtgctcctgaatgctatctacctgtctgccaagtggaagaccacttttgaccccaagaagaccagaatggagcccttccacttcaagaattctgtgatcaaggtgcccatgatgaatagcaagaagtaccctgtggcccacttcattgatcagaccctgaaggctaaggtgggccaactccagctgagccacaatctcagcctggtgatcctggtgccccagaatctgaagcacagactggaggatatggagcaagccctgagcccctctgtgttcaaggctattatggaaaagcttgaaatgagcaagttccagcccacactcctgaccctgcccagaatcaaggtgaccactagccaagatatgctgtctattatggaaaaactggaattctttgacttcagctatgacctgaatctgtgtggcctgactgaagatcccgacctgcaagtgtctgccatgcagcaccaaactgtgctggagctcactgagacaggagtggaggctgcagctgcttctgccatctctgtggccagaaccctcctggtgtttgaggtgcaacagcctttcctgtttgtgctgtgggatcagcaacacaagttccctgtgttcatgggcagagtgtatgatcccagagcctaacctcgagcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 34 - DNA, 4767 bp, synthetic construct
  misc_feature 1..4767: note = Synethic polynucleotide
  source 1..4767: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 35 - DNA, 4385 bp, synthetic construct
  misc_feature 1..4385: note = Synethic polynucleotide
  source 1..4385: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagtttttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 36 - DNA, 3765 bp, synthetic construct; identical to SEQ ID NO: 8
  misc_feature 1..3765: note = Synethic polynucleotide
  source 1..3765: mol_type = other DNA

SEQ ID NO: 37 - protein, 736 aa, Adeno-Associated Virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - protein, 736 aa, Adeno-Associated Virus Rh.8
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 39 - protein, 738 aa, Adeno-Associated Virus Rh.10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 40 - protein, 738 aa, Adeno-Associated Virus Rh.74
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 41 - protein, 736 aa, Adeno-Associated Virus 1
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 42 - protein, 735 aa, Adeno-Associated Virus 2 [sequence omitted]

SEQ ID NO: 43 - protein, 736 aa, Adeno-Associated Virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 44 - protein, 736 aa, Adeno-Associated Virus 3B; identical to SEQ ID NO: 41

SEQ ID NO: 45 - protein, 734 aa, Adeno-Associated Virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 46 - protein, 724 aa, Adeno-Associated Virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 47 - protein, 736 aa, Adeno-Associated Virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 48 - protein, 737 aa, Adeno-Associated Virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 49 - protein, 738 aa, Adeno-Associated Virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 50 - protein, 738 aa, Adeno-Associated Virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 51 - protein, 733 aa, Adeno-Associated Virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 52 - protein, 742 aa, Adeno-Associated Virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 53 - protein, 733 aa, Adeno-Associated Virus 13
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 54 - DNA, 130 bp, Adeno-Associated Virus
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 55 - DNA, 130 bp, Adeno-Associated Virus
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 56 - protein, 742 aa, synthetic construct
  REGION 1..742: note = Synthetic polypeptide
MAADGYLPDWLEDNLSEGIREWWALKPGAPQPKANQQHQDNARGLVLPGYKYLGPGNGLDKGEPVNEADAAALEHDKAYDQQLKSGDNPYLKYNHADAEFQERLQEDTSFGGNLGRAVFQAKKRILEPLGLVEEGVKTAPGKKRPLEKTPNRPTNPDSGKAPAKKKQKDGETADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSLPPFPNDVFMVPQYGYCGVVTGENQNQTDRNAFYCLEYFPSQMLRTGNNFEISYQFEKVPFHSMYAHSQSLDRMMNPLLDQYLWHLQSTTTGNSLNQGTAITTYGKITTGDFAYYRKNWLPGAGIKQQKFSKNASQNYKIPASGGDALLKYDTHTTLNGRWSNMAPGPPMATAGAGDSDFSNSQLIFAGPNQSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHEPRAIGSRFLTHHL

SEQ ID NO: 57 - DNA, 2954 bp, synthetic construct
  misc_feature 1..2954: note = Synthetic polynucleotide
  source 1..2954: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 58 - DNA, 3853 bp, synthetic construct
  misc_feature 1..3853: note = Synthetic polynucleotide
  source 1..3853: mol_type = other DNA
ggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgacctcgagcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagagcttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcc

SEQ ID NO: 59 - DNA, 1508 bp, synthetic construct
  misc_feature 1..1508: note = Synthetic polynucleotide
  source 1..1508: mol_type = other DNA
atggccagcaggctcactctgctgactctgctgctgctgctgctggctggggacagggccagcagcaaccccaatgccaccagcagcagcagccaggaccctgagagcctccaggacaggggggagggcaaggtggccaccactgtgatcagcaagatgctgtttgtggagcccatcctggaggtcagcagcctgcccaccaccaacagcaccaccaactctgccaccaagatcactgccaacactactgatgaacccactacccaacccaccactgagcccaccactcagcccactatccagcccacccagcccaccacccagctgcccactgacagccccacccagcccaccactggcagcttctgccctggccctgtgactctgtgcagtgatctggagagccacagcactgaggctgtgctgggggatgccctggtggacttcagcctgaagctgtaccacgccttcagtgctatgaagaaggtggagaccaatatggccttcagccccttcagtattgccagcctgctgactcaggtgctgctgggggctggggagaacactaagaccaacctggagagtatcctgagctaccccaaggacttcacttgtgtgcaccaggccctgaagggcttcactaccaagggggtcactagtgtgagccagatcttccacagccctgatctggccatcagggacacttttgtgaatgccagcaggaccctgtacagcagcagccccagggtgctgagcaacaacagtgatgccaacctggagctgatcaatacctgggtggccaagaataccaacaacaagatcagcaggctgctggacagcctgcccagtgataccaggctggtgctgctgaatgctatctacctgagtgccaagtggaagaccacctttgatcccaagaagaccaggatggagcccttccacttcaagaacagtgtgatcaaggtgcccatgatgaacagcaagaagtaccctgtggcccacttcattgatcagaccctgaaggccaaggtgggccagctgcagctgagccacaacctgagcctggtcatcctggtgccccagaacctgaagcacaggctggaggatatggagcaggccctgagccccagtgtgttcaaggctattatggagaagctcgaaatgagcaagttccagcccaccctgctgactctgcccaggatcaaggtcactaccagccaggatatgctgtctattatggagaaactggagttctttgacttcagctatgatctgaacctgtgtgggctgactgaggaccctgatctgcaagtgagtgccatgcagcaccagactgtgctggagctgactgagactggggtggaggctgctgctgctagtgccatcagtgtggccaggaccctgctggtctttgaggtgcagcagcccttcctgtttgtgctgtgggaccagcagcacaagttccctgtgttcatgggcagggtctatgatcccagggcctaacctcg

SEQ ID NO: 60 - DNA, 1508 bp, synthetic construct
  misc_feature 1..1508: note = Synthetic polynucleotide
  source 1..1508: mol_type = other DNA
atggccagccgcctgaccctgctgaccctgctgctgctgctgctggccggcgaccgcgccagcagcaaccccaacgccaccagcagcagcagccaggaccccgagagcctgcaggaccgcggcgagggcaaggtggccaccaccgtgatcagcaagatgctgttcgtggagcccatcctggaggtgagcagcctgcccaccaccaacagcaccaccaacagcgccaccaagatcaccgccaacaccaccgacgagcccaccacccagcccaccaccgagcccaccacccagcccaccatccagcccacccagcccaccacccagctgcccaccgacagccccacccagcccaccaccggcagcttctgccccggccccgtgaccctgtgcagcgacctggagagccacagcaccgaggccgtgctgggcgacgccctggtggacttcagcctgaagctgtaccacgccttcagcgccatgaagaaggtggagaccaacatggccttcagccccttcagcatcgccagcctgctgacccaggtgctgctgggcgccggcgagaacaccaagaccaacctggagagcatcctgagctaccccaaggacttcacctgcgtgcaccaggccctgaagggcttcaccaccaagggcgtgaccagcgtgagccagatcttccacagccccgacctggccatccgcgacaccttcgtgaacgccagccgcaccctgtacagcagcagcccccgcgtgctgagcaacaacagcgacgccaacctggagctgatcaacacctgggtggccaagaacaccaacaacaagatcagccgcctgctggacagcctgcccagcgacacccgcctggtgctgctgaacgccatctacctgagcgccaagtggaagaccaccttcgaccccaagaagacccgcatggagcccttccacttcaagaacagcgtgatcaaggtgcccatgatgaacagcaagaagtaccccgtggcccacttcatcgaccagaccctgaaggccaaggtgggccagctgcagctgagccacaacctgagcctggtgatcctggtgccccagaacctgaagcaccgcctggaggacatggagcaggccctgagccccagcgtgttcaaggccatcatggagaagctggagatgagcaagttccagcccaccctgctgaccctgccccgcatcaaggtgaccaccagccaggacatgctgagcatcatggagaagctggagttcttcgacttcagctacgacctgaacctgtgcgggctgaccgaggaccccgacctgcaggtgagcgccatgcagcaccagaccgtgctggagctgaccgagaccggcgtggaggccgccgccgccagcgccatcagcgtggcccgcaccctgctggtgttcgaggtgcagcagcccttcctgttcgtgctgtgggaccagcagcacaagttccccgtgttcatgggccgcgtgtacgacccccgcgcctaacctcg

SEQ ID NO: 61 - DNA, 196 bp, synthetic construct
  misc_feature 1..196: note = Synthetic polynucleotide
  source 1..196: mol_type = other DNA
gtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 62 - DNA, 317 bp, synthetic construct
  misc_feature 1..317: note = Synthetic polynucleotide
  source 1..317: mol_type = other DNA
gtaagtatgcctttcactgcgaggggttctggagaggcttagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccacc

SEQ ID NO: 63 - DNA, 376 bp, synthetic construct
  misc_feature 1..376: note = Synthetic polynucleotide
  source 1..376: mol_type = other DNA
gtaagtatgcctttcactgcgaggggttctggagaggcttctgagctccccatggcccaggcaggcagcaggtctggggcaggaggggggttgtggagtagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgttcatgccttcttctttttcctacagctcctgggcaacgtgctggttgttgtgctgtctcatcattttggcaaacctgcaggccacc

SEQ ID NO: 64 - DNA, 435 bp, synthetic construct
  misc_feature 1..435: note = Synthetic polynucleotide
  source 1..435: mol_type = other DNA
gtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggca

SEQ ID NO: 65 - DNA, 864 bp, synthetic construct
  misc_feature 1..864: note = Synthetic polynucleotide
  source 1..864: mol_type = other DNA
gtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactct

SEQ ID NO: 66 - DNA, 1439 bp, synthetic construct
  misc_feature 1..1439: note = Synthetic polynucleotide
  source 1..1439: mol_type = other DNA
gtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagtttt

SEQ ID NO: 67 - DNA, 1821 bp, synthetic construct
  misc_feature 1..1821: note = Synthetic polynucleotide
  source 1..1821: mol_type = other DNA
gtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgc

SEQ ID NO: 68 - DNA, 130 bp, synthetic construct
  misc_feature 1..130: note = Synthetic polynucleotide
  source 1..130: mol_type = other DNA
gtaagtatgcctttcactgcgagaggttctggagaggcttctgagctccccatggcccaggcaggcagcaggtctggggcaggaggggggttgtggagtgggtatccgcctgctgaggtgcagggcagat

SEQ ID NO: 69 - DNA, 98 bp, synthetic construct
  misc_feature 1..98: note = Synthetic polynucleotide
  source 1..98: mol_type = other DNA
gtaagtatgcctttcactgcgaggggttctggagaggcttctgagctccccatggcccaggcaggcagcaggtctggggcaggaggggggttgtggag

SEQ ID NO: 70 - DNA, 123 bp, synthetic construct
  misc_feature 1..123: note = Synthetic polynucleotide
  source 1..123: mol_type = other DNA
tagcagctacaatccagctaccattctgcttttattttatggttgggataaggctggattattctgagtccaagctaggcccttttgctaatcatgttcatacctcttatcttcctcccacag

SEQ ID NO: 71 - DNA, 53 bp, synthetic construct
  misc_feature 1..53: note = Synthetic polynucleotide
  source 1..53: mol_type = other DNA
ctcctgggcaacgtgctggtctgtgtgctggcccatcactttggcaaagaatt

SEQ ID NO: 72 - DNA, 131 bp, synthetic construct
  misc_feature 1..131: note = Synthetic polynucleotide
  source 1..131: mol_type = other DNA
catcatgtgccttgactcggggcctggcccccccatctctgtcttgcaggacaattgccgtcttctgtctcgtggggcatcctcctgctggcaggcctgtgctgcctggtccctgtctccctggctgagga